(19) United States
(12) Patent Application Publication
(10) Pub. No.: US 2017/0143673 A1
(43) Pub. Date: May 25, 2017

(54) Methods For Treating Heart Failure Using Glucagon Receptor Antagonists

(71) Applicant: REMD Biotherapeutics, Inc., Camarillo, CA (US)

(72) Inventor: Hai Yan, Thousand Oaks, CA (US)

(21) Appl. No.: 15/255,707

(22) Filed: Sep. 2, 2016

Related U.S. Application Data
(60) Provisional application No. 62/259,066, filed on Nov. 24, 2015.

(51) Int. Cl. A61K 31/415; A61K 45/06

(52) U.S. Cl. CPC A61K 31/415; A61K 45/06

(57) ABSTRACT

The present invention relates to methods for treating or preventing heart failure using a glucagon receptor antagonist or modulator. In various embodiments, the present invention relates to methods for treating post-myocardial infarction heart failure and lateral ventricular (LV) remodeling. In various embodiments, the present invention relates to methods for treating diabetic cardiomyopathy.

### CROSS-REFERENCE TO RELATED APPLICATON

This application claims priority to provisional application U.S. Ser. No. 62/259,066, filed Nov. 24, 2015, hereby incorporated by reference.

### BACKGROUND

Heart failure (HF) is a global problem with an estimated prevalence of 38 million patients worldwide, including 6 million in the United States and more than 550,000 new patients diagnosed with the condition in the US each year. HF is the most common diagnosis in patients aged 65 years or older admitted to hospital and it is one of the most significant causes of morbidity and mortality in developed countries.

Heart failure is a clinical syndrome characterized by the failure of the heart to pump sufficient blood to meet the body's systemic demands. The heart contracts and relaxes with each heartbeat—these phases are referred to as systole (the contraction phase) and diastole (the relaxation phase). Systolic heart failure (SHF) is characterized by low ejection fraction. In patients with diastolic heart failure (DHF), contraction may be normal but relaxation of the heart may be impaired. This impairment is generally caused by a stiffening of the ventricles. Such impairment is referred to as diastolic dysfunction and if severe enough to cause pulmonary congestion (increased pressure and fluid in the blood vessels of the lungs), diastolic heart failure. DHF patients differ from those patients with SHF, in that DHF patients may have a “normal” ejection fraction. However, because the ventricle doesn't relax normally, the pressure within the ventricle increases and the blood filling the ventricle exceeds what is “normal”. People with certain types of cardiomyopathy may also have diastolic dysfunction.

Left ventricular hypertrophy (LVH) refers to a thickening of the left ventricle as a result of increased left ventricular load. LVH can be a significant marker for cardiovascular disorders and most common complications include arrhythmias, heart failure, ischemic heart disease, and sudden death. Although LVH increases naturally with age, it is more common in people who have high blood pressure or have other heart problems. Because LVH usually develops in response to hypertension, current treatment and prevention mainly includes managing hypertension. Typical diagnosis involves the use of echocardiograms (ECHO) and electrocardiograms (ECG).

Myocardial infarction (MI) is a leading cause for heart failure. The mechanism of an MI often involves the rupture of an atherosclerotic plaque leading to complete blockage of a coronary artery which resulted in the death or damage of heart muscle cells because the heart muscle cells do not receive enough oxygen. Diabetes mellitus (type 1 or 2), high blood pressure, dyslipidemia/high levels of blood cholesterol, particularly high low-density lipoprotein, low high-density lipoprotein, high triglycerides, and obesity have all been linked to myocardial infarction (Jay N. Cohn, et al., Journal of the American College of Cardiology, 35(3), 569-82, 2000). Long-term outcome after MI can be largely be defined in terms of its impact on the size and shape of the left ventricle (i.e., LV remodeling). Three major mechanisms contribute to LV remodeling: i) early infarct expansion, ii) subsequent infarct extension into adjacent non infarcted myocardium, and iii) late hypertrophy in the remote LV (Id). As the heart remodels, it not only gets bigger, but the cardiac walls get thinner and the pumping capacity of the heart declines. Cardiac remodeling is generally accepted as a determinant of survival after recovery from MI. Although the importance of remodeling as a pathogenic mechanism is incompletely understood, cardiac remodeling is thought to be an important aspect of disease progression in HF, regardless of cause (Id). Left ventricular remodeling is the process by which ventricular size, shape, and function are regulated by mechanical, neuro-hormonal, and genetic factors. Remodeling may be physiological and adaptive during normal growth or pathological due to MI, cardiomyopathy, hypertension, or valvular heart disease (French and Kramer, Drug Discov. Today Dis Mech., 4(3): 185-196, 2007).

It was recently reported that glucagon receptor signaling in cardiomyocytes modulates outcomes in non-diabetic mice with experimental myocardial infarction (Ali, et al., Molecular Metabolism, 4:132-143, 2015). Specifically, exogenous glucagon administration directly impaired recovery of ventricular pressure in ischemic mouse hearts ex vivo, and increased mortality from myocardial infarction after LAD coronary artery ligation in mice in a p38 MAPK-dependent manner In contrast, cardiomyocyte specific reduction of glucagon action in adult GCGR^{CM-/-} mice (mice having GCGR inactivated) significantly improved survival, and reduced hypertrophy and infarct size following myocardial infarction (Id.) The cardiovascular consequences of manipulating glucagon action remain poorly understood.

Current drug treatments available for management of heart failure, and other cardiovascular disorders, include vasodilators to reduce the blood pressure and ease the workload of the heart, diuretics to reduce fluid overload, inhibitors and blocking agents of the body's neuro-hormonal responses (e.g., angiotensin-converting enzyme (ACE) inhibitors and beta-adrenergic blocking agents), and other medicaments. Such medications, while effective for a short time, often cannot be used for extended periods because of side effects. Various surgical procedures such as heart transplantation have also been proposed for patients who suffer from severe, refractory heart failure. Alternatively, an implantable medical device such as ventricular assist devices (VADs) may be implanted in the chest to increase the pumping action of the heart, or an intra-aortic balloon pump (IABP) may be used for maintaining heart function for short periods of time, but typically no longer than one month. While each of these approaches have proven to be at least partly beneficial to patients, they each have shortcomings which limit their overall effectiveness. For example, drug therapies often involve unwanted side effects and complex therapy regimens which contribute to poor patient compliance. And both drug therapy and surgical approaches are very costly, adding to the health care costs associated with heart failure. Despite the ongoing research and development of treatments for heart failure, there is still a tremendous need for improved and alternative treatments.

### SUMMARY OF THE INVENTION

The present invention is based on the inventors' unique insight that glucagon receptor antagonists or modulators provide for improved, effective therapies for treatment and prevention of heart failure after myocardial infarction. The beneficial therapeutic effects provided by blocking the glucagon receptor in myocardial infarction-induced heart failure subjects (or all other heart failure subjects) relate to the prevention or attenuation of left ventricular (LV) remodeling which may include: increasing fractional shortening (FS), decreasing LV dilation (LVESD); preserving LV wall thickness; increasing LV developed pressure and ventricular contractility; decreasing the ratio of heart weight to body weight (infarct size); reducing the fibrosis process; reducing levels of incompletely oxidized fatty acid metabolites in the heart under ischemic conditions; and reducing MI-induced mortality rate.

Thus, in one aspect, the present invention comprises a method for treating or preventing heart failure and associated conditions in a subject, comprising administering to a subject diagnosed with heart failure, or a subject at risk of contracting heart failure, a therapeutically effective amount of a glucagon receptor antagonist or modulator.

In further embodiments, the present invention comprises a method for treating or preventing heart failure after myocardial infarction. In further embodiments, the present invention comprises a method for treating or preventing heart failure associated with diabetes mellitus. In further embodiments, the present invention comprises a method for treating or preventing heart failure associated with diabetic cardiomyopathy. In further embodiments, the present invention comprises a method of treating lateral ventricular (LV) remodeling.

A further embodiment of the invention comprises the use of glucagon receptor antagonists or modulators, as described in U.S. Pat. Nos. 8,907,103, 8,445,538, 8,361,959, 9,045,389, 8,623,818, 7,138,529, 8,748,624, 8,232,413, 8,470,773, 8,324,384, 8,809,579, 8,318,667, 8,735,604, 789472, 7,935,713, 7,803,951, 7,687,534, and 8,436,015, U.S. Patent Application Nos. 20140135400, 20110281795, 20130012493, and 20130012434, and PCT Application Nos. WO2010019828, WO2003051357, WO2015066252, WO2003053938, WO2004/069158, WO2005/121097, and WO2007/015999.

A further embodiment of the invention comprises the use of a glucagon receptor modulator as described in U.S. Pat. Nos. 8,084,489, 7,816,557, 7,807,702, 8,691,856, 7,863,329, 8,076,374, 7,696,248, 7,989,457, 8,809,342, 8,507,533 and 8,927,577.

A further embodiment of the invention comprises the use of a glucagon receptor antagonist selected from LY2409021, MK-0893, GRA1, LGD-6972, PF-06291874 and Bat 27-9955.

In various embodiments, the present invention comprises a method for treating or preventing heart failure and associated conditions in a subject, comprising administering to a subject diagnosed with heart failure, or a subject at risk of developing heart failure, a therapeutically effective amount of a glucagon receptor antagonist or modulator; and (b) a second agent composition. In various embodiments, the second agent composition is selected from a group consisting of: angiotensin-converting enzyme (ACE) inhibitors, β-adrenergic blocking agents, angiotension II receptor blockers (ARBs), diuretics, and digitalis.

### MODE(S) FOR CARRYING OUT THE INVENTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those commonly used and well known in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those commonly used and well known in the art. Standard techniques are used for chemical syntheses, chemical analyses, pharmaceutical preparation, formulation, and delivery, and treatment of subjects.

### Cardiovascular Diseases/Diabetic Cardiomyopathy/Heart Failure

Cardiovascular disease is a general name for a wide variety of diseases, disorders and conditions that affect the heart and/or blood vessels. Types of cardiovascular disease includes angina, heart attack (myocardial infarction), atherosclerosis, heart failure, cardiovascular disease, rheumatic heart disease, cardiac arrhythmias (abnormal heart rhythms), cerebrovascular disease, congenital heart defects, cardiomyopathy, left ventricular hypertrophy, right ventricular hypertrophy, post-infarction heart rupture, infections of the heart, coronary artery disease, peripheral arterial disease, renal artery stenosis, aortic aneurysm, myocardial diseases, heart valve disorders, myocarditis, and pericarditis.

In one aspect, the present invention comprises a method for treating or preventing cardiovascular disease induced heart failure in a subject, comprising administering to the subject a therapeutically effective amount of a glucagon receptor antagonist.

In various embodiments, the present invention comprises a method for treating or preventing heart failure and associated conditions in a subject, comprising administering to a subject diagnosed with heart failure, or a subject at risk of contracting heart failure, a therapeutically effective amount of a glucagon receptor antagonist.

In further embodiments, the present invention comprises a method of treating lateral ventricular (LV) remodeling in a subject, comprising administering to a subject diagnosed with heart failure, or a subject at risk of contracting heart failure, a therapeutically effective amount of a glucagon receptor antagonist.

In further embodiments, the present invention comprises a method of treating post-myocardial infarction heart failure in a subject in need thereof comprising administering to a subject with myocardial infarction a therapeutically effective amount of a glucagon receptor antagonist.

Diabetic cardiomyopathy describes diabetes-associated changes in the structure and function of the myocardium that is not directly attributable to other confounding factors such as coronary artery disease (CAD) or hypertension. It has been reported that in many patients with type 2 diabetes, diabetes associated changes are amplified by the existence of these comorbidities, which likely will augment the development of left ventricular hypertrophy, increase the susceptibility of the heart to ischemic injury and increase the overall likelihood of developing heart failure (Boudina and Abel, Rev Endocr Metab Disord., 11(1): 31-39, 2010 March). Several mechanisms have been implicated in the pathogenesis of diabetic cardiomyopathy, and the mechanisms responsible for diabetic cardiomyopathy continue to be elucidated. Changes in myocardial structure, calcium signaling and metabolism are early defects that have been described mainly in animal models and may precede clinically manifest cardiac dysfunction (Id). Diabetic cardiomyopathy in humans is characterized by diastolic dysfunction (DD), which may precede the development of systolic dysfunction (SD).

Diabetic nephropathies are nerve damaging disorders that are a common serious complication of diabetes mellitus. There are four main types of diabetic neuropathy: peripheral, autonomic, radiculoplexus, and mononeuropathy. Individuals may have just one type or symptoms of several types. Most symptoms develop gradually, and problems may not be noticed until considerable damage has occurred. Prolonged exposure to high blood sugar can damage delicate nerve fibers, causing diabetic neuropathy. Accordingly, it may to possible to prevent diabetic neuropathy or slow its progress with tight blood sugar control.

In various embodiments, the present invention comprises a method for treating or preventing heart failure and associated conditions in a subject, comprising administering to a subject diagnosed with diabetes mellitus or a subject at risk of developing diabetes mellitus, a therapeutically effective amount of a glucagon receptor antagonist.

In various embodiments, the present invention comprises a method for treating or preventing heart failure and associated conditions in a subject, comprising administering to a subject diagnosed with diabetic cardiomyopathy or a subject at risk of developing diabetic cardiomyopathy, a therapeutically effective amount of a glucagon receptor antagonist.

### Definitions

As used herein, “heart failure” means an abnormality of cardiac function where the heart does not pump blood at the rate needed for the requirements of metabolizing tissues. Heart failure includes a wide range of disease states such as congestive heart failure, myocardial infarction, tachyarrhythmia, familial hypertrophic cardiomyopathy, ischemic heart disease, idiopathic dilated cardiomyopathy, myocarditis and the like. The heart failure can be caused by any number of factors, including, without limitation, ischemic, congenital, rheumatic, viral, toxic or idiopathic forms. Chronic cardiac hypertrophy is a significantly diseased state which is a precursor to congestive heart failure and cardiac arrest.

The terms “glucagon receptor antagonist” and “glucagon receptor modulator” are defined herein as a compound able to bind to the glucagon receptor and thereby antagonize the activity of glucagon mediated by the glucagon receptor. Inhibiting the activity of glucagon by antagonizing the binding and activity of glucagon at the glucagon receptor reduces the rate of gluconeogenesis and glycogenolysis, and the concentration of glucose in plasma. Methods by which to determine the binding of a supposed antagonist with the glucagon receptor are known in the art and means by which to determine the interference with glucagon activity at the glucagon receptor are publicly available; see, e.g., S. E. de Laszlo et ah, 1999 Bioorg. Med. Chem. Lett. 9:641-646. The present invention is particularly directed to glucagon receptor antagonists having as a functional component thereof a small molecule compound, or in other words a low molecular weight organic compound. A small molecule is typically less than 800 Daltons.

The glucagon receptor antagonist in specific individual embodiments is selected from one of the following patent publication disclosures: U.S. Pat. Nos. 8,907,103, 8,445,538, 8,361,959, 9,045,389, 8,623,818, 7,138,529, 8,748,624, 8,232,413, 8,470,773, 8,324,384, 8,809,579, 8,318,667, 8,735,604, 789,472, 7,935,713, 7,803,951, 7,687,534, and 8,436,015, U.S. Patent Application Nos. 20140135400, 20110281795, 20130012493, and 20130012434, and PCT Application Nos. WO2010019828, WO2003051357, WO2015066252, WO2003053938, WO2004/069158, WO2005/121097, and WO2007/015999, although by no means limited hereto.

A further embodiment of the invention is a glucagon receptor modulator as disclosed in U.S. Pat. Nos. 8,084,489, 7,816,557, 7,807,702, 8,691,856, 7,863,329, 8,076,374, 7,696,248, 7,989,457, 8,809,342, 8,507,533 and 8,927,577, although by no means limited hereto.

The term “blood glucose level”, or “level of blood glucose” shall mean blood glucose concentration. In certain embodiments, a blood glucose level is a plasma glucose level. Plasma glucose may be determined in accordance with, e.g., Etgen et al., Metabolism, 49(5): 684-688, 2000) or calculated from a conversion of whole blood glucose concentration in accordance with D'Orazio et al., Clin. Chem. Lab. Med., 44(12):1486-1490, 2006.

A “pharmaceutical composition” refers to a composition suitable for pharmaceutical use in an animal or human. A pharmaceutical composition comprises a pharmacologically and/or therapeutically effective amount of an active agent and a pharmaceutically acceptable carrier. “Pharmaceutically acceptable carrier” refers to compositions that do not produce adverse, allergic, or other untoward reactions when administered to an animal or a human As used herein “pharmaceutically acceptable carrier” refers to any of the standard pharmaceutical carriers, vehicles, buffers, and carriers, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

As used herein, a “therapeutically effective amount” of a glucagon receptor antagonist refers to an amount of such compound that, when provided to a subject in accordance with the disclosed and claimed methods effects one of the following biological activities: treats heart failure; or reduces, suppresses, attenuates, or inhibits one or more symptoms of heart failure.

The terms “treat”, “treating” and “treatment” refer refers to an approach for obtaining beneficial or desired clinical results. Further, references herein to “treatment” include references to curative, palliative and prophylactic treatment.

As used herein and in the appended claims, the singular forms “a,” “or,” and “the” include plural referents unless the context clearly dictates otherwise. It is understood that aspects and variations of the invention described herein include “consisting” and/or “consisting essentially of” aspects and variation.

Reference to “about” a value or parameter herein includes (and describes) variations that are directed to that value or parameter per se. For example, description referring to “about X” includes description of “X”.

A further embodiment of the present invention is a glucagon receptor antagonist selected from the group consisting of a compound selected from LY2409021, MK-0893, GRA1, LGD-6972, PF-06291874, Bat 27-9955; [N-[(4-{(1S)-1-[3-(3, 5-dichlorophenyl)-5-(6-methoxynaphthalen-2-yl)-1H-pyrazol-1-yl]ethyl}phenyl) carbonyl]-β-alanine]; [N-(4-{(1S)-1-[(R)-(4-chlorophenyl)(7-fluoro-5-methyl-1H-indo1-3-yl)methyl]butyl}benzoyl)-β-alanine];

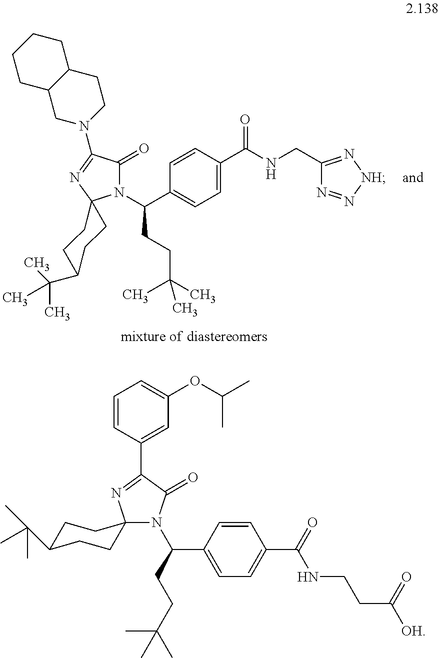 [SMILES: CC(C)(C)CC[C@H](c1ccc(C(=O)NCc2nn[nH]n2)cc1)N1C(=O)C(N2CCC3CCCCC3C2)=NC12CCC(C(C)(C)C)CC2 ; CC(C)Oc1cccc(C2=NC3(CCC(C(C)(C)C)CC3)N([C@H](CCC(C)(C)C)c3ccc(C(=O)NCCC(=O)O)cc3)C2=O)c1]

A further embodiment of the present invention is a glucagon receptor antagonist selected from the group consisting of:
2-{4-[2-(4-tert-butyl-phenyl)-2-(2′,4′-dichloro-biphenyl-4-yl-carbamoyl)-ethyl]-benzoylamino}-ethanesulfonate, sodium salt;
2-{4-[2-(4-benzofuran-2-yl-phenylcarbamoyl)-2-(4-tert-butyl-phenyl)-ethyl]-benzoylamino}-ethanesulfonate, sodium salt;
4-[2-(4-Benzofuran-2-yl-phenylcarbamoyl)-2-(4-cyclohex-1-enyl-phenyl)-ethyl]-benzoylamino-ethane sulfonic acid sodium salt;
4-[2-(4-Benzofuran-2-yl-phenylcarbamoyl)-2-(4-cyclohexyl-phenyl)-ethyl]-benzoylamino-ethane sulfonic acid sodium salt;
2-{4-[2-(4-tert-Butylphenyl)-2-(4-cyclohexylmethoxyphenylcarbamoyl)-ethyl]-phenoxy}-ethanesulfonic acid;
2-{4-[2-(4-tert-Butylphenyl)-2-(4-cyclohexylmethoxyphenylcarbamoyl)-propyl]-phenoxy}-ethanesulfonic acid;
2-{4-[(4-tert-Butyl-phenyl)-(4-cyclohexylmethoxy-phenylcarbamoyl)-methylsulfanyl]-benzoylamino}-ethanesulfonic acid;
2-{4-[(4-tert-Butyl-phenyl)-(4-cyclohexylmethoxy-phenylcarbamoyl)-methanesulfonyl]-benzoylamino}-ethanesulfonic acid;
2-{4-[(4-tert-Butyl-phenyl)-(4-cyclohexylmethoxy-phenylcarbamoyl)-methanesulfinyl]-benzoylamino}-ethanesulfonic acid;
2-{4-[(4-Benzofuran-2-yl-phenylcarbamoyl)-(4-tert-butylphenyl)-methoxy]-benzoylamino}-ethanesulfonic acid;
2-{4-[(4-Benzofuran-2-yl-phenylcarbamoyl)-(4-tert-butyl-phenyl)-methylsulfanyl]-benzoylamino}-ethanesulfonic acid;
2-{4-[(4-Benzofuran-2-yl-phenylcarbamoyl)-(4-tert-butyl-phenyl)-methoxy]-3-fluoro-benzoylamino}-ethanesulfonic acid;
2-({5-[2-(4-Benzofuran-2-yl-phenylcarbamoyl)-2-(4-tert-butyl-phenyl)-ethyl]-thiophene-2-carbonyl}-amino)-ethanesulfonic acid;
2-({5-[2-(4-tert-Butyl-phenyl)-2-(2′,4′-dichloro-biphenyl-4-ylcarbamoyl)-ethyl]-thiophene-2-carbonyl}-amino)-ethanesulfonic acid;
2-{4-[(E)-2-(4-tert-Butylphenyl)-2-(4-cyclohexylmethoxy-phenylcarbamoyl)-vinyl]-benzoylamino}-ethanesulfonic acid;
2-{4-[(Z)-2-(4-tert-Butylphenyl)-2-(4-cyclohexylmethoxy-phenylcarbamoyl)-vinyl]-benzoylamino}-ethanesulfonic acid;
2-(4-{[(4-Benzofuran-2-yl-phenylcarbamoyl)-(4-tert-butyl-phenyl)-methyl]-methylamino}-benzoylamino)-ethanesulfonic acid;
2-(4-{[Cyclohex-1-enyl-(4′-trifluoromethyl-biphenyl-4-ylcarbamoyl)-methyl]-amino}-benzoylamino)-ethanesulfonic acid;
N-{4-[2-(4-Benzofuran-2-yl-phenylcarbamoyl)-2-(4-tert-butyl-phenyl)-ethyl]-phenyl}-succinamic acid;
{4-[2-(4-Benzofuran-2-yl-phenylcarbamoyl)-2-(4-tert-butyl-phenyl)-ethyl]-phenylcarbamoyl}-methanesulfonate sodium salt;
2-{4-[2-(4-Benzofuran-2-yl-phenyl carbamoyl)-2-(4-tert-butyl-phenyl)-ethyl]-phenylcarbamoyl}-ethanesulfonate sodium salt;
2-{4-[2-(4-Benzofuran-2-ylphenylcarbamoyl)-3-(cyclohex-2-enylphenyl)-propyl]-benzoylamino}-ethanesulfonic acid;
2-{4-[2-(4-Benzofuran-2-yl-2-fluorophenylcarbamoyl)-hept-4-ynyl]-benzoylamino}-ethanesulfonic acid;
(R)-2-(4-{2-(4-Benzofuran-2-yl-phenylcarbamoyl)-2-[443,3 -dimethyl-but-1-enyl)-phenyl]-ethyl}-benzoyl amino-ethanesulfonic acid sodium salt;
(S)-2-(4-{2-(4-Benzofuran-2-yl-phenylcarbamoyl)-2-[4-(3,3-dimethyl-but-1-enyl)-phenyl]-ethyl}-benzoyl amino-ethane sulfonic acid sodium salt;
2-{4-[2-[5-(4-Bromo-phenyl)-isoxazol-3-yl]-2-(4-tert-butyl-phenyl)-ethyl]-benzoylamino}-ethanesulfonic acid;
2-(4-{2-(4-tert-Butyl-phenyl)-2-[5-(2′,4′-dichlorobiphenyl-4-yl)-isoxazol-3-yl]-ethyl}-benzoylamino)-ethanesulfonic acid;
(S)-2-(4-{2-(4-tert-Butyl-phenyl)-2-[5-(4′-chloro-biphenyl-3-yl)-isoxazol-3-yl]-ethyl}-benzoylamino)-ethanesulfonic acid;
(R)-2-(4-{2-(4-tert-Butyl-phenyl)-2-[5-(4′-chloro-biphenyl-3-yl)-isoxazol-3-yl-ethyl}-benzoylamino)-ethanesulfonic acid;
Sodium; 2-{4-[2-[4-(4-tert-butyl-cyclohex-1-enyl)-phenyl]-2-(4′-chloro-2′-methyl-biphenyl-ylcarbamoyl)-ethyl]-benzoylamino}-ethanesulfonate;
Sodium-2-(4-{2-(4′-chloro-2′-methyl biphenyl-4-ylcarbamoyl)-2-[3-(4,4-dimethyl-cyclohexyl)-phenyl]-ethyl}-benzoylamino-ethanesulfonic acid;
Ammonium, 2-(S)-{4-[2-[4-(4-(R)-tert-butylcyclohex-1-enyl)-phenyl]-2-(4′-chloro-2′-methyl biphenyl-4-ylcarbamoyl)-ethyl]-benzoylamino}-ethanesulfonate;
Ammonium, 2-(R)-{4-[2-[4-(4-(R)-tert-butylcyclohex-1-enyl)-phenyl]-2-(4′-chloro-2′-methylbiphenyl-4-ylcarbamoyl)-ethyl]-benzoylamino}-ethanesulfonate;
Ammonium, 2-(R)-{4-[2-[4-(4-(S)-tert-butylcyclohex-1-enyl)-phenyl]-2-(4′-chloro-2′-methylbiphenyl-4-ylcarbamoyl)-ethyl]-benzoylamino}-ethanesulfonate;
Sodium-2-(R)-{4-[2-(4′-chloro-2′-methyl-biphenyl-4-ylcarbamoyl)-2-[(4-(3,3-dimethyl-but-1-enyl)-phenyl]-ethyl}-benzoylamino ethanesulfonic acid;
Sodium-2-(S)-4-[2-(4′-chloro-2′-methyl-biphenyl-4-ylcarbamoyl)-2-(4-(3,3-dimethyl-but-1-enyl)-phenyl]-ethyl}-benzoylamino ethanesulfonic acid;
2-(4-{(R)-2-(4′-Chloro-2′-methyl-biphenyl-4-ylcarbamoyl)-2-[4-(4,4-dimethyl-cyclohex-1-enyl)-phenyl]-ethyl}-benzoylamino)-thanesulfonic acid;
2-(4-{(S)-2-(4′-Chloro-2′-methyl-biphenyl-4-ylcarbamoyl)-24-[4-(4,4-dimethyl-cyclohex-1-enyl)-phenyl]-ethyl}-benzoylamino)-ethanesulfonic acid;
Sodium-2-[4-(S)-2-(4′-tert-butyl-biphenyl-4-yl)-2-(4′-chloro-2′-methyl-phenyl-carbamoyl)-ethyl}-benzoylamino-ethanesulfonic acid;
Sodium-2-[4-(R)-2-(4′-tert-butyl-biphenyl-4-yl)-2-(4′-chloro-2′-methyl-phenyl-carbamoyl)-ethyl}-benzoylamino-ethanesulfonic acid;
Sodium-2-[4-(R)-2-(4′-tert-butyl-biphenyl-4-yl)-2-(2″,4′,6′-trimethyl-biphenyl-4-ylcarbamoyl)-ethyl}-benzoylamino-ethanesulfonic acid;
Ammonium-2-(R)-(4-{2-[4′-tert-butyl-biphenyl-4-yl)-2-[3-(4,4-dimethyl-cyclohex-1-enyl)-phenyl-4-ylcarbamoyl]-ethyl}-benzoylamino-ethanesulfonic acid;
2-(4-[2-(4-Benzooxazol-2-yl-phenylcarbamoyl)-2-4-4-(1R,4R)-1,7,7-trimethyl-bicyclo [2,2,1]hept-2-en-2-yl)-phenyl]ethyl-benzoylamino)-ethanesulfonic acid;
Sodium-2-[4-(R)-2-(4′-tert-butyl-biphenyl-4-yl)-2-(4′-chloro-3′-methyl-phenyl-carbamoyl)-ethyl}-benzoylamino-ethanesulfonic acid;
Ammonium-2-(R)-(4-{2-[4′-tert-butyl-biphenyl-4-yl)-2-(4-methyl-benzooxazol-2-yl)phenylcarbamoyl]-ethyl}-benzoylamino)-ethanesulfonic acid;
Ammonium-2-(S)-(4-{2-[4′-tert-butyl-biphenyl-4-yl)-2-(4-methyl-benzooxazol-2-yl)phenylcarbamoyl]-ethyl}-benzoylamino)-ethanesulfonate;
2-{4-[(R)-24-[-(4-(cis)-tert-Butylcyclohexyl)-phenyl]-2-(4′-chloro-2′-methyl-biphenyl-4-ylcarbamoyl)-ethyl]-benzoylamino}-ethanesulfonic acid;
N-(4-{(1S)-1-[(4-chlorophenyl)(4-fluoro-1-naphthyl)methyl]butyl}benzoyl)-(3-alanine;
N-(4-{(1S)-1-[(4-chlorophenyl)(6-methoxy-2-naphthyl)methyl]butyl}benzoyl)-(3-alanine;
N-[4-((1S)-1-(4-chlorophenyl)[6-(difluoromethoxy)-2-naphthyl]methyl}butyl)benzoyl]-β-alanine;
N-(4-{(1S)-1-[(4-chlorophenyl)(6-cyano-2-naphthyl)methyl]butyl}benzoyl)-(3-alanine;
N-(4-{(1S)-1-[(4-cyanophenyl)(4-fluoro-1-naphthyl)methyl]butyl}benzoyl)-(3-alanine;
N-(4-{(1S)-1-[(4-chlorophenyl)(5-cyano-7-methyl-1-naphthyl)methyl]butyl}benzoyl)-(3-alanine;
(2S)-3-[(4-{(1S)-1-[(4-chlorophenyl)(6-methoxy-2-naphthyl)methyl]butyl}benzoyl)amino]-2-hydroxypropanoic acid;
N-(4-{(1S)-1-[(4-fluoro-1-naphthyl)(4-methoxyphenyl)methyl]butyl}benzoyl)-(3-alanine;
N-(4-{(1S)-1-[(4-chlorophenyl)(1-naphthyl)methyl]butyl}benzoyl)-(3-alanine;
N-(4-{(1S)-1-[(4-bromo-1 -naphthyl)(4-chlorophenyl)methyl]butyl}benzoyl)-(3-alanine;

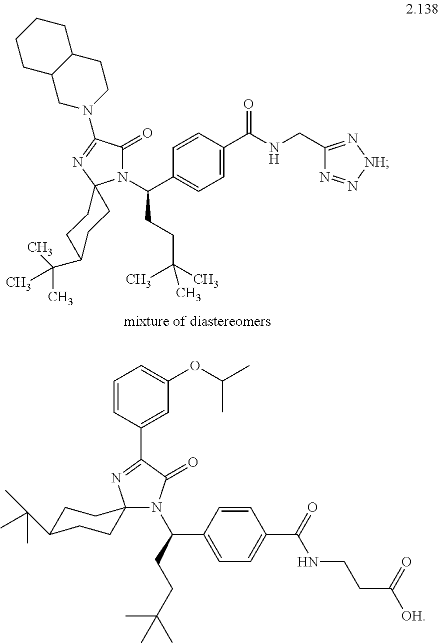 [SMILES: CC(C)(C)CC[C@H](c1ccc(C(=O)NCc2nn[nH]n2)cc1)N1C(=O)C(N2CCC3CCCCC3C2)=NC12CCC(C(C)(C)C)CC2 ; CC(C)Oc1cccc(C2=NC3(CCC(C(C)(C)C)CC3)N([C@H](CCC(C)(C)C)c3ccc(C(=O)NCCC(=O)O)cc3)C2=O)c1]

3-(2′-(((2,4′-dichloro-2′-methyl-[1,1′-biphenyl]-4-yl)amino)methyl)-5′-(trifluoromethyl)-[1,1′-biphenyl]-4-ylcarboxamido)propanoic acid;
3-(2′-(((2-chloro-2′-methyl-4′-(trifluoromethyl)-[1,1′-biphenyl]-4-yl)amino)methyl)-5′-(trifluoromethyl)-[1,1′-biphenyl]-4-ylcarboxamido)propanoic acid;
3-(2′-(((2,2′-dichloro-4′-(trifluoromethyl)-[1,1′-biphenyl]-4-yl)amino)methyl)-5′-(trifluoromethyl)-[1,1′-biphenyl]-4-ylcarboxamido)propanoic acid;
3-(2′-(((2′,4′-dichloro-[1,1′-biphenyl]-4-yl)amino)methyl)-5′-(trifluoromethyl)-[1,1′-biphenyl]-4-ylcarboxamido)propanoic acid;
3-(2′-(((2,4′-dichloro-2′-methyl-[1,1′-biphenyl]-4-yl)amino)methyl)-2-methyl-5′-(trifluoromethyl)-[1,1′-biphenyl]-4-ylcarboxamido)propanoic acid;
3-(2′-(((2-chloro-2′-methyl-4′-(trifluoromethyl)-[1,1′-biphenyl]-4-yl)amino)methyl)-2-methyl-5′-(trifluoromethyl)-[1,1′-biphenyl]-4-ylcarboxamido)propanoic acid;
3-(2′-(((4′-chloro-[1,1′-biphenyl]-4-yl)amino)methyl)-2-methyl-[1,1′-biphenyl]-4-ylcarboxamido)propanoic acid;
3-(5′-chloro-2′-(((2′,4′-dichloro-[1,1′-biphenyl]-4-yl)amino)methyl)-[1,1′-biphenyl]-4-ylcarboxamido)propanoic acid;
3-(2′-(((4′-fluoro-3′-nitro-[1,1′-biphenyl]-4-yl)amino)methyl)-[1,1′-biphenyl]-4-ylcarboxamido)propanoic acid;
3-(5′-chloro-2′-(((4′-chloro-[1,1′-biphenyl]-4-yl)amino)methyl)-3-fluoro-[1,1′-biphenyl]-4-ylcarboxamido)propanoic acid;
3-(5¹-chloro-3-fluoro-2′-(((4′-fluoro-[1,1′-biphenyl]-4-yl)amino)methyl)-[1,1′-biphenyl]-4-ylcarboxamido)propanoic acid;
3-(5′-chloro-2′-(((2′,4′-dichloro-[1,1′-biphenyl]-4-yl)amino)methyl)-3-fluoro-[1,1′-biphenyl]-4-ylcarboxamido)propanoic acid;
3-(5′-chloro-2′-(1-((4′-chloro-[1,1′-biphenyl]-4-yl)amino)ethyl)-[1,1′-biphenyl]-4-ylcarboxamido)propanoic acid;
3-(5′-chloro-2′-(1-((4′-fluoro-[1,1′-biphenyl]-4-yl)amino)ethyl)-[1,1′-biphenyl]-4-ylcarboxamido)propanoic acid;
3-(2′-(((2,4′-dichloro-[1,1′-biphenyl]-4-yl)amino)methyl)-3/-methoxy-5′-(trifluoromethyl)-[1,1′-biphenyl]-4-lcarboxamido)propanoic acid;
3-(2′-(((2′-chloro-4′-(trifluoromethyl)-[1,1′-biphenyl]-4-yl)amino)methyl)-3/-methoxy-5′-(trifluoromethyl)-[1,1′-biphenyl]-4-ylcarboxamido)propanoic acid;
3-(2′-(((2-chloro-2′-methyl-4′-(trifluoromethyl)-[1,1′-biphenyl]-4-yl)amino)methyl)-3′-isopropyl-5′-(trifluoromethyl)-[1,1′-biphenyl]-4-ylcarboxamido)propanoic acid;
3-(2′-(((4′-chloro-[1,1′-biphenyll-4-yl)amino)methyl)-[1,1′-biphenyl]-4-ylcarboxamido)propanoic acid;
3-(4-((1R,2S)-1-(5-CHLORO-7-FLUORO-1H-INDOL-3-YL)-1-(4-(TRIFLUOROMETHOXY)PHENYL)PENTAN-2-YL)BENZAMIDO)PROPANOIC ACID;
3-(4-((1S,2R)-1-(5-CHLORO-7-FLUORO-1H-INDOL-3-YL)-1-(4-(TRIFLUOROMETHOXY)PHENYL)PENTAN-2-YL)BENZAMIDO)PROPANOIC ACID;
3-(4-(1-(5-CHLORO-7-FLUORO-1H-INDOL-3-YL)-1-(4-(TRIFLUOROMETHOXY)PHENYL)PENTAN-2-YL)BENZAMIDO)PROPANOIC ACID;
N-(3 -Cyano-4,5 -dimethyl-thiophen-2-yl)-2,2-diphenyl-acetamide;
N-(3-Cyano-4,5-dimethyl-thiophen-2-yl)-2,3-diphenyl-propionamide;
N-(3 -Cyano-4-methyl-5-ethyl-thiophen-2-yl)-2,3-diphenyl-propionamide;
N-(3-Cyano-4,7-dihydro-5H-thieno[2,3-c]pyran-2-yl)-2,2-diphenyl-acetamide;
3-Cyano-2-diphenylacetylamino-4,5,6,7-tetrahydro-5H-thieno[2,3-c]pyridine-6-carboxylic acid tert-butyl ester;
3-Cyano-2-(2-phenyl-propionylamino)-4,5,6,7-tetrahydro-5H-thieno[2,3-c]pyridine-6-carboxylic acid tert-butyl ester;
3-Cyano-2-(3-methyl-2-phenyl-butyrylamino)-4,5,6,7-tetrahydro-5H-thieno[2,3-c]pyridine-6-carboxylic acid tert-butyl ester;
3-Cyano-2-(3-methyl-2-phenyl-pentanoylamino)-4,5,6,7-tetrahydro-5H-thieno[2,3-c]pyridine-6-carboxylic acid tert-butyl ester;
N-(3-Cyano-6-methanesulfonyl-4,5,6,7-tetrahydro-thieno[2,3-c]pyridin-2-yl)-2,2-diphenyl-acetamide;
N-(3-Cyano-6-methyl-4,5,6,7-tetrahydro-thieno[2,3-c]pyridin-2-yl)-2,2-diphenyl-acetamide;
3-Cyano-2-diphenylacetylamino-4,5,6,7-tetrahydro-5H-thieno[2,3-c]pyridine-6-carboxylic acid methyl ester;
N-(3-Cyano-4,7-dihydro-5H-thieno[2,3-c]pyran-2-yl)-2-phenyl-butyramide;
3-Methyl-2-phenyl-pentanoic acid (3-cyano-5,6-dihydro-4H-cyclopenta[b]thiophen-2-yl)-amide;
3-Methyl-2-(2-phenyl-propionylamino)-(3-cyano-5,6-dihydro-4H-cyclopenta[b ]thiophen-2-yl)-amide;
N-(3-Cyano-5,6-dihydro-4H-cyclopenta[b]thiophen-2-yl)-2,2-diphenyl-acetamide;
N-(3-Cyano-5,6-dihydro-4H-cyclopenta[b]thiophen-2-yl)-2-cyclopentyl-2-phenyl-acetamide;
N-(3-Cyano-5,6-dihydro-4H-cyclopenta[b]thiophen-2-yl)-2,2-diphenyl-acetamide;
N-(3-Cyano-5,6-dihydro-4H-cyclopenta[b]thiophen-2-yl)-2-phenyl-propionamide;
3-(5-(2-(([1,1′-biphenyl]-4-ylamino)methyl)phenyl)picolinamido)propanoic acid;
3-(5-(2-(((4′-chloro-[1,1′-biphenyl]-4-yl)amino)methyl)phenyl)picolinamido)propanoic acid;
3-(5-(2-(((4′-(tert-butyl)-[1,1′-biphenyl]-4-yl)amino)methyl)phenyl)picolinamido)propanoic acid;
3-(5-(4-chloro-2-(((4′-chloro-[1,1′-biphenyl]-4-yl)amino)methyl)phenyl)picolinamido)propanoic acid;
3-(5-(4-chloro-2-(((2′,4′-dichloro-[1,1′-biphenyl]-4-yl)amino)methyl)phenyl)picolinamido)propanoic acid;
3-(5-(5-chloro-2-(((4′-chloro-[1,1′-biphenyl]-4-yl)amino)methyl)phenyl)picolinamido)propanoic acid;
3-(5-(5-chloro-2-(((2′,4′-dichloro-[1,1′-biphenyl]-4-yl)amino)methyl)phenyl)picolinamido)propanoic acid;
3-(5-(2-(((3′,4′-dichloro-[1,1′-biphenyl]-4-yl)amino)methyl)phenyl)picolinamido)propanoic acid;
3-(5-(2-(((4′-trifluoromethyl)-[1,1′-biphenyl]-4-yl)amino)methyl)phenyl)picolinamido)propanoic acid;
3-(5-(5-chloro-2-(((2,4′-dichloro-[1,1′-biphenyl]-4-yl)amino)methyl)phenyl)picolinamido)propanoic acid;
3-(5-(5-chloro-2-(((2′,4′-dichloro-3-fluoro-[1,1′-biphenyl]-4-yl)amino)methyl)phenyl)picolinamido) propanoic acid;
3-(5-(5-chloro-2-(((4′-chloro-3-fluoro-[1,1′-biphenyl]-4-yl)amino)methyl)phenyl)picolinamido) propanoic acid;
3-(5-(5-chloro-2-(((4′-chloro-3,3′-difluoro-[1,1′-biphenyl]-4-yl)amino)methyl)phenyl)picolinamido) propanoic acid;
3-(5-(5-chloro-2-(((3′-chloro-[1,1′-biphenyl]-4-yl)amino)methyl)phenyl)picolinamido)propanoic acid;
3-(5-(5-chloro-2-(((4′-fluoro-[1,1′-biphenyl]-4-yl)amino)methyl)phenyl)picolinamido)propanoic acid;
3-(5-(5-chloro-2-((4′-chloro-[1,1′-biphenyl]-4-yl)carbamoyl)phenyl)picolinamido)propanoic acid;
3-(5-(5-chloro-2-(((4′-chloro-[1,1′-biphenyl]-4-yl)(methyl)amino)methyl)phenyl)picolinamido) propanoic acid;
3-(5-(5-chloro-2-(((2′,4′-difluoro-[1,1′-biphenyl]-4-yl)amino)methyl)phenyl)picolinamido)propanoic acid;
3-(5-(5-chloro-2-(((2′,4′-dichloro-[1,1′-biphenyl]-4-yl)carbamoyl)phenyl)picolinamido)propanoic acid;
3-(5-(5-chloro-2-((4′-fluoro-[1,1′-biphenyl]-4-yl)carbamoyl)phenyl)picolinamido)propanoic acid;
3-(5-(2-(((2′,4′-dichloro-[1,1′-biphenyl]-4-yl)amino)methyl)-5-(trifluoromethyl)phenyl)picolinamido)propanoic acid;
3-(5-(2-(((4′-chloro-2′-methyl-[1,1′-biphenyl]-4-yl)amino)methyl)-5-(trifluoromethyl)phenyl)picolinamido)propanoic acid;
3-(5-(2-(((2′-chloro-4′-fluoro-[1,1′-biphenyl]-4-yl)amino)methyl)-5-(trifluoromethyl)phenyl)picolinamido)propanoic acid;
3-(5-(2-(((2′-chloro-4′-(trifluoromethyl)-[1,1′-biphenyl]-4-yl)amino)methyl)-5-(trifluoromethyl)phenyl)picolinamido)propanoic acid;
3-(5-(2-(((4′-chloro-2-(trifluoromethyl)-[1,1′-biphenyl]-4-yl)amino)methyl)-5-(trifluoromethyl)phenyl)picolinamido)propanoic acid;
3-(5-(2-(((4′-fluoro-3¹-(trifluoromethyl)-[1,1′-biphenyl]-4-yl)amino)methyl)-5-(trifluoromethyl)phenyl)picolinamido)propanoic acid;
3-(5-(2-(((2,2′,4′-trichloro-[1,1′-biphenyl]-4-yl)amino)methyl)-5-(trifluoromethyl)phenyl)picolinamido)propanoic acid;
3-(5-(2-(((2,4′-dichloro-[1,1′-biphenyl]-4-yl)amino)methyl)-5-(trifluoromethyl)phenyl)picolinamido)propanoic acid;
3-(5-(5-(trifluoromethyl)-2-(((4′-(trifluoromethyl)-[1,1′-biphenyl]-4-yl)amino)methyl)phenyl)picolinamido)propanoic acid;
3-(5-(2-(((T-methyl-4′-(trifluoromethyl)-[1,1′-biphenyl]-4-yl)amino)methyl)-5-(trifluoromethyl)phenyl)picolinamido)propanoic acid;
3-(5-(2-(((2-chloro-4′-fluoro-3′-(trifluoromethyl)-[1,1′-biphenyl]-4-yl)amino)methyl)-5-(trifluoromethyl)phenyl)picolinamido)propanoic acid;
3-(5-(2-(((2,4′-dichloro-3′-(trifluoromethyl)-[1,1′-biphenyl]-4-yl)amino)methyl)-5-(trifluoromethyl)phenyl)picolinamido)propanoic acid;
3-(5-(2-(((2,2′-dichloro-4′-fluoro-[1,1′-biphenyl]-4-yl)amino)methyl)-5-(trifluoromethyl)phenyl)picolinamido)propanoic acid;
3-(5-(2-(((2,4′-dichloro-2′-methyl-[1,1′-biphenyl]-4-yl)amino)methyl)-5-(trifluoromethyl)phenyl)picolinamido)propanoic acid;
3-(5-(2-(((2,3′-dichloro-4′-fluoro-[1,1′-biphenyl]-4-yl)amino)methyl)-5-(trifluoromethyl)phenyl)picolinamido)propanoic acid;
3-(5-(5-chloro-2-((2′,4′-difluoro-[1,1′-biphenyl]-4-yl)carbamoyl)phenyl)picolinamido)propanoic acid;
3-(5-(2-(((2,4′-dichloro-2′-fluoro-[1,1′-biphenyl]-4-yl)amino)methyl)-5-(trifluoromethyl)phenyl)picolinamido)propanoic acid;
3-(5-(2-(((2,2′-dichloro-3′-(trifluoromethyl)-[1,1′-biphenyl]-4-yl)amino)methyl)-5-(trifluoromethyl)phenyl)picolinamido)propanoic acid;
3-(5-(2-(((2-chloro-3′-fluoro-4′-(trifluoromethyl)-[1,1′-biphenyl]-4-yl)amino)methyl)-5-(trifluoromethyl)phenyl)picolinamido)propanoic acid;
3-(5-(2-(((4′-chloro-2′-fluoro-[1,1′-biphenyl]-4-yl)amino)methyl)-5-(trifluoromethyl)phenyl)picolinamido)propanoic acid;
3-(5-(2-(((2′-chloro-3′-(trifluoromethyl)-[1,1′-biphenyl]-4-yl)amino)methyl)-5-(trifluoromethyl)phenyl)picolinamido)propanoic acid;
3-(5-(2-(((2,2′-dichloro-4′-(trifluoromethyl)-[1,1′-biphenyl]-4-yl)amino)methyl)-5-(trifluoromethyl)phenyl)picolinamido)propanoic acid;
3-(5-(2-(((4′-chloro-[1,1′-biphenyl]-4-yl)amino)methyl)-5-(trifluoromethyl)phenyl)picolinamido)propanoic acid;
3-(5-(2-(((4′-chloro-[1,1′-biphenyl]-4-yl)amino)methyl)-5-methoxyphenyl)picolinamido)propanoic acid;
3-(5-(2-(((2′,4′-dichloro-[1,1′-biphenyl]-4-yl)amino)methyl)-5-methoxyphenyl)picolinamido)propanoic acid;
3-(5-(5-methoxy-2-(((4′-(trifluoromethyl)-[1,1′-biphenyl]-4-yl)amino)methyl)phenyl)picolinamido)propanoic acid;
3-(5-(5-methoxy-2-(((4′-(trifluoromethoxy)-[1,1′-biphenyl]-4-yl)amino)methyl)phenyl)picolinamido)propanoic acid;
3-(5-(2-(((4′-(tert-butyl)-[1,1′-biphenyl]-4-yl)amino)methyl)-5-methoxyphenyl)picolinamido)propanoic acid;
3-(5-(5-methoxy-2-(((3′-(trifluoromethoxy)-[1,1′-biphenyl]-4-yl)amino)methyl)phenyl)picolinamido)propanoic acid;
3-(5-(5-chloro-2-(((2,2′,4′-trichloro-[1,1′-biphenyl]-4-yl)amino)methyl)phenyl)picolinamido)propanoic acid;
3-(5-(5-chloro-2-(((2-chloro-4′-fluoro-[1,1′-biphenyl]-4-yl)amino)methyl)phenyl)picolinamido)propanoic acid;
3-(5-(2-(((3′,4′-dichloro-2-fluoro-[1,1′-biphenyl]-4-yl)amino)methyl)-5-fluorophenyl)picolinamido)propanoic acid;
3-(5-(5-chloro-2-(((2-methoxy-4′-(trifluoromethyl)-[1,1′-biphenyl]-4-yl)amino)methyl)phenyl)picolinamido)propanoic acid;
3-(5-(5-chloro-2-(((3′,4′-dichloro-2-(trifluoromethyl)-[1,1′-biphenyl]-4-yl)amino)methyl)phenyl)picolinamido)propanoic acid;
3-(5-(5-fluoro-2-(((2-fluoro-4′-(trifluoromethyl)-[1,1′-biphenyl]-4-yl)amino)methyl)phenyl)picolinamido)propanoic acid;
3-(5-(2-(((2′,4′-dichloro-2-fluoro-[1,1′-biphenyl]-4-yl)amino)methyl)-5-fluorophenyl)picolinamido)propanoic acid;
3-(5-(5-chloro-2-(((3′,4′-dichloro-[1,1′-biphenyl]-4-yl)amino)methyl)phenyl)picolinamido)propanoic acid;
3-(5-(2-(((4′-(tert-butyl)-[1,1′-biphenyl]-4-yl)amino)methyl)-5-chlorophenyl)picolinamido)propanoic acid;
3-(5-(2-(((2′,4′-dichloro-[1,1′-biphenyl]-4-yl)amino)methyl)-4-methoxyphenyl)picolinamido)propanoic acid;
3-(5-(2-(((3′,4′-dichloro-[1,1′-biphenyl]-4-yl)amino)methyl)-4-methoxyphenyl)picolinamido)propanoic acid;
3-(5-(2-(((3′-fluoro-[1,1′-biphenyl]-4-yl)amino)methyl)-5-(trifluoromethyl)phenyl)picolinamido)propanoic acid;
3-(5-(5-chloro-2-(((2-chloro-2′,4′-difluoro-[1,1′-biphenyl]-4-yl)amino)methyl)phenyl)picolinamido)propanoic acid;
3-(5-(2-(((4′-chloro-[1,1′-biphenyl]-4-yl)amino)methyl)-6-(trifluoromethyl)phenyl)picolinamido)propanoic acid;
3-(5-(2-(((4′-chloro-[1,1′-biphenyl]-4-yl)amino)methyl)-3-methylphenyl)picolinamido)propanoic acid;
3-(5-(2-(((4′-chloro-[1,1′-biphenyl]-4-yl)amino)methyl)-6-cyanophenyl)picolinamido)propanoic acid;
3-(5-(3-chloro-2-(((4′-chloro-[1,1′-biphenyl]-4-yl)amino)methyl)-5-(trifluoromethyl)phenyl)picolinamido)propanoic acid;
3-(5-(2-(((4′-chloro-[1,1′-biphenyl]-4-yl)amino)methyl)-5-methylphenyl)picolinamido)propanoic acid;
3-(5-(2-(((4′-chloro-[1,1′-biphenyl]-4-yl)amino)methyl)-6-methylphenyl)picolinamido)propanoic acid;
3-(5-(2-(((4′-chloro-[1,1′-biphenyl]-4-yl)amino)methyl)-5-(methylsulfonyl)phenyl)picolinamido)propanoic acid;
3-(5-(2-(((4′-chloro-[1,1′-biphenyl]-4-yl)amino)methyl)-3,5-bis(trifluoromethyl)phenyl)picolinamido)propanoic acid;
3-(5-(3-chloro-2-(((2′,4′-dichloro-[1,1′-biphenyl]-4-yl)amino)methyl)-5-(trifluoromethyl)phenyl)picolinamido)propanoic acid;
3-(5-(3-chloro-2-(((4′-chloro-2′-methyl-[1,1′-biphenyl]-4-yl)amino)methyl)-5-(trifluoromethyl)phenyl)picolinamido)propanoic acid;
3-(5-(3-chloro-2-(((2,4′-dichloro-[1,1′-biphenyl]-4-yl)amino)methyl)-5-(trifluoromethyl)phenyl)picolinamido)propanoic acid;
3-(5-(3-chloro-2-(((4′-chloro-2-methyl-[1,1′-biphenyl]-4-yl)amino)methyl)-5-(trifluoromethyl)phenyl)picolinamido)propanoic acid;
3-(5-(2-(((2,4′-dichloro-[1,1′-biphenyl]-4-yl)amino)methyl)-3,5-bis(trifluoromethyl)phenyl)picolinamido)propanoic acid;
3-(5-(4-chloro-2-(((4′-chloro-2′-methyl-[1,1′-biphenyl]-4-yl)amino)methyl)-5-(trifluoromethyl)phenyl)picolinamido)propanoic acid;
3-(5-(4-chloro-2-(((2,4′-dichloro-[1,1′-biphenyl]-4-yl)amino)methyl)-5-(trifluoromethyl)phenyl)picolinamido)propanoic acid;
3-(5-(4-chloro-2-(((4′-chloro-2-methyl-[1,1′-biphenyl]-4-yl)amino)methyl)-5-(trifluoromethyl)phenyl)picolinamido)propanoic acid;
3-(5-(4-chloro-2-(((4′-chloro-[1,1′-biphenyl]-4-yl)amino)methyl)-5-(trifluoromethyl)phenyl)picolinamido)propanoic acid;
3-(5-(2-(((4′-chloro-2′-methyl-[1,1′-biphenyl]-4-yl)amino)methyl)-3,5-bis(trifluoromethyl)phenyl)picolinamido)propanoic acid;
3-(5-(4-chloro-2-(((2′,4′-dichloro-[1,1′-biphenyl]-4-yl)amino)methyl)-5-(trifluoromethyl)phenyl)picolinamido)propanoic acid;
3-(5-(2-(((2′-methyl-4′-(trifluoromethyl)-[1,1′-biphenyl]-4-yl)amino)methyl)-3,5-bis(trifluoromethyl)phenyl)picolinamido)propanoic acid;
3-(5-(2-(((2′,3′-dichloro-[1,1′-biphenyl]-4-yl)amino)methyl)-3,5-bis(trifluoromethyl)phenyl)picolinamido)propanoic acid;
3-(5-(2-(((2′,6′-dichloro-[1,1′-biphenyl]-4-yl)amino)methyl)-3,5-bis(trifluoromethyl)phenyl)picolinamido)propanoic acid;
3-(5-(2-(((4′-chloro-2-methyl-[1,1′-biphenyl]-4-yl)amino)methyl)-3,5-bis(trifluoromethyl)phenyl)picolinamido)propanoic acid;
3-(5-(2-(((2′,4′-dichloro-[1,1′-biphenyl]-4-yl)amino)methyl)-3,5-bis(trifluoromethyl)phenyl)picolinamido)propanoic acid;
3-(5-(5-chloro-2-(((2,4′-difluoro-[1,1′-biphenyl]-4-yl)amino)methyl)phenyl)picolinamido)propanoic acid;
3-(5-(5-chloro-2-(((2,2′,4′-trifluoro-[1,1′-biphenyl]-4-yl)amino)methyl)phenyl)picolinamido)propanoic acid;
3-(5-(5-chloro-2-(((2-fluoro-4′-(trifluoromethyl)-[1,1′-biphenyl]-4-yl)amino)methyl)phenyl)picolinamido)propanoic acid;
3-(5-(5-chloro-2-(((4′-chloro-2-fluoro-[1,1′-biphenyl]-4-yl)amino)methyl)phenyl)picolinamido)propanoic acid;
3-(5-(5-chloro-2-(((2′,4′-dichloro-2-fluoro-[1,1′-biphenyl]-4-yl)amino)methyl)phenyl)picolinamido)propanoic acid;
3-(5-(5-chloro-2-(((4′-chloro-2,3′-difluoro-[1,1′-biphenyl]-4-yl)amino)methyl)phenyl)picolinamido)propanoic acid;
3-(5-(2-((2′,4′-dichloro-[1,1′-biphenyl]-4-yl)carbamoyl)-4,5-difluorophenyl)picolinamido)propanoic acid; 3-(5-(4,5-difluoro-2-((4′-fluoro-[1,1′-biphenyl]-4-yl)carbamoyl)phenyl)picolinamido)propanoic acid;
3-(5-(4,5-difluoro-2-((4′-(trifluoromethyl)-[1,1′-biphenyl]-4-yl)carbamoyl)phenyl)picolinamido)propanoic acid;
3-(5-(2-((4′-chloro-[1,1′-biphenyl]-4-yl)carbamoyl)-4,5-difluorophenyl)picolinamido)propanoic acid;
3-(5-(5-chloro-2-(((4′-chloro-2-cyano-3′-(trifluoromethyl)-[1,1′-biphenyl]-4-yl)amino)methyl)phenyl)picolinamido)propanoic acid;
3-(5-(5-chloro-2-(((2′,4′-dichloro-2-cyano-[1,1′-biphenyl]-4-yl)amino)methyl)phenyl)picolinamido)propanoic acid;
3-(5-(5-chloro-2-(((2-cyano-4′-fluoro-3′-(trifluoromethyl)-[1,1′-biphenyl]-4-yl)amino)methyl)phenyl)picolinamido)propanoic acid;
3-(5-(5-chloro-2-(((2′-chloro-2-cyano-3′-(trifluoromethyl)-[1,1′-biphenyl]-4-yl)amino)methyl)phenyl)picolinamido)propanoic acid;
3-(5-(2-(((2-chloro-4′-fluoro-3′-(trifluoromethyl)-[1,1′-biphenyl]-4-yl)amino)methyl)-4,5-difluorophenyl)picolinamido)propanoic acid;
3-(5-(2-(((2,4′-dichloro-[1,1′-biphenyl]-4-yl)amino)methyl)-4,5-difluorophenyl)picolinamido)propanoic acid;
3-(5-(2-(((2,4′-dichloro-2′-fluoro-[1,1′-biphenyl]-4-yl)amino)methyl)-4,5-difluorophenyl)picolinamido)propanoic acid;
3-(5-(2-(((2-chloro-2′-fluoro-4′-methyl-[1,1′-biphenyl]-4-yl)amino)methyl)-4,5-difluorophenyl)picolinamido)propanoic acid;
3-(5-(2-(((2,4′-dichloro-3′-(trifluoromethyl)-[1,1′-biphenyl]-4-yl)amino)methyl)-4,5-difluorophenyl)picolinamido)propanoic acid;
3-(5-(2-(((2,2′-dichloro-3′-(trifluoromethyl)-[1,1′-biphenyl]-4-yl)amino)methyl)-4,5-difluorophenyl)picolinamido)propanoic acid;
3-(5-(4,5-difluoro-2-(((2,2′,4′-trichloro-[1,1′-biphenyl]-4-yl)amino)methyl)phenyl)picolinamido)propanoic acid;
3-(5-(2-(((2-cyano-4′-fluoro-3′-(trifluoromethyl)-[1,1′-biphenyl]-4-yl)amino)methyl)-4,5-difluorophenyl)picolinamido)propanoic acid;
3-(5-(2-(((4′-chloro-2-cyano-[1,1′-biphenyl]-4-yl)amino)methyl)-4,5-difluorophenyl)picolinamido)propanoic acid;
3-(5-(2-(((4′-chloro-2-cyano-2′-fluoro-[1,1′-biphenyl]-4-yl)amino)methyl)-4,5-difluorophenyl)picolinamido)propanoic acid;
3-(5-(2-(((2-cyano-2′-fluoro-4′-methyl-[1,1′-biphenyl]-4-yl)amino)methyl)-4,5-difluorophenyl)picolinamido)propanoic acid;
3-(5-(2-(((4′-chloro-2-cyano-3′-(trifluoromethyl)-[1,1′-biphenyl]-4-yl)amino)methyl)-4,5-difluorophenyl)picolinamido)propanoic acid;
3-(5-(2-(((2′,4′-dichloro-2-cyano-[1,1′-biphenyl]-4-yl)amino)methyl)-4,5-difluorophenyl)picolinamido)propanoic acid;
3-(5-(2-(((2-cyano-4′-fluoro-3′-methyl-[1,1′-biphenyl]-4-yl)amino)methyl)-4,5-difluorophenyl)picolinamido)propanoic acid;
3-(5-(2-(((2-cyano-3′-fluoro-5′-(trifluoromethyl)-[1,1′-biphenyl]-4-yl)amino)methyl)-4,5-difluorophenyl)picolinamido)propanoic acid;
3-(5-(2-(((4′-chloro-[1,1′-biphenyl]-4-yl)amino)methyl)-4,5-difluorophenyl)picolinamido)propanoic acid;
3-(5-(2-(((2′,4′-dichloro-[1,1′-biphenyl]-4-yl)amino)methyl)-4,5-difluorophenyl)picolinamido)propanoic acid;
3-(5-(2-(((4′-chloro-2′-methyl-2-(trifluoromethyl)-[1,1′-biphenyl]-4-yl)amino)methyl)-5-(trifluoromethyl)phenyl)picolinamido)propanoic acid;
3-(5-(2-(((2′-methyl-2,4′-bis(trifluoromethyl)-[1,1′-biphenyl]-4-yl)amino)methyl)-5-(trifluoromethyl)phenyl)picolinamido)propanoic acid;
3-(5-(2-(((4′-fluoro-2,3′-bis(trifluoromethyl)-[1,1′-biphenyl]-4-yl)amino)methyl)-5-(trifluoromethyl)phenyl)picolinamido)propanoic acid;
3-(5-(2-(((2′,4′-dichloro-2-(trifluoromethyl)-[1,1′-biphenyl]-4-yl)amino)methyl)-5-(trifluoromethyl)phenyl)picolinamido)propanoic acid;
3-(5-(2-(((4′-chloro-2-(trifluoromethyl)-[1,1′-biphenyl]-4-yl)amino)methyl)-5-(trifluoromethyl)phenyl)picolinamido)propanoic acid;
3-(5-(2-(((4′-(tert-butyl)-2-(trifluoromethyl)-[1,1′-biphenyl]-4-yl)amino)methyl)-5-(trifluoromethyl)phenyl)picolinamido)propanoic acid;
3-(5-(2-(((2′,4′-difluoro-2-(trifluoromethyl)-[1,1′-biphenyl]-4-yl)amino)methyl)-5-(trifluoromethyl)phenyl)picolinamido)propanoic acid;
3-(5-(2-(((4′-chloro-2′-fluoro-2-(trifluoromethyl)-[1,1′-biphenyl]-4-yl)amino)methyl)-5-trifluoromethyl)phenyl)picolinamido)propanoic acid;
3-(5-(2-(((2,4′-bis(trifluoromethyl)-[1,1′-biphenyl]-4-yl)amino)methyl)-5-(trifluoromethyl)phenyl)picolinamido)propanoic acid;
3-(5-(2-(((2′-chloro-2,4′-bis(trifluoromethyl)-[1,1′-biphenyl]-4-yl)amino)methyl)-5-(trifluoromethyl)phenyl)picolinamido)propanoic acid;
3-(5-(5-chloro-2-(((3′-(trifluoromethoxy)-[1,1′-biphenyl]-4-yl)amino)methyl)phenyl)picolinamido)propanoic acid;
3-(5-(5-chloro-2-(((3′-(trifluoromethyl)-[1,1′-biphenyl]-4-yl)amino)methyl)phenyl)picolinamido)propanoic acid;
3-(5-(5-chloro-2-(((2′-(trifluoromethyl)-[1,1′-biphenyl]-4-yl)amino)methyl)phenyl)picolinamido)propanoic acid;
3-(5-(5-chloro-2-(((2′-(trifluoromethoxy)-[1,1′-biphenyl]-4-yl)amino)methyl)phenyl)picolinamido)propanoic acid;
3-(5-(5-chloro-2-(((2′-fluoro-[1,1′-biphenyl]-4-yl)amino)methyl)phenyl)picolinamido)propanoic acid;
3-(5-(5-chloro-2-(((3′-fluoro-[1,1′-biphenyl]-4-yl)amino)methyl)phenyl)picolinamido)propanoic acid;
3-(5-(5-chloro-2-(((2′-chloro-[1,1′-biphenyl]-4-yl)amino)methyl)phenyl)picolinamido)propanoic acid;
3-(5-(5-chloro-2-(((3′,4′-difluoro-[1,1′-biphenyl]-4-yl)amino)methyl)phenyl)picolinamido)propanoic acid;
3-(5-(5-chloro-2-(((3′,5′-difluoro-[1,1′-biphenyl]-4-yl)amino)methyl)phenyl)picolinamido)propanoic acid;
3-(5-(5-chloro-2-(((2,2′-dichloro-4′-fluoro-[1,1′-biphenyl]-4-yl)amino)methyl)phenyl)picolinamido)propanoic acid;
3-(5-(5-chloro-2-(((2-chloro-4′-fluoro-3′-methyl-[1,1′-biphenyl]-4-yl)amino)methyl)phenyl)picolinamido)propanoic acid;
3-(5-(5-chloro-2-(((2-chloro-4′-(trifluoromethoxy)-[1,1′-biphenyl]-4-yl)amino)methyl)phenyl)picolinamido)propanoic acid;
3-(5-(5-chloro-2-(((2′-chloro-C-fluoro-[1,1′-biphenyl]-4-yl)amino)methyl)phenyl)picolinamido)propanoic acid;
3-(5-(5-chloro-2-(((2-chloro-4′-fluoro-2′-(trifluoromethyl)-[1,1′-biphenyl]-4-yl)amino)methyl)phenyl)picolinamido)propanoic acid;
3-(5-(5-chloro-2-(((2′-chloro-4′-methoxy-[1,1′-biphenyl]-4-yl)amino)methyl)phenyl)picolinamido)propanoic acid;
3-(5-(5-chloro-2-(((2-chloro-4′-fluoro-3′-(trifluoromethyl)-[1,1′-biphenyl]-4-yl)amino)methyl)phenyl)picolinamido)propanoic acid;
3-(5-(24(4′-chloro-[1,1′-biphenyl]-4-yl)carbamoyl)-5-methylphenyl)picolinamido)propanoic acid;
3-(5-(5-chloro-2-(((4′-fluoro-3′-methyl-[1,1′-biphenyl]-4-yl)amino)methyl)phenyl)picolinamido)propanoic acid;
3-(5-(5-chloro-2-(((2′-chloro-4′-(trifluoromethyl)-[1,1′-biphenyl]-4-yl)amino)methyl)phenyl)picolinamido)propanoic acid;
3-(5-(5-chloro-2-(((4′-chloro-2′-methyl-[1,1′-biphenyl]-4-yl)amino)methyl)phenyl)picolinamido)propanoic acid;
3-(5-(5-chloro-2-(((4′-fluoro-2′-(trifluoromethyl)-[1,1′-biphenyl]-4-yl)amino)methyl)phenyl)picolinamido)propanoic acid;
3-(5-(5-chloro-2-(((3′-chloro-4′-fluoro-[1,1′-biphenyl]-4-yl)amino)methyl)phenyl)picolinamido)propanoic acid;
3-(5-(5-chloro-2-(((4′-fluoro-2′-methyl-[1,1′-biphenyl]-4-yl)amino)methyl)phenyl)picolinamido)propanoic acid;
3-(5-(5-chloro-2-(((2′-chloro-4′-fluoro-[1,1′-biphenyl]-4-yl)amino)methyl)phenyl)picolinamido)propanoic acid;
3-(5-(5-chloro-2-(4′-chloro-2′-methyl-[1,1′-biphenyl]-4-yl)carbamoyl)phenyl)picolinamido)propanoic acid;
3-(5-(2-(4′-chloro-2′-methyl-[1,1′-biphenyl]-4-yl)carbamoyl)-5-(trifluoromethyl)phenyl)picolinamido)propanoic acid;
3-(5-(5-chloro-2-(1-(4′-fluoro-[1,1′-biphenyl]-4-yl)amino)ethyl)phenyl)picolinamido)propanoic acid;
3-(5-(2-(((4′-acetyl-[1,1′-biphenyl]-4-yl)amino)methyl)-5-chlorophenyl)picolinamido)propanoic acid;
3-(5-(2-(((4′-fluoro-[1,1′-biphenyl]-4-yl)amino)methyl)-5-(prop-1-en-2-yl)phenyl)picolinamido)propanoic acid;
3-(5-(2-(((4′-fluoro-[1,1′-biphenyl]-4-yl)amino)methyl)-5-isopropylphenyl)picolinamido)propanoic acid;
3-(5-(2-(((2,4′-dichloro-[1,1′-biphenyl]-4-yl)amino)methyl)-3-methoxy-5-(trifluoromethyl)phenyl)picolinamido)propanoic acid;
3-(5-(3-methoxy-2-(((2,4′,6-trichloro-[1,1′-biphenyl]-4-yl)amino)methyl)-5-(trifluoromethyl)phenyl)picolinamido)propanoic acid;
3-(5-(2-(((2′-chloro-4′-(trifluoromethyl)-[1,1′-biphenyl]-4-yl)amino)methyl)-3-methoxy-5-(trifluoromethyl)phenyl)picolinamido)propanoic acid;
3-(5-(2-(((2,4′-dichloro-[1,1′-biphenyl]-4-yl)amino)methyl)-3-methoxy-5-(trifluoromethyl)phenyl)picolinamido)propanoic acid;
3-(5-(2-(((2-chloro-4′-(trifluoromethyl-[1,1′-biphenyl]-4-yl)amino)methyl)-3-methyl-5-(trifluoromethyl)phenyl)picolinamido)propanoic acid;
3-(5-(2-(((2,4′-dichloro-[1,1′-biphenyl]-4-yl)amino)methyl)-3-hydroxy-5-(trifluoromethyl)phenyl)picolinamido)propanoic acid;
3-(5-(2-(((2,4′-dichloro-2′-methyl-[1,1′-biphenyl]-4-yl)amino)methyl)-3-methyl-5-(trifluoromethyl)phenyl)picolinamido)propanoic acid;
3-(5-(2-(((2-chloro-2′-methyl-4′-(trifluoromethyl)-[1,1′-biphenyl]-4-yl)amino)methyl)-3-methyl-5-(trifluoromethyl)phenyl)picolinamido)propanoic acid;
3-(5-(2-(((2-chloro-2′-methyl-4′-(trifluoromethyl41,1′-biphenyll-4-yl)amino)methyl)-3-isopropyl-5-trifluoromethyl)phenyl)picolinamido)propanoic acid;
3-(5-(2-(((4′-(tert-butyl)-2-chloro-[1,1′-biphenyl]-4-yl)amino)methyl)-5-chlorophenyl)picolinamido)propanoic acid;
3-(5-(5-chloro-2-(((2,4′-dichloro-3′-(trifluoromethyl)-[1,1′-biphenyl]-4-yl)amino)methyl)phenyl)picolinamido)propanoic acid;
3-(5-(5-chloro-2-(((2-chloro-3′-(trifluoromethyl)-[1,1′-biphenyl]-4-yl)amino)methyl)phenyl)picolinamido)propanoic acid;
3-(5-(5-chloro-2-(((2,3′-dichloro-4′-fluoro-[1,1′-biphenyl]-4-yl)amino)methyl)phenyl)picolinamido)propanoic acid;
3-(5-(5-chloro-2-(((2-chloro-4′-methyl-[1,1′-biphenyl]-4-yl)amino)methyl)phenyl)picolinamido)propanoic acid;
3-(5-(4-(((2′,4′-dichloro-[1,1′-biphenyl]-4-yl)amino)methyl)-[1,1′-biphenyl]-3-yl)picolinamido)propanoic acid;
3-(5-(4-(((4′-fluoro-[1,1′-biphenyl]-4-yl)amino)methyl)-2′,3′,4′,5′-tetrahydro-[1,1′-biphenyl]-3-yl)picolinamido)propanoic acid;
3-(5-(5-cyclohexyl-2-(((4′-fluoro-[1,1′-biphenyl]-4-yl)amino)methyl)phenyl)picolinamido)propanoic acid;
3-(5-(4-(((2′,4′-dichloro-[1,1′-biphenyl]-4-yl)amino)methyl)-2′,3′,4′,5′-tetrahydro-[1,1′-biphenyl]-3-l)picolinamido)propanoic acid;
3-(5-(2-(((2-chloro-2′-methyl-4′-(trifluoromethyl)-[1,1′-biphenyl]-4-yl)amino)methyl)-3-cyclopropyl-5- (trifluoromethyl)phenyl)picolinamido)propanoic acid;
3-(5-(3-cyclopropyl-2-(((2,4′-dichloro-2′-methyl-[1,1′-biphenyl]-4-yl)amino)methyl)-5-(trifluoromethyl)phenyl)picolinamido)propanoic acid;
3-(5-(2-(((2-chloro-2′-methyl-4′-(trifluoromethyl)-[1,1′-biphenyl]-4-yl)amino)methyl)-5-(trifluoromethyl)-[1,1′-biphenyl]-3-yl)picolinamido)propanoic acid;
3-(5-(2-(((4′-chloro-2-methoxy-2′-methyl-[1,1′-biphenyl]-4-yl)amino)methyl)-3-cyclopropyl-5-trifluoromethyl)phenyl)picolinamido)propanoic acid;
3-(5-(3-cyclopropyl-2-(((2-methoxy-2′-methyl-4′-(trifluoromethyl)-[1,1′-biphenyl]-4-yl)amino)methyl)-5- (trifluoromethyl)phenyl)picolinamido)propanoic acid
N-(4-{(1S)-[1(5-chloro-7-cyano-1-benzothien-3-yl)(4-chlorophenyl)methyl]butyl}benzoyl)-β-alanine;
N-[4-((1S)-1-{(4-chlorophenyl)[5-fluoro-7-(1-methyl-1H-pyrazol-5-yl)-1-benzothien-3-yl]methyl}pentyl)benzoyl]-β-alanine;
4-{(1S)-1-[(7-bromo-5-methyl-1-benzothien-3-yl)(4-chlorophenyl)methyl]butyl}-N-1H-tetrazol-5-ylbenzamide;
4-{(1S)-1-[(7-bromo-5-methyl-1-benzothien-3-yl)(4-chlorophenyl)methyl]butyl}-N-(2H-tetrazol-5-ylmethyl)benzamide;
N-(4-{(1S)-1-[1-benzothien-3-yl(4-chlorophenyl)methyl]butyl}benzoyl)-β-alanine;
N-(4-{(1S)-1-[(5-chloro-7-methyl-1-benzothien-3-yl)(4-chlorophenyl)methyl]butyl}benzoyl)-β-alanine;
N-(4-{(1S)-1-[(5-chloro-7-bromo-1-benzothien-3-yl)(4-chlorophenyl)methyl]butyl}benzoyl)-β-alanine;
N-(4-{(1S)-1-[(2-methyl-1-benzothien-3-yl)(4-chlorophenyl)methyl]butyl}benzoyl)-β-alanine;
N-(4-{(1S)-1-[(5-fluoro-7-chloro-1-benzothien-3-yl)(4-chlorophenyl)methyl]butyl}benzoyl)-β-alanine;
N-(4-{(1S)-1-[-benzothien-3-yl(4-methoxyphenyl)methyl]butyl}benzoyl)-β-alanine;
N-(4-{(1S)-1-[(5-chloro-1-benzothien-3-yl)(3,4-dichlorophenyl)methyl]butyl}benzoyl)-β-alanine;
N-(4-{(1S)-1-[(5-chloro-7-bromo-1-benzothien-3-yl)(3,4-dichlorophenyl)methyl]butyl}benzoyl)-(β-alanine;
N-(4-{(1S)-1-[(5-fluoro-7-cyano-1-benzothien-3-yl)(4-chlorophenyl)methyl]butyl}benzoyl)-β-alanine;
N-(4-{(1S)-1-[(5-methyl-7-cyano-1-benzothien-3-yl)(4-chlorophenyl)methyl]butyl}benzoyl)-β-alanine;
N-(4-{(1S)-1-[(5-trifluoromethyl-7-cyano-1-benzothien-3-yl)(4-chlorophenyl)methyl]butyl}benzoyl)-β-alanine;
N-(4-{(1S)-1-[(5,6-difluoro-7-cyano-1-benzothien-3-yl)(4-chlorophenyl)methyl]butyl}benzoyl)-β-alanine;
N-(4-{(1S)-1-[(4,5-difluoro-7-cyano-1-benzothien-3-yl)(4-chlorophenyl)methyl]butyl}benzoyl)-β-alanine;
N-(4-{(1S)-1-[(2-methyl-5-fluoro-7-cyano-1-benzothien-3-yl)(4-chlorophenyl)methyl]butyl}benzoyl)-β-alanine;
N-(4-{(1S)-1-[(2-methyl-5-chloro-7-cyano-1-benzothien-3-yl)(4-chlorophenyl)methyl]butyl}benzoyl)-β-alanine;
N-(4-{(1S)-1-[(5-trifluoromethyl-7-cyano-1-benzothien-3-yl)(3,5-difluorophenyl)methyl]butyl}benzoyl)-β-alanine;
N-(4-{(1S)-1-[(5-chloro-7-cyano-1-benzothien-3-yl)(3,4-dichlorophenyl)methyl]butyl}benzoyl)-β-alanine;
N-(4-{(1S)-1-[(5-fluoro-7-cyano-1-benzothien-3-yl)(4-trifluoromethoxyphenyl)methyl]butyl}benzoyl)-β-alanine;
N-(4-{(1S)-1-[(5-methyl-7-cyano-1-benzothien-3-yl)(4-trifluoromethoxyphenyl)methyl]butyl}benzoyl)-β-alanine;
N-(4-{(1S)-1-[(5-trifluoromethyl-7-cyano-1-benzothien-3-yl)(4-trifluoromethoxyphenyl)methyl]butyl}benzoyl)-β-alanine;
N-(4-{(1S)-1-[(5-chloro-7-cyano-1-benzothien-3-yl)(4-trifluoromethoxyphenyl)methyl]butyl}benzoyl)-β-alanine;
N-(4-{(1S)-1-[(5,6-difluoro-7-cyano-1-benzothien-3-yl)(4-trifluoromethoxyphenyl)methyl]butyl}benzoyl)-β-alanine;
N-(4-{(1S)-1-[(5-chloro-7-cyano-1-benzothien-3-yl)(4-chlorophenyl)methyl]-4,4,4-trifluorobutyl}benzoyl)-β-alanine;
N-(4-{(1S)-1-[(5-methyl-7-cyano-1-benzothien-3-yl)(4-chlorophenyl)methyl]-4,4,4-trifluorobutyl}benzoyl)-β-alanine;
N-(4-{(1S)-1-[(5-chloro-7-cyano-1-benzothien-3-yl)(4-chlorophenyl)methyl]pentyl}benzoyl)-β-alanine;
N-(4-{(1S)-1-[(5-fluoro-7-cyano-1-benzothien-3-yl)(4-chlorophenyl)methyl]pentyl}benzoyl)-β-alanine;
N-(4-{(1S)-1-[(5-trifluoromethyl-7-cyano-1-benzothien-3-yl)(4-chlorophenyl)methyl]pentyl}benzoyl)-β-alanine;
N-(4-{(1S)-1-[(5-chloro-7-cyano-1-benzothien-3-yl)(4-chlorophenyl)methyl]-4-methylbutyl}benzoyl)-β-alanine;
N-(4-{(1S)-1-[(5-fluoro-7-cyano-1-benzothien-3-yl)(4-chlorophenyl)methyl]-4-methylbutyl}benzoyl)-β-alanine;
N-(4-{(1S)-1-[(5-chloro-7-cyano-1-benzothien-3-yl)(4-chlorophenyl)methyl]propyl}benzoyl)-β-alanine;
N-[4-((1S)-1-{(4-chlorophenyl)[5-chloro-7-(1-methyl-1H-pyrazol-4-yl)-1-benzothien-3-yl]methyl}butyl)benzoyl]-β-alanine;
N-[4-((1S)-1-{(4-trifluoromethoxyphenyl)[5-fluoro-7-(1H-pyrazol-3-yl)-1-benzothien-3-yl]methyl}butyl)benzoyl]-β-alanine;
N-[4-((1S)-1-{(4-chlorophenyl)[5-fluoro-7-(1H-pyrazol-3-yl)-1-benzothien-3-yl]methyl}pentyl)benzoyl]-β-alanine;
N-[4-((1S)-1-{(4-chlorophenyl)[5-chloro-7-(1H-isoxazol-4-yl)-1-benzothien-3-yl]methyl}butyl)benzoyl]-β-alanine;
(2S)-3-[(4-{(1S)-1-[(5-methyl-7-bromo-1-benzothiophen-3-yl)(4-chlorophenyl)methyl]butyl}benzoyl)amino]-2-hydroxypropanoic acid;
3-[(4-{(1S)-1-[(5-methyl-7-bromo-1-benzothiophen-3-yl)(4-chlorophenyl)methyl]butyl}benzoyl)amino]-2,2-difluoropropanoic acid;
3-[(4-{(1S)-1-[(5-methyl-7-bromo-1-benzothiophen-3-yl)(4-chlorophenyl)methyl]butyl}benzoyl)amino]-2-methylpropanoic acid;
3-[(4-{(1S)-1-[(5-methyl-7-bromo-1-benzothiophen-3-yl)(4-chlorophenyl)methyl]butyl}benzoyl)amino]-3-methylpropanoic acid;
2-[(4-{(1S)-1-[(5-methyl-7-bromo-1-benzothiophen-3-yl)(4-chlorophenyl)methyl]butyl}benzoyl)amino]ethanesulfonic acid;
3-[(4-{(1S)-1-[(5-methyl-7-bromo-1-benzothiophen-3-yl)(4-chlorophenyl)methyl]butyl}benzoyl)amino]propanoic amide;
N-[4-((1S)-1-{(4-chlorophenyl)[3-methyl-1-benzothien-2-yl]methyl}butyl)benzoyl]-β-alanine;
N-[4-((1S)-1-{(4-chlorophenyl)[3-methyl-5-chloro-1-benzothien-2-yl]methyl}butyl)benzoyl]-β-alanine;
N-[4((1S)-1-{(4-chlorophenyl)[3-methyl-7-chloro-1-benzothien-2-yl]methyl}butyl)benzoyl]-β-alanine;
N-[4((1S)-1-{(4-methoxyphenyl)[3-methyl-5-chloro-1-benzothien-2-yl]methyl}butyl)benzoyl]-β-alanine;
N-[4-(1-{1-(4-chlorophenyl)-2-oxo-2-[4-(trifluoromethyl)-1,3-dihydro-2H-isoindo1-2-yl]ethyl}butyl)benzoyl]-β-alanine;
N-(4-{1-[1-(4-chlorophenyl)-2-(3,4-dihydroquinolin-1(2H)-yl)-2-oxoethyl]butyl }benzoyl)-β-alanine;
N-(4-{1-[1-(4-chlorophenyl)-2-(2,3-dihydro-1H-indol-1-yl)-2-oxoethyl]butyl}benzoyl)-β-alanine;
N-[4-(1-{1-(4-chlorophenyl)-2-[5-(2-methoxypyridin-3-yl)-1,3-dihydro-2H-isoindo12-yl]-2-oxoethyl}butyl)benzoyl]-β-alanine;
N-[4-(1-{1-(4-chlorophenyl)-2-[5-(2-methoxypyridin-3-yl)-1,3-dihydro-2H-isoindol-2-yl]-2-oxoethyl}butyl)benzoyl]-β-alanine;
N-(4-{(1R)-1-[(R)-(4-chlorophenyl)(3,4-dichlorophenoxy)methyl]butyl}benzoyl)-β-alanine;
N-(4-{(1R)-1-[(R)-(3-chloro-5-cyanophenoxy)(4-chlorophenyl)methyl]butyl}benzoyl)-β-alanine;
N-[4-((1R)-1-{(R)-(4-chlorophenyl)[(2-cyano-4′-methylbiphenyl-4-yl)oxy]methyl}butyl)benzoyl]-β-alanine;
N-[4-((1R)-1-{(R)-(4-chlorophenyl)[(3-cyano-4′-methylbiphenyl-4-yl)oxy]methyl}butyl)benzoyl]-β-alanine;
N-(4-{(1R)-1-[(R)-[(6-chloro-8-methylquinolin-4-yl)oxy](4-chlorophenyl)methyl]butyl}benzoyl)-β-alanine;
N-[4-((1R)-1-{(R)-(4-chlorophenyl)[(7-methylquinolin-4-yl)oxy]methyl}butyl)benzoyl]-β-alanine;
N-[4-((1R)-1-{(R)-(4-chlorophenyl)[(7-chloroquinolin-4-yl)oxy]methyl}butyl)benzoyl]-β-alanine;
N-(4-{(1R)-1-[(R)-[(2-trifluoromethylquinolin-4-yl)oxy](4-chlorophenyl)methyl]butyl}benzoyl)-β-alanine;
N-[4-((1R)-1-{(R)-(4-chlorophenyl)[(7-cyanoquinolin-4-yl)oxy]methyl}butyl)benzoyl]-β-alanine;
N-{4-[(1R)-1-((R)-(4-chlorophenyl){[4-(trifluoromethoxy)benzyl]oxy}methyl)butyl]benzoyl}-β-alanine;
N-(4-{(1R)-1-[(R)-(4-chlorophenyl)(2-naphthylmethoxy)methyl]butyl}benzoyl)-(3-alanine;
N-(4-{(1R)-1-[(R)-(biphenyl-4-ylmethoxy)(4-chlorophenyl)methyl]butyl}benzoyl)-β-alanine;
N-(4-{(1R)-1-[(R)-(3-chloro-5-cyanophenoxy)(4-chlorophenyl)methyl]-4,4,4-trifluorobutyl}benzoyl)-β-alanine;
N-[4-((1R)-1-{(R)-(4-chlorophenyl)[3-cyano-5-(trifluoromethyl)phenoxy]methyl}butyl)benzoyl]-β-alanine;
N-[4-((1R)-1-{(R)-(4-trifluoromethoxyphenyl)[3-cyano-5-(trifluoromethyl)phenoxy]methyl}butyl)benzoyl]-β-alanine;
N-(4-{(1R)-1-[(R)-[4-chloro-3-cyano-5-(trifluoromethyl)phenoxy](4-chlorophenyl)methyl]butyl}benzoyl)-β-alanine;
N-[4-(1-{1-(4-chlorophenyl)-2-[(6-methoxy-2-naphthyl)oxy]ethyl}butyl)benzoyl]-β-alanine;
N-[4-(1-{1-(4-chlorophenyl)-2-[(4′-methoxybiphenyl-4-yl)oxy]ethyl}butyl)benzoyl]-β-alanine;
N-[4-(1-{1-(4-chlorophenyl)-2-[(trifluoromethoxy)phenoxy]ethyl}butyl)benzoyl]-β-alanine;
N-4-[1-{1-(4-chlorophenyl)-2-{[4-(trifluoromethoxy)benzyl]oxy}ethyl)butyl]benzoyl}-β-alanine;
N-(4-{1-[2-(biphenyl-4-ylmethoxy)-1-(4-chlorophenyl)ethyl]butyl}benzoyl)-β-alanine;
N-(4-{1[2-(1,3-benzothiazol-2-ylmethoxy)-1-(4-chlorophenyl)ethyl]butyl}benzoyl)-β-alanine;
N-(4-{1-(5-Chloro-7-Methyl-1H-Indazol-3-yl)(4-Chlorophenyl)Methyl]Butyl}Benzoyl)-β-Alanine;
N-(4-{1-[[7-Chloro-1-Methyl-5-(Trifluoromethyl)-1H-Indazol-3-yl](4-Chlorophenyl)Methyl]Butyl}Benzoyl)-β-Alanine;
N-[4-(1-{(4-Chlorophenyl)[7-Chloro-2-Phenyl-5-(Trifluoromethyl)-2H-Indazol-3-yl]Methyl}Butyl)Benzoyl]-β-Alanine;
N-[4-(1-{(4-Chlorophenyl)[7-Chloro-2-Methyl-5-(Trifluoromethyl)-2H-Indazol-3-yl]Methyl}Butyl)Benzoyl]-β-Alanine;
N-[4-(1-{(4-chlorophenyl)[5-(trifluoromethyl)-1H-indazol-3-yl]methyl}butyl)benzoyl]-β-alanine;
N-[4-(1-{(4-chlorophenyl)[7-chloro-5-(trifluoromethyl)-1 H-indazol-3 -yl]methyl}butyl)benzoyl]-β-alanine;
N-[4-(1-{(4-chlorophenyl)[5,7-difluoro-1H-indazol-3-yl]methyl}butyl)benzoyl]-β-alanine;
N-[4-(1-{(4-chlorophenyl)[5,7-dichloro-1H-indazol-3-yl]methyl}butyl)benzoyl]-β-alanine;
N-[4-(1-{(4-chlorophenyl)[1-methyl-5-(trifluoromethyl)-1H-indazol-3-yl]methyl}butyl)benzoyl]-β-alanine;
N-[4-(1-{(4-chlorophenyl)[1-methyl-5,7-difluoro-1H-indazol-3-yl]methyl}butyl)benzoyl]-β-alanine;
N-[4-(1-{(4-chlorophenyl)[1,7-dimethyl-5-chloro-1H-indazol-3-yl]methyl}butyl)benzoyl]-β-alanine;
N-[4-(1-{(4-chlorophenyl)[1-methyl-5,7-dichloro-1H-indazol-3-yl]methyl}butyl)benzoyl]-β-alanine;
N-[4-(1-{(4-chlorophenyl)[2-methyl-5-(trifluoromethyl)-2H-indazol-3-yl]methyl}butyl)benzoyl]-β-alanine;
N-[4-(1-{(4-chlorophenyl)[7-chloro-2-ethyl-5-(trifluoromethyl)-2H-indazol-3-yl]methyl}butyl)benzoyl]-β-alanine;
N-[4-(1-{(4-chlorophenyl)[7-chloro-2-n-propyl-5-(trifluoromethyl)-2H-indazol-3-yl]methyl}butyl)benzoyl]-β-alanine;
N-[4-(1-{(4-chlorophenyl)[7-chloro-2-isopropyl-5-(trifluoromethyl)-2H-indazol-3-yl]methyl}butyl)benzoyl]-β-alanine;
N-[4-(1-{(4-chlorophenyl)[7-chloro-2-(2,2,2-trifluoroethyl)-5-(trifluoromethyl)-2H-indazol-3-yl]methyl}butyl)benzoyl]-β-alanine;
N-[4-(1-{(4-chlorophenyl)[2-methyl-5,7-difluoro-2H-indazol-3-yl]methyl}butyl)benzoyl]-β-alanine;
N-[4-(1-{(4-chlorophenyl)[2-methyl-5,7-dichloro-2H-indazol-3-yl]methyl}butyl)benzoyl]-β-alanine;
N-[4-(1-{(4-chlorophenyl)[5-chloro-2,7-dimethyl-2H-indazol-3-yl]methyl}butyl)benzoyl]-β-alanine;
N-[4-(1-{(4-Chlorophenyl)[7-Fluoro-5-(4-Methylphenyl)-1H-Indazol-3-yl]Methyl}Butyl)Benzoyl]-β-Alanine;
N-(4-{1-[(4-Chlorophenyl)(5-Cyano-7-Fluoro-1H-Indazol-3-yl)Methyl]Butyl}Benzoyl)-β-Alanine;
N-(4-{(1S)-1-[(R)-(4-Chlorophenyl)(7-Fluoro-5-Methyl-1H-Indazol-3-yl)Methyl]Butyl}Benzoyl)-β-Alanine;
3-({4-[1-[4-(Benzyloxy)phenyl]-4-(trifluoromethyl)cyclohexyl]benzoyl}amino)propanoic acid;
3-({4-[1-{4-[(3-Fluorobenzyl)oxy]phenyl}-4-(trifluoromethyl)cyclohexyl]benzoyl}amino)propanoic acid;
3-({-4-[1-[4′-(trifluoromethoxy)biphenyl-4-yl]-4-(trifluoromethyl)cyclohexyl]benzoyl}amino)propanoic acid;
3-({4-[1-[4′-Chloro-3′-(trifluoromethyl)biphenyl-4-yl]-4-trifluoromethyl)cyclohexyl]benzoyl}amino)propanoic acid;
3-({4-[1-(4-[(4-Fluorobenzyl)oxy]phenyl)-4-(trifluoromethyl)cyclohexyl]benzoyl}amino)propanoic acid;
3-({4-[1-[4-(3,4-Dihydroquinolin-1(2H)-yl)phenyl]-4-trifluoromethyl)cyclohexyl]benzoyl}amino)propanoic acid;
N-({4-[1-{3,5-Dichloro-4-[(cyclohexylmethyl)oxy]phenyl}-4-(trifluoromethyl)cyclohexyl]phenyl}carbonyl)-β-alanine;
3-({4-[1-[2-(4-tert-Butylphenyl)-1-benzofuran-5-yl]-4-(trifluoromethyl)cyclohexyl]benzoyl}amino)propanoic acid;
N-{4-[1-(7-Chloro-2-propyl-1-benzofuran-5-yl)-4-(trifluoromethyl)cyclohexyl]benzoyl}-β-alanine;
N-(4-{2-[3-bromo-4-(cyclohexylmethoxy)phenyl]bicyclo[2.2.1]hept-2-yl}benzoyl)-β-alanine;
N-(4-{2-[3,5-dibromo-4-(cyclohexylmethoxy)phenyl]bicyclo[2.2.1]hept-2-yl}benzoyl)-β-alanine; N-(4-{2-[3-cyano-4-(cyclohexylmethoxy)phenyl]bicyclo[2.2.1]hept-2-yl}benzoyl)-β-alanine;
3-({4-[1-(2-Oxo-3,4-dipropyl-2H-chromen-7-yl)-4-(trifluoromethyl)cyclohexyl]benzoyl}amino)propanoic acid;
3-({4[1-(2-Propyl-3,4-dihydro-2H-chromen-6-yl)-4-(trifluoromethyl)cyclohexyl]benzoyl}amino)propanoic acid;
N-{4-[1-{4-[1-Methyl-5 -(trifluoromethyl)-1H-benzimidazol-2-yl]phenyl}-4(trifluoromethyl)cyclohexyl]benzoyl}-β-alanine;
N-{4-[1-[2-(4-Fluorophenyl)-1,3-benzoxazol-5-yl]-4-(trifluoromethyl)cyclohexyl]benzoyl}-β-alanine;
N-{4-[1-[2-(4-Fluorophenyl)-1H-indol-6-yl]-4-(trifluoromethyl)cyclohexyl]benzoyl}-β-alanine;
N-(4-{4-(Trifluoromethyl)-1-[7-(trifluoromethyl)quinolin-3-yl]cyclohexyl}benzoyl)-β-alanine;
3-[({4-[1-[7-Chloro-5-fluoro-1-benzofuran-2-yl)phenyl]-4-(trifluoromethyl)cyclohexyl]phenyl}carbonyl)amino]propanoic acid;
N-(4-{1(1R or S)-1-[6-(3,4-Difluorophenyl)-2-naphthyl]but-3-en-1-yl}benzoyl)-β-alanine;
N-(4-{1(R or S)-1-[6-(3,4-Difluorophenyl-2-naphthyl]-4-methylpentyl}benzoyl)-β-alanine;
N-{4-[(1R or S)-1-(5-Cyano-6-propylthio-2-naphthyl)butyl]benzoyl}-β-alanine;
N-{4-[(1R)-4,4,4-Trifluoro-1-(6-hydroxy-2-naphthyl)butyl]benzoyl}-β-alanine;
N-{4-[(1S)-4,4,4-Trifluoro-1-(6-hydroxy-2-naphthyl)butyl]benzoyl}-β-alanine;
N-(4-{1(1R or S)-1-[6-(Ethylthio)-2-naphthyl]-4,4,4-trifluorobutyl}benzoyl)-β-alanine;
N-{4-[(1R or S)-1-(6-Cyclohexyl-2-naphthyl)-4,4,4-trifluorobutyl]benzoyl}-β-alanine;
N-{4-[(1R or S)-1-(6-Butoxy-5,7-dichloro-2-naphthyl)-4,4,4-trifluorobutyl]benzoyl}-β-alanine;
N-{4-[(1RS)-2-(4-Chlorophenyl)-1-(6-methoxy-2-naphthyl)ethyl]benzoyl}-β-alanine;
N-{4-[(1 (RS)-1-(6-Butoxy-2-naphthyl)-2-phenylethyl]benzoyl}-β-alanine;
N-{4-[(1R or S)-1 -(2-Butoxyquinolin-6-yl)-4,4,4-trifluorobutyl]benzoyl}-β-alanine;
N-(4-{(1R or S)-4,4,4-Trifluoro-1-[2-(trifluoromethyl)quinolin-6-yl]butyl}benzoyl)-β-alanine;
3-{[4-((1(RS)-1-[5-[(2,4-Dichlorobenzyl)oxy]-1-methyl-1H-benzimidazol-2-yl}butyl)benzoyl]amino}propanoic acid;
3-({4-[1(RS)-1-(5-Phenyl-1,3-benzoxazol-2-yl)butyl]benzoyl}amino)propanoic acid;
N-{4-[(1R or S)-1-(6-Butoxy-2-naphthyl)-4-(ethylthio)butyl]benzoyl}-β-alanine;
N-(4-{(1R)-1-[7-(3,4-Difluorophenyl)quinolin-3-yl]-4,4,4-trifluorobutyl}benzoyl)-β-alanine;
N-(4-{(1S)-1-[7-(3,4-Difluorophenyl)quinolin-3-yl]-4,4,4-trifluorobutyl}benzoyl)-β-alanine;
N-(4-{(1S) 1-[(4-Chlorophenyl)(6-Chloro-8-Methylquinolin-4-yl)Methyl]Butyl}Benzoyl)-β-Alanine;
N-(4-{(1S)-1-[(4-Chlorophenyl)(2-Methoxyquinolin-5-yl)Methyl]Butyl}Benzoyl)-β-Alanine;
N-(4-{(1S)-1-[(4-Chlorophenyl)(7-Trifluoromethylquinolin-3-yl)Methyl]Butyl}Benzoyl)-β-Alanine;
N-(4-{(1S)-1-[(4-Chlorophenyl)(6,7-Difluoroquinolin-4-yl)Methyl]Butyl}Benzoyl)-β-alanine;
N-[4-{(1S)-1-(4-Chlorophenyl)[2-(Difluoromethoxy)Quinolin-5-yl]Methyl}Butyl)Benzoyl]-β-Alanine;
N-(4-{(1S)-1-[(4-Chlorophenyl)(2-(1H-Pyrol-2-Yl)Quinolin-5-yl)Methyl]Butyl}Benzoyl)-β-Alanine;
N-(4-{(1S)-1-[(4-Chlorophenyl)(2-(1H-Pyrazol-5-Y1)Quinolin-5-yl)Methyl]Butyl}Benzoyl)-β-Alanine;
N-(4-{1S)-1-[(4-Chlorophenyl)(2-(3,5-Dimethylisoxazol-4-yl)Quinolin-5-yl)Methyl]Butyl}Benzoyl)-β-Alanine;
N-(4-{(1S)-1-[(R)-(4-Chlorophenyl)(8-Fluoro-6-Methylquinazolin-4-yl)Methyl]Butyl}Benzoyl)-β-Alanine;
N-(4-{1-[(4-Chlorophenyl)(4-Ethyl-6-Methoxyquinazolin-2-yl)Methyl]Butyl}Benzoyl)-β-Alanine;
N-[4-{(1S)-1-(4-Cyclopentylphenyl)[7-(Trifluoromethyl)Quinolin-3-yl]Methyl}Butyl)Benzoyl]-β-Alanine;
1-[(trans-4-tert-butylcyclohexyl)({[4-(trifluoromethoxy)phenyl]amino}carbonnyl)amino]-N-1H-tetrazol-5-ylindane-5-carboxannoe;
N-({1-[trans-4-tert-butycyclohexyl)({[4-(trifluoromethoxy)phenyl]amino}carbonyl)amino]-2,3-dihydro-1H-inden-5-yl}carbonyl)-β-alanine;
(2R)-3[({1-[(trans-4-tert-butylcyclohexyl)({[4-(trifluoromethoxy)phenyl]amino}carbonyl)amino]-2,3-dihydro-1H-inden-5-yl}carbonyl)amino]-2-hydroxypropanoic acid;
1-{(trans-4-tert-butylcyclohexyl))[4-(trifluoromethoxy)benzoyl]amino}-N-1H-tetrazol-5-ylindane-5-carboxamide;
1((Trans-4-tert-butylcyclohexy){[4-(trifluoromethoxy)phenyl]acetyl}amino)-N-1H-tetrazol-5-ylindane-5-carboxamide;
1-{(Trans-4-tert-butylcyclohexyl)[(4-isopropylphenyl)acetyl]amino}-N-1H-tetrazol-5-ylindane-5-carboxamide;
6-bromo-4-[(trans-4-tert-butylcyclohexyl)({[4-trifluoromethoxy)phenyl]amino}carbonyl)amino]-N-1H-tetrazol-5 -ylchromane-7-carboxamide;
N-({6-bromo-4-[(trans-4-tert-butylcyclohexyl)({[4-(trofluoroomethoxy)phenyl]amino}carbonyl)amino]-3,4-dihydro-2H-chromen-7-yl}carbonyl)-β-alanine;
N-(4-{1-[(4-chlorophenyl)(5,7-dichloro-1H-indol-3-yl)methyl]butyl}benzoyl)-β-alanine;
N-(4-{1(1S)-1-[(R)-(4-chlorophenyl)(7-fluoro-5-methyl-1H-indol-3-yl)methyl]butyl}benzoyl)-β-alanine;
4-{1-[(3-chloro-4-methoxyphenyl)(5,7-dichloro-1H-indol-3-yl)methyl]pentyl}benzoyl)-β-alanine;
N-(4-{1-[(5,7-dichloro-1H-indol-3-yl)(3,5-dichloro-4-methoxyphenyl)methyl]pentyl}benzoyl)-β-alanine;
N-{4-[2-{3-[(1E)-hex-1-en-1-yl]phenyl}-2-(1H-indol-3-yl)-1-methylethyl]benzoyl}-β-alanine;
N-{4-[2-(3-hexylphenyl)-2-(1H-indol-3-yl)-1-methylethy]benzoyl}-β-alanine; and
N-(4-{1-[(4-chlorophenyl)(5,7 -dichloro-1-methyl-1H-indol-3-yl)methyl]butyl}benzoyl)β-alanine.

Specific glucagon receptor modifiers of the present invention include:
[N-[(4-{(1S)-1-[3-(3,5-dichlorophenyl)-5-(6-methoxynaphthalen-2-yl)-1H-pyrazol-1-yl]ethyl}phenyl) carbonyl]-β-alanine]; and
[N-(4-{(1S)-1-[(R)-(4-chlorophenyl)(7-fluoro-5-methyl-1H-indol-3-yl)methyl]butyl}benzoyl)-β-alanine].

A further embodiment of the present invention is a glucagon receptor antagonist selected from the group consisting of:
(R,S)-3-({5-[1-(4′-Trifluoromethyl-biphenyl-4-yloxy)-propyl]-thiophene-2-carbonyl}-amino)-propionic acid;
(R,S)-3-({5-[1-(4-Trifluoromethyl-biphenyl-4-yloxy)-butyl]-thiophene-2-carbonyl}-amino)-propionic acid;
(R,S)-3-({5-[1-(4-Trifluoromethyl-biphenyl-4-yloxy)-ethyl]-thiophene-2-carbonyl}-amino)-propionic acid;
(R,S)-3-({5-[1-(4′-Trifluoromethyl-biphenyl-4-yloxy)-hexyl]-thiophene-2-carbonyl}-amino)-propionic acid;
(R,S)-3-({5-[Cyclohexyl-(4′-trifluoromethyl-biphenyl-4-yloxy)-methyl]-thiophene-2-carbonyl}-amino)-propionic acid;
(R,S)-3-({5-[2,2-Dimethyl-1-(4′-trifluoromethyl-biphenyl-4-yloxy)-propyl]-thiophene-2-carbonyl}-amino)-propionic acid;
(R,S)-3-({5-[3,3-Dimethyl-1-(4′-trifluoromethyl-biphenyl-4-yloxy)-butyl]-thiophene-2-carbonyl}-amino)-propionic acid;
(R,S)-3-({5-[4-Methyl-1-(4-trifluoromethyl-biphenyl-4-yloxy)-pentyl]-thiophene-2-carbonyl}-amino)-propionic acid;
(R,S)-3-({5-[1-(2,6-Dimethyl-4′-trifluoromethyl-biphenyl-4-yloxy)-2,2-dimethyl-propyl]-thiophene-2-carbonyl}-amino)-propionic acid;
(R,S)-3-({5-[1-(2,6-Dimethyl-4′-trifluoromethyl-biphenyl-4-yloxy)-3,3-dimethyl-butyl]-thiophene-2-carbonyl}-amino)-propionic acid;
(R,S)-3-({5-[3,3-Dimethyl-1-(2-methyl-4′-trifluoromethyl-biphenyl-4-yloxy)-butyl]-thiophene-2-carbonyl}-amino)-propionic acid;
(R,S)-3-({5-[1-(4′-tert-Butyl-2-methyl-biphenyl-4-yloxy)-3,3-dimethyl-butyl]-thiophene-2-carbonyl}-amino)-propionic acid;
(R,S)-3-[(5-{3,3-Dimethyl-1-[6-(4-trifluoromethyl-phenyl)-pyridin-3-yloxy]-butyl}-thiophene-2-carbonyl)-amino]-propionic acid;
(R,S)-3-{(5-{1-[6-(4-tert-Butyl-phenyl)-pyridin-3-yloxy}-3,3-dimethyl-butyl]-thiophene-2-carbonyl)-amino}-propionic acid;
(R,S)-3-({5-[1-(4′-tert-Butyl-2-methyl-biphenyl4-yloxy)-2,2-dimethyl-propyl]-thiophene-2-carbonyl}-amino)-propionic acid;
(R,S)-3-({5-[2,2-Dimethyl-1-(2-methyl-4′-trifluoromethyl-biphenyl-4-yloxy)-propyl]-thiophene-2-carbonyl}-amino)-propionic acid;
(R,S)-3-({5-[1-(4′-tert-Butyl-2-methyl-biphenyl-4-yloxy)-propyl]-thiophene-2-carbonyl}-amino)-propionic acid;
(R,S)-3-({5-[1-(2-Methyl-4′-trifluoromethyl-biphenyl-4-yloxy)-propyl]-thiophene-2-carbonyl}-amino)-propionic acid;
(R,S)-3({5-[1-(2,6-Dimethyl-4′-trifluoromethyl-biphenyl-4-yloxy)-propyl]-thiophene-2-carbonyl}-amino)-propionic acid;
(R,S)-3-({5-[1-(4′-tert-Butyl-2,6-dimethyl-biphenyl-4-yloxy)-propyl]-thiophene-2-carbonyl}-amino)-propionic acid;
(R,S)-3-({5-[1-(2,6-Dimethyl-4′-trifluoromethyl-biphenyl-4-yloxy)-3-methyl-butyl]-thiophene-2-carbonyl}-amino)-propionic acid;
(R,S)-3-({5-[1-(4′-tert-Butyl-2,6-dimethyl-biphenyl-4-yloxy)-2,2-dimethyl-propyl]-thiophene -2-carbonyl}-amino)-propionic acid;
3-{[5-(4′-tert-Butyl-2,6-dimethyl-biphenyl-4-yloxymethyl)-thiophene-2-carbonyl]-amino}-propionic acid;
(R,S)-3-({5-[3-Methyl-1-(2-methyl-4′-trifluoromethyl-biphenyl-4-yloxy)-butyl]-thlophene-2-carbonyl}-amino)-propionic acid;
(R,S)-3 -({5-[1-(4′-tert-Butyl-2-methyl-biphenyl-4-yloxy)-3-methyl-butyl]-thiophene-2-carbonyl}-amino)-propionic acid;
3-({5-[1-(4-Trifluoromethyl-biphenyl-4-yloxy)-ethyl]-thiophene-2-carbonyl}-propionic acid;
3-({5-[1-(4′-Trifluoromethyl-biphenyl-4-yloxy)-propyl]-thiophene-2-carbonyl}-amino)-propionic acid;
3-({5-[1-(4′-Trifluoromethyl-biphenyl-4-yloxy)-butyl]thiophene-2-carbonyl}-amino)-propionic acid;
3-({5-[2,2-Dimethyl-1-(4′-trifluoromethyl-biphenyl-4-yloxy)-propyl]-thiophene-2-carbonyl}-amino)-propionic acid;
3-({5-[3,3-Dimethyl-1-(4′trifluoromethyl-biphenyl-4-yloxy)-butyl]-thiophene-2-carbonyl}-amino)-propionic acid;
3-({5-[2,2-Dimethyl-1-(2-methyl-4′-trifluoromethyl-biphenyl-4-yloxy)-propyl]-thiophene-2-carbonyl}-amino)-propionic acid;
3-({5-[1-(4′-tert-Butyl-2-methyl-biphenyl-4-yloxy)-propyl]-thiophene-2-carbonyl}-amino)-propionic acid;
3-({5-[3,3-Dimethyl-1(2-methyl-4′-trifluoromethyl-biphenyl-4-yloxy)-butyl]-thiophene-2-carbonyl}-amino)-propionic acid;
3-({5-[3,3-Dimethyl-1-(2-methyl-4′-trifluoromethyl-biphenyl-4-yloxy)-butyl]-thiophene-2-carbonyl}-amino)-propionic acid;
3-({5-[1-(2,6-Dimethyl-4′-trifluoromethyl-biphenyl-4-yloxy)-propyl]-thiophene -2-carbonyl}-amino)-propionic acid;
3-({5-[1-(4′-tert-Butyl-2,6-dimethyl-biphenyl-4-yloxy)-propyl]-thiophene-2-carbonyl}-amino)-propionic acid;
(R,S)-3-({5-[2-Methyl-1-(4′-trifluoromethyl-biphenyl-4-yloxy)-propyl]-thiophene-2-carbonyl}-amino)-propionic acid;
(R,S)-3-({5-[2-Methyl-1-(4′-trifluoromethyl-biphenyl-4-yloxy)-propyl]-thiophene-2-carbonyyl}-amino)-propionic acid;
3-({5-[2-Methyl-1-(4′-trifluoromethyl-biphenyl-4-yloxy)-propyl]-thiophene-2-carbonyl}-amino)-propionic acid;
(R,S)-3-({5-[1-(4′-Trifluoromethyl-biphenyl-4-yloxy)-octyl]-thiophene-2-carbonyl}-amino)-propionic acid;
3-({5-[1-4′-Trifluoromethyl-biphenyl-4-yloxy)-octyl]-thiophene-2-carbonyl}-amino)-propionic acid;
(R,S)-3-({5-[1-(2,6-Dimethyl-4′-trifluoromethyl-biphenyl-4-yloxy)-3,3-dimethyl-butyl]-thiophene-2-carbonyl}-amino)-propionic acid;
3-({5-[1-(2,6-Dimethyl-4′-trifluoromethyl-biphenyl-4-yloxy)-3,3-dimethyl-butyl]-thiophene-2-carbonyl}-amino)-propionic acid;
(R,S)-3-({3-Chloro-5-[1-(2,6-dimethyl-4′-trifluoromethyl-biphenyl-4-yloxy)-propyl]-thiophene-2-carbonyl}-amino)-propionic acid;
(R,S)-3-({5-[1-(4′-tert-Butyl-2,6-dimethyl-biphenyl-4-yloxy)-4,4,4-trifluoro-butyl]-thiophene-2-carbonyl}amino)-propionic acid;
(R,S)-3-({5-[1-(2,6-Dimethyl-4′-trifluoromethyl-biphenyl-4-yloxy)-2-methyl-propyl]-thiphene-2-carbonyl}-amino)-propionic acid;
(R,S)-3-({5-[2-Methyl-1-(2-methyl-4′-trifluoromethyl-biphenyl-4-yloxy)-proyl]-thiophene-2-carbonyl}-amino)-propionic acid;
(R,S)-3-({5-[1-(2,6-Dimethyl-4′-trifluoromethyl-biphenyl4-yloxy)-butyl]-thiophene-2-carbonnl}-amino)-propionic acid;
(R,S)-3-({5-[1-(2-Methyl-4′-trifluoromethyl-biphenyi-4-yloxy)-butyl]-thiophene-2-carbonyl}-amino-propionic acid;
(R,S)-3-({5-[1-(4′-tert-Butyl-2,6-dimethyl-biphenyl-4-yloxy)-2-methyl-propyl]-thiophene-2-carbonyl}-amino)-propionic acid;
3-({5-[1-(4′-tert-Butyl-2,6-dimethyl-biphenyl-4-yloxy)-2-methyl-propyl]-thiophene-2-carbonyl}-amino)-propionic acid;
(R,S)-3-({5-[1-(4′-tert-Butyl-2-methyl-biphenyl-4-yloxy)-2-methyl-propyl]-thiophene-carbonyl}-amino)-propionic acid;
3-({5-[1-(4′-tert-Butyl-2-methyl-biphenyl-4-yloxy)-2-methyl-propyl]-thiophene-2-carbonyl}-amino)propionic acid;
(R,S)-3-({5-[1-(4′- Butyl-2,6-dimethyl-biphenyl-4-yloxy)-butyl]-thiophene-2-carbonyl}-amino)-propionic acid;
(R,S)-3-({5-[1-(4′-tert-Butyl-2-methyl-biphenyl-4-yloxy)-butyl]-thiophene-2-carbonyl}-amino)-propionic acid;
(R,S)-3-({5-[1-(2,6-Dimethyl-4′-trifluoromethyl-biphenyl-4-yloxy)-ethyl]thiophene 2-carbonyl}-amino)-propionic acid;
(R,S)-3-({5-[1-(4′-tert-Butyl-2,6-dimethyt-biphenyl-4-yloxy)-ethyl]-thiophene-2-carbonyl}-amino)-propionic acid;
(R,S)-3-({5-[1-(2,6-Dimethyl-4′-trifluoromethyl-biphenyl-4-yloxy)-pentyl]-thiophene-2-carbonyl}-amino)-propionic acid;
(R,S)-3-({5-[1-(2-Methyl-4′-trifluoromethyl-biphenyl-4-yloxy)-pentyl]-thiophene-2-carbonyl}-amino)-propionic acid;
(R,S)-3-({5-[1-(4′-tert-Butyl-2,6-dimethyt-biphenyl-4-yloxy)-pentyl]-thiophene-2-carbonyl}-amino)-propionic acid;
(R,S)-3-({5-[1-(4′-tert-Butyl-2-methyl-biphenyl-4-yloxy)-pentyl]-thiophene-2-carbonyl}-amino)-propionic acid;
3-({5-[2-(2,6-Dimethyl-4′-trifluoromethyl-biphenyl-4-yloxy)-1,1-dimethyl-ethyl]-thiophene-2-carbonyl}-amino)-propionic acid;
3-({5-[4-(2,6-Dimethyl-4′-trifluoromethyl-biphenyl-4-yloxymethyl)-ethyl-propyl]-thiophene-2-carbonyl}-amino)-propionic acid;
3-({5-[1-(2,6-Dimethyl-4′-trifluoromethyl-biphenyl-4-yloxymetheyl)-1propyl-butyl]-thiophene-2-carbonyl}-amino)-propionic acid;
3-({5-[1-Allyl-1-(2,6-dimethyl-4′-trifluoromethyl-biphenyl-4-yloxymethyl)-but-3-enyl]-thiophene-2-carbonyl}-amino)-propionic acid;
3-({5-[1-(2,6-Dimethyl-4′-trifluoromethyl-biphenyl-4-yloxymethyl)-cyclopent-3-enyl]-thiophene-2-carbonyl}-amino)-propionic acid;
3-({5-[1-(2,6-Dimethyl-4′-trifluoromethyl-biphenyl-4-yloxymethyl)-cyclopentyl]-thiophene-2-carbonyl}-amino)-propionic acid;
(R,S)-3-({5-[1(4-Bromo-3,5-dimethyl-phenoxy)-3-methyl-butyl]-thiopene-2-carbonyl}-amino)-propionic acid;
(R,S)-3-({5-[1-(3,5-Dimethyl-phenoxy)-3-methyl-butyl]-thiophene-2-carbonyl}-amino)-propionic acid;
(R,S)-3-({5-[1-(4-Bromo-3,5-dimethyl-phenylsulfanyl)-4,4,4-trifluoro-butyl]-thiophene-2-carbonyl}-amino)-propionic acid;
3-({5-[1-(2,6-Dimethyl-4′-trifluoromethyl-biphenyl-4-yloxy)-2-methyl-propyl]-thiophene-2-carbonyl}-amino)-propionic acid;
(R,S)-3-({5-[1-(2,6-Dimethyl-biphenyl-4-yloxy)-3-methyl-butyl]-thiophene-2-carbonyl}-amino)-propionic acid;
(R,S)-3-({5-[1-(4′-tert-Butyl-2,6-dimethyl-biphenyl-4-yloxy)-3-methyl-butyl]-thiophene-2-carbonyl}-amino)-propionic acid;
(R,S)-3-({5-[1(2,6-Dimethyl-2′-trifluoromethyl-biphenyl-4-yloxy)-3-methyl-butyl]-thiophene-2-carbonyl}-amino)-propionic acid;
(R,S)-3-({5[1-(2,6-Dimethyl-3′-trifluoromethyl-biphenyl-4-yloxy)-3 methyl-butyl]-thiophene-2-carbonyl}-amino)-propionic acid;
(R,S)-3-({5-[1-(4′-Ethyl-2,6-dimethyl-biphenyl-yloxy)-3-methyl-butyl]-thiophene-2-carbonyl}-amino)-propionic acid;
(R,S)-3-({5-[3-Methyl-1-(2,6,4′-trimethyl-biphenyl-4-yloxy)-butyl]-thiophene-2-carbonyl}-amino)-propionic acid;
(R,S)-3-({5-[1-(4′-Isopropyl-2,6-dimethyl-biphenyl-4-yloxy)-3-methyl-butyl]-thiophene,-2-carbonyl}-amino)-propionic acid;
(R,S)-3-({5-[1-(2,6-Dimethyl-4′-pentyl-biphenyl-4-yloxy)-3-methyl-butyl]-thiophene-2-carbonyl}-amino)-propionic acid;
(R,S)-3-({5-[1-(4′-Cyclohexy-2,6-dimethyl-biphenyl-4-yloxy)-3-methyl-butyl]-thiophene-2-carbonyl}-amino)-propionic acid;
(R,S)-3-({5-[1-(4′-Cyano-2,6-dimethyl-biphenyl-4-yloxy)-3-methyl-butyl]-thiophene-2-carbonyl}-amino)-propionic acid;
(R,S)-3-({5-[1-(2,6-Dimethyl-4′-trifluoromethoxy-biphenyl-4-yloxy)-3-methyl-butyl]-thiophene-2-carbonyl}-amino)-propionic acid;
(R,S)-3-({5-[1-(4′-Dimethylamino-2,6-dimethyl-biphenyl-4-yloxy)-3-methyl-butyl]-thiophene-2-carbonyl}-amino)-propionic acid;
(R,S)-3-[(5-{1-[4-(5-Acetyl-thiophen-2-yl)-3,5-dimethyl-phenoxy]-3-methyl-butyl]-thiophene-2-carbonyl}-amino)-propionic acid;
(R,S)-3-[(5-{1-[4-(5-Cyano-thiophen-2-yl)-3,5-dimethyl-phenoxy]-3-methyl-butyl}-thiophene-2-carbonyl)-amino]-propionic acid;
(R,S)-3-({5-[1-(3,5-Dimethyl-4-thiophen-3-yl-phenoxy)-3-methyl-butyl]-thiophene-2-carbonyl}-amino)-propionic acid;
(R,S)-3-({5-[1-(4′-tert-Butyl-2,6-dimethyl-biphenyl-4-ylsulfanyl)-4,4,4-trifluoro-butyl]-thiophene-2-carbonyl}-amino)-propionic acid;
(R,S)-3-({5-[1-(2,6-Dimethyl-4′-trifluoromethyl-biphenyl-4-ylsulfanyl)-4,4,4-trifluoro-butyl]-thiophene-2-carbonyl}-amino)-propionic acid;
(R,S)-3-({5-[1-(2,6-Dimethyl-4′-trifluoromethyl-biphenyl-4-yloxy)-4,4,4-trifluoro-butyl]-thiophene-2-carbonyl}-amino)-propionic acid;
3-({5-[1-(4′-tert-Butyl-2,6-dimethyl-biphenyl-4-yloxy)-4,4,4-trifluoro-butyl]-thiophene-2-carbonyl}-amino)-propionic acid;
3-({5-[1-(2,6-Dimethyl-4′-trifluoromethyl-biphenyl-4-yloxy)-4,4,4-trifluoro-butyl]-thiophene-2-carbonyl}-amino)-propionic acid;
3-({5-[1-(2,6-Dimethyl-4′-trifluoromethoxy-biphenyl-4-yloxy)-2-methyl-propyl]-thiophene-2-carbonyl}-amino)-propionic acid;
3-({5-[1-(4′-Isopropyl-2,6-dimethyl-biphenyl-4-yloxy)-2-methyl-propyl]-thiophene-2-carbonyl}-amino)-propionic acid;
(R,S)-3 -({5-[1-(4′-tert-Butyl-2,6-dimethyl-biphenyl-4-yloxy)-2-methyl-propyl]-thiophene-2-carbonyl}-amino)-2R-hydroxy-propionic acid;
(R,S)-3-({5-[1-(2,6-Dimethyl-4′-trifluoromethyl-biphenyl-4-yloxy)-2-methyl-propyl]-thiophene-2-carbonyl}-amino)-2S-hydroxy-propionic acid;
(R,S)-3-({5-[1-(4′-tert-Butyl-2,6-dimethyl-biphenyl-4-yloxy)-2-methyl-propyl]-thiophene-2-carbonyl}-amino)-2S-hydroxy-propionic acid;
3-({5-[1-(4′-tert-Butyl-2-methyl-biphenyl-4-yloxy)-butyl]-thiophene-2-carbonyl}-amino)-propionic acid;
3-({5-[1-(4′-tert-Butyl-2,6-dimethyl-biphenyl-4-yloxy)-ethyl]-thiophene-2-carbonyl}-amino)-propionic acid;
3-({5-[1-(4′-tert-Butyl-2,6 -dimethyl-biphenyl-4-yloxy)-butyl]-thiophene-2-carbonyl}-amino)-propionic acid;
3-({5-[1-(2,6-Dimethyl-4′-trifluoromethyl-biphenyl-4-yloxy)-pentyl]-thiophene-2-carbonyl}-amino)-propionic acid;
3-({5-[1-(2,6-Dimethyl-4′-trifluoromethyl-biphenyl-4-yloxy)-2-methyl-propyl]-thiophene-2-carbonyl}-amino)-2R-hydroxy-propionic acid;
3-({5-[1-(2,6-Dimethyl-4′-trifluoromethyl-biphenyl-4-yloxy)-2-methyl-propyl]-thiophene-2-carbonyl}-amino)-2S-hydroxy-propionic acid;
3-({5-[1-(4′-tert-Butyl-2,6 -dimethyl-biphenyl-4-yloxy)-2-methyl-propyl]-thiophene-2-carbonyl}-amino)-2R-hydroxy-propionic acid;
3-({5-[1-(4′-tert-Butyl-2,6 -dimethyl-biphenyl-4-yloxy)-2-methyl-propyl]-thiophene-2-carbonyl}-amino)-2S-hydroxy-propionic acid;
3-({5-[1-(4′-tert-Butyl-2,6 -dimethyl-biphenyl-4-yloxymethyl)-1-ethyl-propyl]-thiophene-2-carbonyl}-amino)-propionic acid;
(R,S)-3-({5-[1-(4′-tert-Butyl-2,6 -dimethyl-biphenyl-4-yloxymethyl)-propyl]-thiophene-2-carbonyl}-amino)-propionic acid;
(R,S)-3-({5-[1-(2,6-Dimethyl-4′-trifluoromethyl-biphenyl-4-yloxymethyl)-propyl]-thiophene-2-carbonyl}-amino)-propionic acid;
3-({5-[1-(4′-tert-Butyl-2,6 -dimethyl-biphenyl-4-yloxymethyl)-propyl]-thiophene-2-carbonyl}-amino)-propionic acid;
3-({5-[1-(2,6-Dimethyl-4′-trifluoromethyl-biphenyl-4-yloxymethyl)-propyl]-thiophene-2-carbonyl}-amino)-propionic acid;
(R,S)-3-({5-[1-(4′-tert-Butyl-2,6 -dimethyl-biphenyl-4-yloxy)-propyl]-4-chloro-thiophene-2-carbonyl}-amino)-propionic acid;
(R,S)-3-({5-[1-(4′-tert-Butyl-2,6-dimethyl-biphenyl-4-ylsulfanyl)-ethyl]-thiophene-2-carbonyl}-amino)-propionic acid;
(R,S)-3-({5-[1-(4′-tert-Butyl-2,6-dimethyl-biphenyl-4-ylsulfanyl)-ethyl]-thiophene-2-carbonyl}-amino)-propionic acid;
(R,S)-3-({5-[1-(2,6-Dimethyl-4′-trifluoromethyl-biphenyl-4-ylsulfanyl)-2,2-dimethyl-propyl]-thiophene-2-carbonyl}-amino)-propionic acid;
(R,S)-3-({5-[1-(2,6-Dimethyl-4′-trifluoromethyl-biphenyl-4-ylsulfanyl)-3-methyl-butyl]-thiophene-2-carbonyl}-amino)-propionic acid;
3-({5-[1-(4′-tert-Butyl-2,6-dimethyl-biphenyl-4-ylsulfanyl)-ethyl]-thiophene-2-carbonyl}-amino)-propionic acid;
3-({5-[1-(2,6-Dimethyl-4′-trifluoromethyl-biphenyl-4-ylsulfanyl)-2,2-dimethyl-propyl]-thiophene-2-carbonyl}-amino)-propionic acid;
(R,S)-3-({5-[1-(2,6-Dimethyl-4′-trifluoromethyl-biphenyl-4-ylsulfanyl)-3,3-dimethyl-butyl]thiophene-2-carbonyl}-amino)-propionic acid;
(R,S)-3-({5-[1-(2,6-Dimethyl-4′-trifluoromethyl-biphenyl-4-ylsulfanyl)-pentyl]-thiophene-2-carbonyl}-amino)-propionic acid;
(R,S)-3-({5-[1-(2,6-Dimethyl-4′-trifluoromethyl-biphenyl-4-ylsulfanyl)-butyl]-thiophene-2-carbonyl}-amino)-propionic acid;
3-({5-[1-(2,6-Dimethyl-4′-trifluoromethyl-biphenyl-4-ylsulfanyl)-butyl]-thiophene-2-carbonyl}-amino)-propionic acid;
3-({5-[1-(2,6-Dimethyl-4′-trifluoromethyl-biphenyl-4-yloxy)-3-methyl-butyl]-thiophene-2-carbonyl}-amino)-propionic acid;
3-({5-[1-(4′-tert-Butyl-2,6 -dimethyl-biphenyl-4-yloxy)-2,2-dimethyl-propyl]-thiophene-2-carbonyl}-amino)-propionic acid;
3-({5-[1-(4′-tert-Butyl-2,6 -dimethyl-biphenyl-4-yloxy)-propyl]-thiophene-2-carbonyl}-amino)-propionic acid;
3-({5-[1-(2,6-Dimethyl-4′-trifluoromethyl-biphenyl-4-yloxy)-butyl]-thiophene-2-carbonyl}-amino)-propionic acid;
3-({5-[1-(4′-Isopropyl-2,6-dimethyl-biphenyl-4-yloxy)-butyl]-thiophene-2-carbonyl}-amino)-propionic acid;
3-({5-[1-(2,6-Dimethyl-4′-trifluoromethoxy-biphenyl-4-yloxy)-butyl]-thiophene-2-carbonyl}-amino)-propionic acid;
3-({5-[1-(4′-tert-Butyl-2,6-dimethyl-biphenyl-4-ylsulfanyl)-2,2-dimethyl-propyl]-thiophene-2-carbonyl}-amino)-propionic acid;
3-({5-[1-(4′-tert-Butyl-2,6-dimethyl-biphenyl-4-ylsulfanyl)-3-methyl-butyl]-thiophene-2-carbonyl}-amino)-propionic acid;
3-({5-[1-(4′-Isopropyl-2,6-dimethyl-biphenyl-4-ylsulfanyl)-2-methyl-propyl]-thiophene-2-carbonyl}-amino)-propionic acid;
3-({5-[1-(4′-tert-Butyl-2,6-dimethyl-biphenyl-4-ylsulfanyl)-propyl]-thiophene-2-carbonyl}-amino)-propionic acid;
(R,S)-3-({5-[1-(4′-tert-Butyl-2,6-dimethyl-biphenyl-4-ylsulfanyl)-2-methyl-propyl]-thiophene-2-carbonyl}-amino)-propionic acid;
(R,S)-3-({5-[1-(4′-tert-Butyl-2,6-dimethyl-biphenyl-4-ylsulfanyl)-2,2-dimethyl-propyl]-thiophene-2-carbonyl}-amino)-propionic acid;
(R,S)-3-({5-[1-(4′-tert-Butyl-2,6-dimethyl-biphenyl-4-ylsulfanyl)-3,3-dimethyl-butyl]-thiophene-2-carbonyl}-amino)-propionic acid;
(R,S)-3-({5-[1-(4′-tert-Butyl-2,6-dimethyl-biphenyl-4-ylsulfanyl)-3-methyl-butyl]-thiophene-2-carbonyl}-amino)-propionic acid;
3-({5-[1-(4′-tert-Butyl-2,6-dimethyl-biphenyl-4-ylsulfanyl)-1-methyl-ethyl]-thiophene-2-carbonyl}-amino)-propionic acid;
(R,S)-3-({5-[1-(2,6-Dimethyl-4′-trifluoromethyl-biphenyl-4-ylsulfanyl)-propyl]-thiophene-2-carbonyl}-amino)-propionic acid;
(R,S)-3-({5-[1-(4′-tert-Butyl-2,6-dimethyl-biphenyl-4-ylsulfanyl)-propyl]-thiophene-2-carbonyl}-amino)-propionic acid;
(R,S)-3-({5-[1-(4′-tert-Butyl-2,6 -dimethyl-biphenyl-4-ylsulfanyl)-4,4-dimethyl-pentyl]thiophene-2-carbonyl}-amino)-propionic acid;
(R,S)-3-({5-[1-(4′-Isopropyl-2,6-dimethyl-biphenyl-4-ylsulfanyl)-2-methyl-propyl]-thiophene-2-carbonyl}-amino)-propionic acid;
(R,S)-3-({5-[1-(4′-tert-Butyl-2,6 -dimethyl-biphenyl-4-ylsulfanyl)-butyl]-thiophene-2-carbonyl}-amino)-propionic acid;
(R,S)-3-({5-[1-(4′-tert-Butyl-2,6 -dimethyl-biphenyl-4-ylsulfanyl)-pentyl]-thiophene-2-carbonyl}-amino)-propionic acid;
3-({5-[4,4,4-Trifluoro-1-(4′-isopropyl-2,6-dimethyl-biphenyl-4-yloxy)-butyl]-thiophene-2-carbonyl}-amino)-propionic acid;
3-({5-[1-(2,6-Dimethyl-4′-trifluoromethoxy-biphenyl-4-yloxy)-4,4,4-trifluoro-butyl]-thiophene-2-carbonyl}-amino)-propionic acid;
3-({5-[1-(4′-Ethyl-2,6-dimethyl-biphenyl-4-yloxy)-4,4,4-trifluoro-butyl]-thiophene-2-carbonyl}-amino)-propionic acid;
3-({5-[1-(4′-tert-Butyl-2,6-dimethyl-biphenyl-4-ylsulfanyl)-4,4,4-trifluoro-butyl]-thiophene-2-carbonyl}-amino)-propionic acid;
3-{4-[1-(4′-tert-butyl-2,6-dimethyl-biphenyl-4-yloxy)-4,4,4-trifluoro-butyl]-benzoylamino}-propionic acid;
3-{4-[1-(4′-Trifluoromethyl-biphenyl-4-yloxy)-propyl]-benzoylamino }-propionic acid;
3-{4-[3-Methyl-1-(4′-trifluoromethyl-biphenyl-4-yloxy)-butyl]-benzoylamino}-propionic acid;
3-{4-[1-(4-tert-Butyl-phenoxy)-3-methyl-butyl]-benzoylamino}-propionic acid;
3-{4-[1-(4′-tert-Butyl-biphenyl-4-yloxy)-4,4,4-trifluoro-butyl]-benzoylamino}-propionic acid;
3-{4-[4,4,4-Trifluoro-1-(4′-trifluoromethyl-biphenyl-4-yloxy)-butyl]-benzoylamino}-propionic acid;
3-{4-[(4-Bromo-phenyl)-(4′-tert-butyl-biphenyl-4-yloxy)-methyl]-benzoylamino}-propionic acid;
3-{4-[2-Methyl-1-(4′-trifluoromethyl-biphenyl-4-yloxy)-propyl]-benzoylamino}-propionic acid;
3-{4-[1-(4′-tert-Butyl-biphenyl-4-yloxy)-propyl]-benzoylamino}-propionic acid;
3-{4-[1-(4′-tert-Butyl-biphenyl-4-yloxy)-heptyl]-benzoylamino}-propionic acid;
3-{4-[1-(4′-tert-Butyl-biphenyl-4-yloxy)-3-methyl-butyl]-benzoylamino}-propionic acid;
3-{4-[1-(4-Cyclohexyl-phenoxy)-hexyl]-benzoylamino}-propionic acid;
3-{4-[1-(4-Benzyloxy-phenoxy)-hexyl]-benzoylamino}-propionic acid;
3-[4-(1-Phenoxy-hexyl)-benzoylamino]-propionic acid;
3-{4-[1-(4′-Trifluoromethyl-biphenyl-4-yloxy)-heptyl]-benzoylamino}-propionic acid;
3-{4-[1-(4′-Trifluoromethyl-biphenyl-4-ylsulfanyl)-heptyl]-benzoylamino}-propionic acid;
3-{4-[1-(4′-Trifluoromethyl-biphenyl-4-yloxy)-pentyl]-benzoylamino}-propionic acid;
3-{4-[1-(4′-tert-Butyl-biphenyl-4-yloxy)-pentyl]-benzoylamino}-propionic acid;
3-{4-[1-(4′-Trifluoromethyl-biphenyl-4-yloxy)-butyl]-benzoylamino}-propionic acid;
3-{4-[1-(4′-tert-Butyl-biphenyl-4-yloxy)-butyl]-benzoylamino}-propionic acid;
3-{4-[3-Methyl-1-(4′-trifluoromethyl-biphenyl-4-ylsulfanyl)-butyl]-benzoylamino}-propionic acid;
3-{4-[1-(4′-Trifluoromethyl-biphenyl-4-yloxy)-nonyl]-benzoylamino}-propionic acid;
3-(4-{3-Methyl-1-[4-(6-trifluoromethyl-pyridin-3-yl)-phenoxy]-butyl}-benzoylamino)-propionic acid;
3-(4-{2-Methyl-1-[4-(6-trifluoromethyl-pyridin-3-yl)-phenoxy]-propyl}-benzoylamino)-propionic acid;
3-(4-{1-[4′-(1-trifluoromethoxy-biphenyl-4-ylsulfanyl]-3-methyl-butyl}-benzoylamino)-propionic acid;
3-(4-{1-[4′-(1-Fluoro-ethoxy)-biphenyl-4-ylsulfanyl]-3-methyl-butyl}-benzoylamino)-propionic acid;
3-{4-[1-(3′,4′-dimethyl-biphenyl-4-ylsulfanyl)-3-methyl-butyl]-benzoylamino}-propionic acid;
3-{4-[1-(4′-cyano-biphenyl-4-ylsulfanyl)-3-methyl-butyl]-benzoylamino}-propionic acid;
3-{4-[1-(4′-Isobutyl-biphenyl-4-ylsulfanyl)-3-methyl-butyl]-benzoylamino}-propionic acid;
3-(4-{1-[4-(6-Methoxy-pyridin-3-yl)-phenylsulfanyl]-3-methyl-butyl}-benzoylamino)-propionic acid;
3-{4-[1-(4′-Ethyl-biphenyl-4-yloxy)-heptyl]-benzoylamino}-propionic acid;
3-{4-[1-(4-Benzyloxy-phenoxy)-hexyl]-benzoylamino}-propionic acid;
3-{4-[3-Methyl-1-(4′-trifluoromethyl-biphenyl-4-yloxy)-butyl]-benzoylamino}-propionic acid;
3-{4-[1-(4′-Trifluoromethyl-biphenyl-4-yloxy)-propyl]-benzoylamino}-propionic acid;
3-{4-[1-(4′-tert-Butyl-biphenyl-4-yloxy)-propyl]-benzoylamino}-propionic, acid;
3-{4-[1-(4′-tert-Butyl-biphenyl-4-yloxy)-heptyl]-benzoylamino}-propionic acid;
3-{4-[1-(4′-tert-Butyl-biphenyl-4-yloxy)-3-methyl-butyl]-benzoylamino}-propionic acid;
3-{4-[1-(4′-Trifluoromethyl-biphenyl-4-ylsulfanyl)-heptyl]-benzoylamino}-propionic acid;
3-{4-[1-(4′-tert-Butyl-biphenyl-4-yloxy)-pentyl]-benzoylamino}-propionic acid;
3-{4-[2-Methyl-1-(4′-trifluoromethyl-biphenyl-4-yloxy)-propyl]-benzoylamino}-propionic acid;
3-{4-[1-(4′-Trifluoromethyl-biphenyl-4-yloxy)-pentyl]-benzoylamino}-propionic acid;
3-{4-[1-(4′-Trifluoromethyl-biphenyl-4-yloxy)-butyl]-benzoylamino}-propionic acid;
3-{4-[1-(4′-tert-Butyl-biphenyl-4-yloxy)-butyl]-benzoylamino}-propionic acid;
3-(4-{1-[4′-(1-Fluoro-1-methyl-ethyl)-biphenyl-4-ylsulfanyl]-3-methyl-butyl}-benzoylamino)-propionic acid;
3-(4-{3-Methyl-1-1-[4-(6-trifluoromethyl-pyridin-3 -yl)-phenoxy]-butyl}-benzoylamino)-propionic acid;
3-{4-[1-(4′-Trifluoromethyl-biphenyl-4-yloxy)-nonyl]-benzoylamino}-propionic acid;
3-{4-[1-(4′-Ethyl-biphenyl-4-yloxy)-heptyl]-benzoylamino}-propionic acid;
3-(4-{1-[6-(4-tert-Butyl-phenyl)-pyridin-3-yloxy]-4,4-dimethyl-pentyl}-benzoylamino)-propionic acid;
3-(4-{1-[6-(4-Trifluoromethyl-phenyl)-pyridin-3-yloxy]-heptyl}-benzoylamino)-propionic acid;
3-(4-{4,4,4-Trifluoro-1-[6-(4-trifluoromethyl-phenyl)-pyridin-3-yloxy]-butyl}-benzoylamino)-propionic acid;
3-(4-{3-Methyl-1-[6-(4-trifluoromethyl-phenyl)-pyridin-3-yloxy]-butyl}-benzoylamino)-propionic acid;
3-(4-{4,4-Dimethyl-1-[6-(4-trifluoromethyl-phenyl)-pyridin-3-yloxy]-pentyl}-benzoylamino)-propionic acid;
3-(4-{1-[6-(4-Trifluoromethyl-phenyl)-pyridin-3-yl]-butoxy}-benzoylamino)-propionic acid;
3-(4-{1-[6-(4-tert-Butyl-phenyl)-pyridin-3 -yloxy]-4,4,4-trifluoro-butyl}-benzoylamino)-propionic acid;
3-(4-{1-[6-(4-tert-Butyl-phenyl)-pyridin-3-yloxy]-3-methyl-butyl}-benzoylamino)-propionic acid;
3-(4-{1-[6-(4-tert-Butyl-phenyl)-pyridin-3-yloxy]-4,4-dimethyl-pentyl}-benzoylamino)-propionic acid;
3-(4-{1-[6-(4-Trifluoromethyl-phenyl)-pyridin-3-yloxy]-heptyl}-benzoylamino)-propionic acid;
3-(4-{4,4,4-Trifluoro-1-[6-(4-trifluoromethyl-phenyl)-pyridin-3-yloxy]-butyl}-benzoylamino)-propionic acid;
3-(4-{1-[6-(4-tert-Butyl-phenyl)-pyridin-3-yloxy]-butyl}-benzoylamino)-propionic acid;
3-(4-{1-[6-(4-Isobutyl-phenyl)-pyridin-3-yloxy]-butyl}-benzoylamino)-propionic acid;
3-{4-[1-(3′-Trifluoromethyl-biphenyl-4-ylsulfanyl)-heptyl]-benzoylamino}-propionic acid;
3-{4-[1-(4′-Acetyl-biphenyl-4-ylsulfanyl)-heptyl]-benzoylamino}-propionic acid;
3-{4-[1-(3′,4′-dimethyl-biphenyl-4-ylsulfanyl)-heptyl]-benzoylamino}-propionic acid;
3-{4-[1-(4′-methylsulfonyl-biphenyl-4-ylsulfanyl)-heptyl]-benzoylamino}-propionic acid;
3-{4-[1-(2′,3′-dimethyl-biphenyl-4-ylsulfanyl)-heptyl]-benzoylamino}-propionic acid;
3-{4-[1-(2′,6′-dimethyl-biphenyl-4-ylsulfanyl)-heptyl]-benzoylamino}-propionic acid;
3-{4-[1-(3′-isopropyl-biphenyl-4-ylsulfanyl)-heptyl]-benzoylamino}-propionic acid;
3-{4-[1-(3′-acetyl-biphenyl-4-ylsulfanyl)-heptyl]-benzoylamino}-propionic acid;
3-{4-[1-(4′-pentyl-biphenyl-4-ylsulfanyl)-heptyl]-benzoylamino}-propionic acid;
3-{4-[1-(4′-cyclohexyl-biphenyl-4-ylsulfanyl)-heptyl]-benzoylamino}-propionic acid;
3-{4-[1-(4-Allyloxy-phenoxy)-heptyl]-benzoylamino}-propionic acid;
3-{4-[1-(2,2,3,3-Tetrafluoro-2,3-dihydro-benzo[1,4]dioxin-6-yloxy)-heptyl]-benzoylamino}-propionic acid;
3-(4-{1-[4-(4-Trifluoromethyl-phenoxy)-phenoxy]-heptyl}-benzoylamino)-propionic acid;
3-{4-[1-(4-Pentyl-phenoxy)-heptyl]-benzoylamino}-propionic acid;
3-(4-{1-[4-(4-tert-Butyl-benzyloxy)-phenoxy]-heptyl}-benzoylamino)-propionic acid;
3-(4-{1-[4-(3,5-bistrifluoromethyl-benzyloxy)-phenoxy]-heptyl}-benzoylamino)-propionic acid;
3-(4-{1-[4-(4-isopropyl-benzyloxy)-phenoxy]-heptyl}-benzoylamino)-propionic acid;
3-(4-{1-[4-(4-chloro-benzyloxy)-phenoxy]-heptyl}-benzoylamino)-propionic acid;
3-(4-{1-[4-(4-ethyl-benzyloxy)-phenoxy]-heptyl}-benzoylamino)-propionic acid;
3-(4-{1-[4-(4-bromo-benzyloxy)-phenoxy]-heptyl}-benzoylamino)-propionic acid;
3-(4-{1-[4-(4-fluoro-benzyloxy)-phenoxy]-heptyl}-benzoylamino)-propionic acid;
3-(4-{1-[4-(4-trifluoromethyl-benzyloxy)-phenoxy]-heptyl}-benzoylamino)-propionic acid;
3-(4-{1-[4-(4-phenyl-benzyloxy)-phenoxy]-heptyl}-benzoylamino)-propionic acid;
3-(4-{1-[4-(3-chloro-benzyloxy)-phenoxy]-heptyl}-benzoylamino)-propionic acid;
3-(4-{1-[4(3,4-dimethyl-benzyloxy)-phenoxy]-heptyl}-benzoylamino)-propionic acid;
3-(4-{1-[4-(4-isopropoxyphenoxy]-heptyl}-benzoylamino)-propionic acid;
3-{4-[1-(3′,5′-bistrifluoromethyl-biphenyl-4-yloxy)-heptyl]-benzoylamino}-propionic acid;
3-{4-[1-(4′-tert-Butyl-biphenyl-4-yloxy)-4,4-dimethyl-pentyl]-benzoylamino}-propionic acid;
3-{4-[1-(4′-trifluoromethyl-biphenyl-4-yloxy)-4,4-dimethyl-pentyl]-benzoylamino}-propionic acid;
3-{4-[1-(4′-tert-Butyl-biphenyl-4-yloxy)-2-methyl-propyl]-benzoylamino}-propionic acid;
3-{4-[1-(4′-trifluoromethyl-biphenyl-4-yloxy)-hexyl]-benzoylamino}-propionic acid;
3-{4-[1-(3′,5′-bistrifluoromethyl-biphenyl-4-yloxy)-heptyl]-benzoylamino}-propionic acid;
3-{4-[1(4-tert-Butyl-phenoxy)-3-methyl-butyl]-benzoylamino}-propionic acid;
3-{4-[1-(4′-tertbutyl-biphenyl-4-yloxy)-2-methyl-propyl]-benzoylamino}-propionic acid;
3-{4-[1-(4′-trifluoromethyl-biphenyl-4-yloxy)-4,4-dimethyl-pentyl]-benzoylamino}-propionic acid;
3-{4-[1-(4′-Trifluoromethoxy-biphenyl-4-yloxy)-heptyl]-benzoylamino}-propionic acid;
3-{4-[1-(4′-Trifluoromethyl-biphenyl-4-yloxy)-heptyl]-benzoylamino}-propionic acid;
3-{4-[1-(2,2,3,3 -Tetrafluoro-2,3 -dihydro-benzo[1,4]dioxin-6-yloxy)-heptyl]-benzoylamino}-propionic acid;
3-(4-{1-[4-(4-Trifluoromethyl-phenoxy)-phenoxy]-heptyl}-benzoylamino)-propionic acid;
3-{4-[α-(4′-Trifluoromethyl-biphenyl-4-yloxy)-hexyl]-benzoylamino}-propionic acid;
3-(4-{1-[4′-isopropyl-biphenyl-4-ylsulfanyl]-butyl}-benzoylamino)-propionic acid;
3-{4-[1-(2,6-Dimethyl-4′-trifluoromethoxy-biphenyl-4-yloxy)-heptyl]-benzoylamino}-propionic acid;
3-{4-[1-(2,6-Dimethyl-4′-trifluoromethyl-biphenyl-4-yloxy)-heptyl]-benzoylamino}-propionic acid;
3-{4-[1-(2,6-Dimethyl-4′-tertbutyl-biphenyl-4-yloxy)-heptyl]-benzoylamino}-propionic acid;
3-{4-[1-(4′-tert-Butyl-biphenyl-4-yloxy)-3-methyl-butyl]-benzoylamino}-2(R)-hydroxy-propionic acid;
3-{4-[1-(4′-tert-Butyl-biphenyl-4-yloxy)-3-methyl-butyl]-benzoylamino}-2(S)-hydroxy-propionic acid;
3-{4-[1-(4′-Pentyl-biphenyl-4-yloxy)-butyl]-benzoylamino}-propionic acid;
3-{4-[1-(4′-Isobutyl-biphenyl-4-yloxy)-butyl]-benzoylamino}-propionic acid;
3-{4-[1-(6-chloro-pyridin-3-yloxy)-4,4,4-trifluoro-butyl]-benzoylamino}-propionic acid;
3-{4-[1-(4′-Acetyl-biphenyl-4-yloxy)-butyl]-benzoylamino}-propionic acid;
3-{4-[1-(3′,5′-dichloro-biphenyl-4-yloxy)-butyl]-benzoylamino}-propionic acid;
3-{4-[1-(2′,3′,4′-trifluoro-biphenyl-4-yloxy)-butyl]-benzoylamino}-propionic acid;
3-{4-[1-(2′,4′-dimethoxy-biphenyl-4-yloxy)-butyl]-benzoylamino}-propionic acid;
3-{4-[1-(4′-t-butyl-biphenyl-4-yloxy)-hexyl]-benzoylamino}-propionic acid;
3-{4-[1-(4′-pentylphenyl-4-yloxy)-hexyl]-benzoylamino}-propionic acid;
3-(4-{1-[1-(1-Methyl-1-phenyl-ethyl)-phenoxy]-heptyl}-benzoylamino)-propionic acid;
3-{4-[1-(2′,4′,6′-trimethyl-biphenyl-4-yloxy)-heptyl]-benzoylamino}-propionic acid;
3-{4-[1-(4′-Fluoro-2′-methyl-biphenyl-4-yloxy)-heptyl]-benzoylamino}-propionic acid;
3-{4-[1-(4′-Trifluoromethoxy-biphenyl-4-yloxy)-hexyl]-benzoylamino}-propionic acid;
3-{4-[1-(4′-Fluoro-biphenyl-4-yloxy)-heptyl]-benzoylamino}-propionic acid;
3-{4-[Cyclopropyl-(4′-trifluoromethyl-biphenyl-4-yloxy)-methyl]-benzoylamino}-propionic acid;
3-{4-[1-(4′-tert-Butyl-biphenyl-4-yloxy)-4,4,4-trifluoro-butyl]-benzoylamino}-propionic acid;
3-{4-[1-(4′-Chloro-biphenyl-4-yloxy)-heptyl]-benzoylamino}-propionic acid;
3-{4-[1-(4′-tert-Butyl-2-methyl-biphenyl-4-yloxy)-3-methyl-butyl]-3-fluoro-benzoylamino}-propionic acid;
3-{4-[1-(4′-tert-Butyl-biphenyl-4-yloxy)-heptyl]-3-fluoro-benzoylamino}-propionic acid;
3-{3-Fluoro-4-[1-(2-methyl-4′-trifluoromethyl-biphenyl-4-yloxy)-heptyl]-benzoylamino}-propionic acid;
3-{4-[1-(4′-tert-Butyl-biphenyl-4-yloxy)-3-methyl-butyl]-3-fluoro-benzoylamino}-propionic acid;
3-{4-[1-(4′-tert-Butyl-2,6-dimethyl-biphenyl-4-yloxy)-heptyl]-benzoylamino}-propionic acid;
3-{4-[1-(4′-tert-Butyl-2-methyl-biphenyl-4-yloxy)-heptyl]-benzoylamino}-propionic acid;
3-{4-[1-(2-Methyl-4′-trifluoromethyl-biphenyl-4-yloxy)-heptyl]-benzoylamino}-propionic acid;
3-{4-[1-(4′-tert-Butyl-2,6-dimethyl-biphenyl-4-yloxy)-3-methyl-butyl]-benzoylamino}-propionic acid;
3-{4-[1-(4′-Isopropyl-2-methyl-biphenyl-4-yloxy)-heptyl]-benzoylamino}-propionic acid;
3-{4-[1-(4′-Fluoro-2-methyl-biphenyl-4-yloxy)-heptyl]-benzoylamino}-propionic acid;
3-{4-[1-(4′-Ethyl-2-methyl-biphenyl-4-yloxy)-heptyl]-benzoylamino}-propionic acid;
3-{4-[1-(2,6-Dimethyl-biphenyl-4-yloxy)-3-methyl-butyl]-benzoylamino}-propionic acid;
3-{4-[1-(4-Ethyl-2,6-dimethyl-biphenyl-4-yloxy)-3-methyl-butyl]-benzoylamino}-propionic acid;
3-{4-[1-(4′-tert-Butyl-2-trifluoromethyl-biphenyl-4-yloxy)-3-methyl-butyl]-benzoylamino}-propionic acid;
3-{4-[1-(4′-tert-Butyl-2-trifluoromethyl-biphenyl-4-yloxy)-heptyl]-benzoylamino}-propionic acid;
3-{4-[1-(4′-tert-Butyl-2-trifluoromethyl-biphenyl-4-yloxy)-pentyl]-benzoylamino}-propionic acid;
3-{4-[1-(4′-tert-Butyl-2-trifluoromethyl-biphenyl-4-yloxy)-2-methyl-propyl]-benzoylamino}-propionic acid;
3-{4-[1-(2,6-Dimethyl-4′-trifluoromethyl-biphenyl-4-yloxy)-4,4-dimethyl-pentyl]-benzoylamino}-propionic acid;
3-{4-[1-(4′-Isopropyl-2,6-dimethyl-biphenyl-4-yloxy)-4,4-dimethyl-pentyl]-benzoylamino}-propionic acid, 3-{4-[1-(2-Cyano-4′-isopropyl-biphenyl-4-yloxy)-3-methyl-butyl]-benzoylamino}-propionic acid;
3-{4-[1-(4′-Isopropyl-2,6-dimethyl-biphenyl-4-yloxy)-2-methyl-propyl]-benzoylamino}-propionic acid;
3-{4-[1-(2-Ethyl-4′-trifluoromethyl-biphenyl-4-yloxy)-heptyl]-benzoylamino}-propionic acid;
3-{4-[1-(4′-Chloro-2,6-dimethyl-biphenyl-4-yloxy)-4,4-dimethyl-pentyl]-benzoylamino}-propionic acid;
3-{4-[1-(4′-tert-Butyl-2,6-dimethyl-biphenyl-4-yloxy)-2-methyl-propyl]-benzoylamino}-propionic acid;
3-{4-[2-Methyl-1-(2-methyl-4′-trifluoromethoxy-biphenyl-4-yloxy)-propyl]-benzoylamino}-propionic acid;
3-{4-[1-(2-Chloro-4′-isopropyl-biphenyl-4-yloxy)-3-methyl-butyl]-benzoylamino}-propionic acid;
3-{4-[1-(2-Chloro-4′-trifluoromethyl-biphenyl-4-yloxy)-3-methyl-butyl]-benzoylamino}-propionic acid;
3-{4-[2-Methyl-1-(2-methyl-4′-trifluoromethyl-biphenyl-4-yloxy)-propyl]-benzoylamino}-propionic acid;
3(4-{3-Methyl-1-[5-methyl-6-(4-trifluoromethyl-phenyl)-pyridin-3 -yloxy]-butyl}-benzoylamino)-propionic acid;
3-(4-{1-[6-(4-Isopropyl-phenyl)-5-methyl-pyridin-3 -yloxy]-3-methyl-butyl}-benzoylamino)-propionic acid;
3-(3-Fluoro-4-{3-methyl-1-[6-(4-trifluoromethyl-phenyl)-pyridin-3-yloxy]-butyl}-benzoylamino)-propionic acid;
3-(4-{3-Methyl-1-1-[4-(5-trifluoromethyl-pyridin-2-yl)-phenoxy]-butyl}-benzoylamino)-propionic acid;
3-{4-[1-(4-tert-Butyl-phenoxy)-3-methyl-butyl]-benzoylamino}-propionic acid;
2-Hydroxy-3-{4-[1-(4′-trifluoromethyl-biphenyl-4-yloxy)-heptyl]-benzoylamino}-propionic acid;
2-Hydroxy-3-{4-[1-(4′-trifluoromethyl-biphenyl-4-yloxy)-heptyl]-benzoylamino}-propionic acid;
3-(4-{1-[4-(1,1,3,3-Tetramethyl-butyl)-phenoxy]-heptyl}-benzoylamino)-propionic acid;
3-{4-[1-(2,2,3,3-Tetrafluoro-2,3-dihydro-benzo[1,4]dioxin-6-yloxy)-heptyl]-benzoylamino}-propionic acid;
3-{4-[1-(2,6-Dimethyl-4′-trifluoromethyl-biphenyl-4-yloxy)-3,3-dimethyl-butyl]-benzoylamino}-propionic acid;
3-{4-[1-(4′-tert-Butyl-2,6-dimethyl-biphenyl-4-yloxy)-3,3-dimethyl-butyl]-benzoylamino}-propionic acid;
3-{4-[1-(4′-Isopropyl-2,6-dimethyl-biphenyl-4-yloxy)-3,3-dimethyl-butyl]-benzoylamino}-propionic acid;
3-(4-{3,3-Dimethyl-1-[5-methyl-6-(4-trifluoromethyl-phenyl)-pyridin-3-yloxy]-butyl}-benzoylamino)-propionic acid;
3-{4-[1-(4′-Isopropyl-2-methoxy-biphenyl-4-yloxy)-3-methyl-butyl]-benzoylamino}-propionic acid;
3-{4-[1-(4′-Isopropyl-2-methyl-biphenyl-4-yloxy)-3-methyl-butyl]-benzoylamino}-propionic acid;
3-{4-[1-(2,6-Dimethyl-4′-trifluoromethoxy-biphenyl-4-yloxy)-3,3-dimethyl-butyl]-benzoylamino}-propionic acid;
3-(4-{1-[2-(tert-Butoxyimino-methyl)-4′-trifluoromethyl-biphenyl-4-yloxy]-3-methyl-butyl}-benzoylamino)-propionic acid;
3-{4-[1-(4′-tert-Butyl-2,6-dimethyl-biphenyl-4-yloxy)-3,3-dimethyl-butyl]-benzoylamino}-2-hydroxy-propionic acid;
3-{4-[1-(4′-tert-Butyl-2,6-dimethyl-biphenyl-4-yloxy)-3,3-dimethyl-butyl]-benzoylamino}-2-hydroxy-propionic acid;
3-{4-[1-(4′-Fluoro-2,6-dimethyl-biphenyl-4-yloxy)-3-methyl-butyl]-benzoylamino}-propionic acid;
3-{4-[1-(4′-Fluoro-2,6,2′-trimethyl-biphenyl-4-yloxy)-heptyl]-benzoylamino}-propionic acid;
3-{4-[1-(4′-Chloro-2,6-dimethyl-biphenyl-4-yloxy)-3-methyl-butyl]-benzoylamino}-propionic acid;
3-{4-[1-(4′-Isopropyl-2,6-dimethyl-biphenyl-4-yloxy)-3-methyl-butyl]-benzoylamino}-propionic acid;
3-{4-[1-(2,6-Dimethyl-4′-trifluoromethyl-biphenyl-4-yloxy)-3-methyl-butyl]-benzoylamino}-propionic acid;
3-{4-[1-(4′-tert-Butyl-biphenyl-4-yloxy)-hexyl]-benzoylamino}-propionic acid;
3-{4-[1-(4′-tert-Butyl-biphenyl-4-yloxy)-heptyl]-3-fluoro-benzoylamino}-propionic acid;
3-{4-[1-(4′-tert-Butyl-biphenyl-4-yloxy)-3-methyl-butyl]-3-fluoro-benzoylamino}-propionic acid;
3-{4-[1-(2-Cyano-4′-isopropyl-biphenyl-4-yloxy)-3-methyl-butyl]-benzoylamino}-propionic acid;
3-{4-[1-(4′-Isopropyl-2-trifluoromethyl-biphenyl-4-yloxy)-heptyl]-benzoylamino}-propionic acid;
3-{4-[1-(2-Ethyl-4′-trifluoromethyl-biphenyl-4-yloxy)-heptyl]-benzoylamino}-propionic acid;
3-[4-(4′-tert-Butyl-2,6-dimethyl-biphenyl-4-yloxymethyl)-benzoylamino]-propionic acid;
3-[4-(4′-tert-Butyl-2,6-dimethyl-biphenyl-4-yloxymethyl)-benzoylamino]-propionic acid;
3-{4-[1-(2,6-Dimethyl-4′-trifluoromethyl-biphenyl-4-yloxy)-ethyl]-benzoylamino}-propionic acid;
3-{4-[1-(4′-tert-Butyl-2,6-dimethyl-biphenyl-4-yloxy)-ethyl]-benzoylamino}-propionic acid;
3-(3-Fluoro-4-{3-methyl-1-[6-(4-trifluoromethyl-phenyl)-pyridin-3-yloxy]-butyl}-benzoylamino)-propionic acid;
3-(4-{1-[4-(1,1,3,3-Tetramethyl-butyl)-phenoxy]-heptyl}-benzoylamino)-propionic acid;
3-(4-{3-Methyl-1-[4-(5-trifluoromethyl-pyridin-2-yl)-phenoxy]-butyl}-benzoylamino)-propionic acid;
3-{4-[1-(4′-tert-Butyl-2,6-dimethyl-biphenyl-4-yloxy)-3,3-dimethyl-butyl]-benzoylamino}-propionic acid;
3-{4-[1-(4′-tert-Butyl-2,6-dimethyl-biphenyl-4-yloxy)-3,3-dimethyl-butyl]-benzoylamino}-2-hydroxy-propionic acid;
3-{4-[1-(4′-tert-Butyl-2,6-dimethyl-biphenyl-4-yloxy)-3,3-dimethyl-butyl]-benzoylamino}-2-hydroxy-propionic acid;
3-{4-[1-(2,6-Dimethyl-4′-trifluoromethyl-biphenyl-4-yloxy)-3-methyl-butyl]-benzoylamino}-2-hydroxy-propionic acid;
3- {4-[1-(2,6-Dimethyl-4′-trifluoromethyl-biphenyl-4-yloxy)-3-methyl-butyl]-benzoylamino}-hydroxy-propionic acid;
3-{4-[1-(4-Bromo-3-[1,3]dioxan-2-yl-phenoxy)-3-methyl-butyl]-benzoylamino}-propionic acid;
3-{4-[1-(4-tert-Butyl-phenoxy)-3-methyl-butyl]-benzoylamino}-propionic acid;
3-{4-[1-(4′-tert-Butyl-biphenyl-4-yloxy)-3-methyl-butyl]-benzoylamino}-2-hydroxy-propionic acid;
3-{4-[1-(4′-tert-Butyl-biphenyl-4-yloxy)-3-methyl-butyl]-benzoylamino}-2-hydroxy-propionic acid;
3-{4-[1-(4-Pentyl-phenoxy)-heptyl]-benzoylamino}-propionic acid;
3-(4-{1-[4-(1-Methyl-1-phenyl-ethyl)-phenoxy]-heptyl}-benzoylamino)-propionic acid;
2-Hydroxy-3-{4-[1-(4′-trifluoromethyl-biphenyl-4-yloxy)-heptyl]-benzoylamino}-propionic acid;
2-Hydroxy-3-{4-[1-(4′-trifluoromethyl-biphenyl-4-yloxy)-heptyl]-benzoylamino}-propionic acid;
3-{4-[1-(4′-Isopropyl-2-methyl-biphenyl-4-yloxy)-3-methyl-butyl]-benzoylamino}-propionic acid;
3-{4-[1-(4-Chloro-3-trifluoromethyl-phenoxy)-heptyl]-benzoylamino}-2-hydroxy-propionic acid;
3-{4-[1-(3-Chloro-4-methyl-phenoxy)-3-methyl-butyl]-benzoylamino}-propionic acid;
3-{4-[1-(2,2,3,3-Tetrafluoro-2,3-dihydro-benzo[1,4]dioxin-6-yloxy)-heptyl]-benzoylamino}-propionic acid;
3-{4-[Cyclopropyl-(4′-trifluoromethyl-biphenyl-4-yloxy)-methyl]-benzoylamino}-propionic acid;
3-{4-[1-(4′-Isopropyl-2,6-dimethyl-biphenyl-4-yloxy)-heptyl]-benzoylamino}-propionic acid;
3-{4-[1-(4′-Acetyl-2,6-dimethyl-biphenyl-4-yloxy)-heptyl]-benzoylamino}-propionic acid;
3-{4-[1-(4′-tert-Butyl-2-methyl-biphenyl-4-yloxy)-heptyl]-benzoylamino}-propionic acid;
3-{4-[1-(2-Methyl-4′-trifluoromethyl-biphenyl-4-yloxy)-heptyl]-benzoylamino}-propionic acid;
3-(4-{1-[6-(4-Isopropyl-phenyl)-5-methyl-pyridin-3-yloxy]-4,4-dimethyl-pentyl}-benzoylamino)-propionic acid;
3-(4-{4,4-Dimethyl-1-[5-methyl-6-(4-trifluoromethyl-phenyl)-pyridin-3-yloxy]-pentyl}-benzoylamino)-propionic acid;
3-(4-{4,4-Dimethyl-1-[5-methyl-6-(4-trifluoromethoxy-phenyl)-pyridin-3-yloxy]-pentyl}-benzoylamino)-propionic acid;
3-{4-[1-(2,6-Dimethyl-4′-trifluoromethoxy-biphenyl-4-yloxy)-3,3-dimethyl-butyl]-benzoylamino}-propionic acid;
3-{4-[1-(2,6-Dimethyl-4′-trifluoromethyl-biphenyl-4-yloxy)-3,3-dimethyl-butyl]-benzoylamino}-2-hydroxy-propionic acid;
3-{4-[1-(2,6-Dimethyl-4′-trifluoromethyl-biphenyl-4-yloxy)-3,3-dimethyl-butyl]-benzoylamino}-2-hydroxy-propionic acid;
2-Hydroxy-3-{4-[1-(4′-isopropyl-2,6-dimethyl-biphenyl-4-yloxy)-3,3-dimethyl-butyl]-benzoylamino}-propionic acid;
3-{4-[1-(4′-Isopropyl-2-methoxy-biphenyl-4-yloxy)-3-methyl-butyl]-benzoylamino}-propionic acid;
3-{3-Fluoro-4-[1-(2-methyl-4′-trifluoromethyl-biphenyl-4-yloxy)-heptyl]-benzoylamino}-propionic acid;
3-{4-[1-(4′-tert-Butyl-2-chloro-biphenyl-4-yloxy)-heptyl]-benzoylamino}-propionic acid;
3-{4-[1-(4′-trifluoromethyl-2-chloro-biphenyl-4-yloxy)-heptyl]-benzoylamino}-propionic acid;
3-{4-[1-(2′,4′-bistrifluoromethyl-2-chloro-biphenyl-4-yloxy)-heptyl]-benzoylamino}-propionic acid;
3-{4-[1-(2,6-Dimethyl-4′-trifluoromethyl-biphenyl-4-yloxy)-3-methyl-butyl]-benzoylamino}-propionic acid;
3-{4-[1-(4′-tert-Butyl-2,6-dimethyl-biphenyl-4-yloxy)-3-methyl-butyl]-benzoylamino}-propionic acid;
3-{4-[1-(2-Hydroxy-4′-isopropyl-biphenyl-4-yloxy)-3-methyl-butyl]-benzoylamino}-propionic acid;
3-{4-[1-(2-[1,3]Dioxan-2-yl-4′-isopropyl-biphenyl-4-yloxy)-3-methyl-butyl]-benzoylamino}-propionic acid;
3-(4-{1-[2-(tert-Butoxyimino-methyl)-4′-isopropyl-biphenyl-4-yloxy]-3-methyl-butyl}-benzoylamino)-propionic acid;
3-(4-{[1[2-(tert-Butoxyimino-methyl)-4′-trifluoromethyl-biphenyl-4-yloxy]-3-methyl-butyl]-benzoylamino}-propionic acid;
3-{4-[1-(2,6-Dimethyl-biphenyl-4-yloxy)-3-methyl-butyl]-benzoylamino}-propionic acid;
3-{4-[1-(4′-Ethyl-2,6-dimethyl-biphenyl-4-yloxy)-3 -methyl-butyl]-benzoylamino}-propionic acid;
3-{4-[1-(2-Methyl-4′-trifluoromethoxy-biphenyl-4-yloxy)-heptyl]-benzoylamino}-propionic acid;
3-{4-[1-(4′-Isopropyl-2-methyl-biphenyl-4-yloxy)-heptyl]-benzoylamino}-propionic acid;
3-{4-[1-(2,6-Dimethyl-4′-trifluoromethoxy-biphenyl-4-yloxy)-3-methyl-butyl]-benzoylamino}-propionic acid;
3-{4-[1-(4′-ethyl-2-methyl-biphenyl-4-yloxy)-heptyl]-benzoylamino}-propionic acid;
3-{4-[1-(4′-acetyl-2-methyl-biphenyl-4-yloxy)-heptyl]-benzoylamino}-propionic acid;
3-{4-[1-(4′-fluoro-2-methyl-biphenyl-4-yloxy)-heptyl]-benzoylamino}-propionic acid;
3-{4-[1-(4′-tert-Butyl-2-trifluoromethyl-biphenyl-4-yloxy)-heptyl]-benzoylamino}-propionic acid;
3-{4-[1-(4′-tert-Butyl-2-trifluoromethyl-biphenyl-4-yloxy)-pentyl]-benzoylamino}-propionic acid;
3-{4-[1-(4′-tert-Butyl-2-trifluoromethyl-biphenyl-4-yloxy)-2-methyl-propyl]-benzoylamino}-propionic acid;
3-{4-[1-(4′-tert-Butyl-2-trifluoromethyl-biphenyl-4-yloxy)-3-methyl-butyl]-benzoylamino}-propionic acid;
3-{4-[1-(3,5 -Dimethyl-4′-trifluoromethyl-biphenyl-4-yloxy)-heptyl]-benzoylamino}-propionic acid;
3-{4-[1-(4′-Chloro-3,5 -dimethyl-biphenyl-4-yloxy)-heptyl]-benzoylamino}-propionic acid;
3-{4-[1-(4′-Chloro-3-methyl-biphenyl-4-yloxy)-heptyl]-benzoylamino}-propionic acid;
3-{4-[1-(2,6-Dimethyl-4′-trifluoromethyl-biphenyl-4-yloxy)-3,3-dimethyl-butyl]-benzoylamino}-propionic acid;
3-{4-[1-(2,6-Dimethyl-4′-trifluoromethyl-biphenyl-4-yloxy)-4,4-dimethyl-pentyl]-benzoylamino}-propionic acid;
3-{4[1-(2,6-Dimethyl-4′-isopropyl-biphenyl-4-yloxy)-4,4-dimethyl-pentyl]-benzoylamino}-propionic acid;
3-{4-[1-(4′-Isopropyl-2,6-dimethyl-biphenyl-4-yloxy)-2-methyl-propyl]-benzoylamino}-propionic acid;
3-{4-[1-(2,6-Dimethyl-4′-trifluoromethyl-biphenyl-4-yloxy)-2-methyl-propyl]-benzoylamino}-propionic acid;
3-{4-[2-Methyl-1-(2-methyl-4′-trifluoromethyl-biphenyl-4-yloxy)-propyl]-benzoylamino}-propionic acid;
3-{4-[1-(2,6-Dimethyl-4′-chloro-biphenyl-4-yloxy)-4,4-dimethyl-pentyl]-benzoylamino}-propionic acid;
3-{4-[1-(4′-tert-Butyl-2,6-dimethyl-biphenyl-4-yloxy)-2-methyl-propyl]-benzoylamino}-propionic acid;
3-{4-[1-(4′-Isopropyl-2-methyl-biphenyl-4-yloxy)-2-methyl-propyl]-benzoylamino}-propionic acid;
3-{4-[1-(2,6-Difluoro-4′-trifluoromethyl-biphenyl-4-yloxy)-3-methyl-butyl]-benzoylamino}-propionic acid;
3-{4-[1-(2,6-Difluoro-4′-isopropyl-biphenyl-4-yloxy)-3-methyl-butyl]-benzoylamino}-propionic acid;
3-{4-[1-(2-Chloro-4′-isopropyl-biphenyl-4-yloxy)-3-methyl-butyl]-benzoylamino}-propionic acid;
3-{4-[1-(2-Chloro-4′-trifluoromethyl-biphenyl-4-yloxy)-3-methyl-butyl]-benzoylamino}-propionic acid;
3-(4-{3-Methyl-1-[5-methyl-6-(4-trifluoromethyl-phenyl)-pyridin-3-yloxy]-butyl}-benzoylamino)-propionic acid;
3-(4-{1[6-(4-Isopropyl-phenyl)-5-methyl-pyridin-3-yloxyl -3-methyl-butyl]-benzoylamino}-propionic acid;
3-{4-[1-(4′-tert-Butyl-biphenyl-3-yloxy)-3-methyl-butyl]-benzoylamino}-propionic acid;
3-{4-[3-Methyl-1-(4′-trifluoromethyl-biphenyl-3-yloxy)-butyl]-benzoylamino}-propionic acid;
3-{4-[1-(4′-Isopropyl-biphenyl-3-yloxy)-3-methyl-butyl]-benzoylamino}-propionic acid;
3-{4-[3-Methyl-1-(4′-trifluoromethoxy-biphenyl-3-yloxy)-butyl]-benzoylamino}-propionic acid;
3-{4-[3-Methyl-1-(6-methyl-4′-trifluoromethyl-biphenyl-3-yloxy)-butyl]-benzoylamino}-propionic acid;
3-{4-[1-(4′-tert-Butyl-6-methyl-biphenyl-3-yloxy)-3-methyl-butyl]-benzoylamino}-propionic acid
3-{4-[1-(2-Hydroxymethyl-4′-isopropyl-biphenyl-4-yloxy)-3-methyl-butyl]-benzoylamino}-propionic acid;
3-{4-[1-(2-Formyl-4′-isopropyl-biphenyl-4-yloxy)-3-methyl-butyl]-benzoylamino}-propionic acid
3-{4-[1-(2-Hydroxymethyl-4′-isopropyl-biphenyl-4-yloxy)-3-methyl-butyl]-benzoylamino}-propionic acid;
3-(4-{1-[2-(Hydroxyimino-methyl)-4′-isopropyl-biphenyl-4-yloxy]-3-methyl-butyl}-benzoylamino)-propionic acid;
3-{4-[1-(4′-Isopropyl-2-morpholin-4-ylmethyl-biphenyl-4-yloxy)-3-methyl-butyl]-benzoylamino}-propionic acid;
3-(4-{3,3-Dimethyl-1-[5-methyl-1-oxy-6-(4-trifluoromethoxy-phenyl)-pyridin-3-yloxy]-butyl}-benzoylamino)-propionic acid;
3-(4-{3,3-Dimethyl-1-[5-methyl-1-oxy-6-(4-trifluoromethoxy-phenyl)-pyridin-3-yloxy]-butyl}-benzoylamino)-propionic acid;
2-Hydroxy-3-{4-[1-(4′-isopropyl-2,6-dimethyl-biphenyl-4-yloxy)-2-methyl-propyl]-benzoylamino}-propionic acid;
3-{4-[1-(2,6-Dimethyl-4′-trifluoromethyl-biphenyl-4-yloxy)-2-methyl-propyl]-benzoylamino}-2-hydroxy-propionic acid;
3-{4-[1-(2,6-Dimethyl-4′-trifluoromethyl-biphenyl-4-yloxy)-2-methyl-propyl]-benzoylamino}-2-hydroxy-propionic acid;
3-{4-[1-(2,6-dimethyl-4′-trifluoromethyl-biphenyl-4-ylsulfanyl)-5,5,5-trifluoro-pentyl]-benzoylamino}-propionic acid;
3-{4-[1-(2,4,6-tri-t-butyl-phenoxy)-5,5,5-trifluoro-pentyl]-benzoylamino}-propionic acid;
3-(4-{5,5,5-trifluoro-1-[5-methyl-6-(4-trifluoromethyl-phenyl)-pyridin-3-yloxy]-pentyl}-benzoylamino)-propionic acid;
3-(4-{1-[6-(4-trifluoromethyl-phenyl)-pyridin-3-yloxy]-pentyl}-benzoylamino)-propionic acid;
3-{4-[1-(4′-tert-butyl-2,6-dimethyl-biphenyl-4-ylsulfanyl)-5,5,5-trifluoro-pentyl]-benzoylamino}-propionic acid;
3-{4-[1-(4-bromo-3,5-dimethyl-phenylsulfanyl)-5,5,5-trifluoro-pentyl]-benzoylamino}-propionic acid;
3-(4-{5,5,5-trifluoro-1-[5-methyl-6-(4-trifluoromethyl-phenyl)-pyridin-3-yloxy]-pentyl}-benzoylamino)-propionic acid;
3-(4-{4,4,4-trifluoro-1-[5-methyl-6-(4-trifluoromethyl-phenyl)-pyridin-3-yloxy]-butyl}-benzoylamino)-propionic acid;
3-{4-[1-(2,6-dimethyl-4-trifluoromethyl-biphenyl-4-ylsulfanyl)-3-methyl-butyl]-benzoylamino}-propionic acid;
3-{4-[1-(4′-tert-butyl-2,6-dimethyl-biphenyl-4-ylsulfanyl)-3-methyl-butyl]-benzoylamino}-propionic acid;
3-{4-[1-(4′-tert-butyl-2,6-dimethyl-biphenyl-4-ylsulfanyl)-4,4,4-trifluoro-butyl]-benzoylamino}-propionic acid;
3-{4-[1-(4-bromo-3,5-dimethyl-phenylsulfanyl)-4,4,4-trifluoro-butyl]-benzoylamino}-propionic acid;
3-{4-[1-(4-bromo-3,5-dimethyl-phenylsulfanyl)-3-methyl-butyl]-benzoylamino}-propionic acid;
3-{4-[1-(2,6-dimethyl-4′-trifluoromethyl-biphenyl-4-yloxy)-5,5,5-trifluoro-pentyl]-benzoylamino}-propionic acid;
3-{4-[4,4,4-trifluoro-1-(4′-isopropyl-2,6-dimethyl-biphenyl-4-yloxy)-butyl]-benzoylamino}-propionic acid;
3-{4-[1-(2,6-dimethyl-4′-trifluoromethyl-biphenyl-4-ylsulfanyl)-4,4,4-trifluoro-butyl]-benzoylamino}-propionic acid;
3-{4-[1-(4′-tert-butyl-2,6-dimethyl-biphenyl-4-ylsulfanyl)-4,4,4-trifluoro-butyl]-benzoylamino}-propionic acid;
3-{4-[1-(4′-trifluoromethyl-2,6-dimethyl-biphenyl-4-ylsulfanyl)-3-methyl-butyl]-benzoylamino}-propionic acid;
3-{4-[1-(4′-tert-butyl-2,6-dimethyl-biphenyl-4-ylsulfanyl)-3-methyl-butyl]-benzoylamino}-propionic acid;
3-{4-[1-(4-bromo-3,5-dimethyl-phenylsulfanyl)-4,4,4-trifluoro-butyl]-benzoylamino}-propionic acid;
3-{4-[1-(4-bromo-3,5-dimethyl-phenylsulfanyl)-3-methyl-butyl]-benzoylamino}-propionic acid;
3-{4-[1-(2,6-dimethyl-4′-trifluoromethyl-biphenyl-4-yloxy)-heptyl]-benzoylamino}-propionic acid;
3-{4-[3-methyl-1-(2,2′,4′-trichloro-biphenyl-4-yloxy)-butyl]-benzoylamino}-propionic acid;
3-{4-[1-(2,6-dimethyl-4′-trifluoromethyl-biphenyl-4-yloxy)-4,4,4-trifluoro-butyl]-benzoylamino}-propionic acid;
3-{4-[1-(4′-tert-butyl-2,6-dimethyl-biphenyl-4-yloxy)-4,4,4-trifluoro-butyl]-benzoylamino}-propionic acid;
3-{4-[5,5,5-trifluoro-1-(4′-isopropyl-2,6-dimethyl-biphenyl-4-yloxy)-pentyl]-benzoylamino}-propionic acid;
3-{4[5,5,5-trifluoro-1-(4′-isopropyl-2,6-dimethyl-biphenyl-4-yloxy)-pentyl]-benzoylamino}-propionic acid;
3-{4-[1-(2,6-dimethyl-4′-trifluoromethyl-biphenyl-4-yloxy)-5-methyl-hexyl]-benzoylamino}-propionic acid;
3-{4-[5-methyl-1-(4′-trifluoromethyl-biphenyl-4-yloxy)-hexyl]-benzoylamino}-propionic acid;
3-(4-{1-[6-(4-tert-butyl-phenyl)-pyridin-3-yloxy]-5-methyl-hexyl}-benzoylamino)-propionic acid;
3-(4-{5-methyl-1-[6-(4-trifluoromethyl-phenyl)-pyridin-3-yloxy]-hexyl}-benzoylamino)-propionic acid;
3-{4-[1-(6-chloro-pyridin-3-yloxy)-5-methyl-hexyl]-benzoylamino}-propionic acid;
3-{4-[1-(2,6-dimethyl-4′-trifluoromethyl-biphenyl-4-yloxy)-5-methyl-hexyl]-benzoylamino}-propionic acid;
3-(4-{1-[6-(4-tert-butyl-phenyl)-pyridin-3-yloxy]-5 -methyl-hexyl}-benzoylamino)-propionic acid;
3-{4-[5-methyl-1-(4′-trifluoromethyl-biphenyl-4-yloxy)-hexyl]-benzoylamino}-propionic acid;
3-{4-[1-(4′-tert-butyl-biphenyl-4-yloxy)-5-methyl-hexyl]-benzoylamino}-propionic acid;
3-(4-{1-[6-(4-tert-butyl-phenyl)-pyridin-3-yloxy]-5,5,5-trifluoro-pentyl}-benzoylamino)-propionic acid;
3-(4-{1-[6-(4-tert-butyl-phenyl)-pyridin-3-yloxy]-4,4,4-trifluoro-butyl}-benzoylamino)-propionic acid;
3-{4-[(4′-tert-butyl-2,6-dimethyl-biphenyl-4-yloxy)-cyclohexyl-methyl]-benzoylamino}-propionic acid;
3-{4-[1-(4′-tert-butyl-2,6-dimethyl-biphenyl-4-yloxy)-4,4,4-trifluoro-butyl]-benzoylamino}-propionic acid;
3-{4-[4,4,4-trifluoro-1-(4′-trifluoromethyl-biphenyl-4-yloxy)-butyl]-benzoylamino}-propionic acid;
3-(4-{4,4,4-trifluoro-1-[6-(4-isopropyl-phenyl)-pyridin-3-yloxy]-butyl}-benzoylamino)-propionic acid;
3-(4-{5,5,5-trifluoro-1-[6-(4-trifluoromethyl-phenyl)-pyridin-3-yloxy]-pentyl}-benzoylamino)-propionic acid;
3-(4-{[6-(4-tert-butyl-phenyl)-pyridin-3-yloxy)-cyclohexyl-methyl]-benzoylamino}-propionic acid;
3-(4-{5,5,5-trifluoro-1-[6-(4-isopropyl-phenyl)-pyridin-3-yloxy]-pentyl}-benzoylamino)-propionic acid;
3-{4-[1-(2,6-dimethyl-4′-trifluoromethyl-biphenyl-4-yloxy)-4,4,4-trifluoro-butyl]-benzoylamino}-propionic acid;
3-(4-{cyclohexyl-[6-(4-isopropyl-phenyl)-pyridin-3-yloxy]-methyl}-benzoylamino)-propionic acid;
3-{4-[cyclohexyl-(4′-isopropyl-2,6-dimethyl-biphenyl-4-yloxy)-methyl]-benzoylamino}-propionic acid;
3-{4-[4,4,4-trifluoro-1-(4′-isopropyl-2,6-dimethyl-biphenyl-4-yloxy)-butyl]-benzoylamino}-propionic acid;
3-{4-[cyclohexyl-(4′-trifluoromethyl-biphenyl-4-yloxy)-methyl]-benzoylamino}-propionic acid;
3-{4-[5,5,5-trifluoro-1-(4′-isopropyl-biphenyl-4-yloxy)-pentyl]-benzoylamino}-propionic acid;
3-{4-[(4′-tert-butyl-biphenyl-4-yloxy)-cyclohexyl-methyl]-benzoylamino}-propionic acid;
3-{4-[5,5,5-trifluoro-1-(2′-3′-fluoro-4′-trifluoromethyl-biphenyl-4-yloxy)-pentyl]-benzoylamino}-propionic acid;
3-{4-[cyclohexyl-(4′-trifluoromethyl-biphenyl-4-yloxy)-methyl]-benzoylamino}-propionic acid;
3-{4-[5,5,5-trifluoro-1-(4′-isopropyl-biphenyl-4-yloxy)-pentyl]-benzoylamino}-propionic acid;
3-{4-[1-(4′-ethyl-biphenyl-4-yloxy)-5,5,5-trifluoro-pentyl]-benzoylamino}-propionic acid;
3-{4-[5,5,5-trifluoro-1-(3′-fluoro-4′-trifluoromethyl-biphenyl-4-yloxy)-pentyl]-benzoylamino}-propionic acid;
3-{4-[1-(2,4,6-triisopropyl-phenoxy)-5,5,5-trifluoro-pentyl]-benzoylamino}-propionic acid;
3-{4-[1-(2,3,4,5,6-pentamethyl-phenoxy)-5,5,5-trifluoro-pentyl]-benzoylamino}-propionic acid;
3-{4-[1-(2,4,6-tri-t-butyl-phenoxy)-5,5,5-trifluoro-pentyl]-benzoylamino}-propionic acid;
3-{4-[1-(3,5-dimethyl-phenoxy)-5,5,5-trifluoro-pentyl]-benzoylamino}-propionic acid;
3-{4-[1-(4′-tert-butyl-2,6-dimethyl-biphenyl-4-yloxy)-5,5,5-trifluoro-pentyl]-benzoylamino}-propionic acid;
3-{4-[1-(4-ethyl-3,5-dimethyl-phenoxy)-5,5,5-trifluoro-pentyl]-benzoylamino}-propionic acid;
3-{4-[1-(2,6-dimethyl-4′-trifluoromethyl-biphenyl-4-yloxy)-5,5,5-trifluoro-pentyl]-benzoylamino}-propionic acid;
3-(4-{1-[4-(4-methyl-pentyloxy)-phenoxy]-heptyl}-benzoylamino)-propionic acid;
3-{4-[1-(4-pentyloxy-phenoxy)-heptyl]-benzoylamino}-propionic acid;
3-{4-[5,5,5-trifluoro-1-(4′-trifluoromethyl-biphenyl-4-yloxy)-pentyl]-benzoylamino}-propionic acid;
3-{4-[1-(4′-tert-butyl-2,6-dimethyl-biphenyl-4-yloxy)-5,5,5-trifluoro-pentyl]-benzoylamino}-propionic acid;
3-{4-[1-(4-bromo-3,5 -dimethyl-phenoxy)-5,5,5-trifluoro-pentyl]-benzoylamino}-propionic acid;
3-{4-[1-(4′-tert-butyl-biphenyl-4-yloxy)-5,5,5-trifluoro-pentyl]-benzoylamino}-propionic acid;
3-(4-{1-[6-(4-trifluoromethyl-phenyl)-pyridin-3-yloxy]-hexyl}-benzoylamino)-propionic acid;
3-(4-{1-[6-(4-trifluoromethyl-phenyl)-pyridin-3-yloxy]-pentyl}-benzoylamino)-propionic acid;
3-{4-[5,5,5-trifluoro-1-(4′-trifluoromethyl-biphenyl-4-yloxy)-pentyl]-benzoylamino}-propionic acid;
3-{4-[1-(4′-tert-butyl-biphenyl-4-yloxy)-5,5,5-trifluoro-pentyl]-benzoylamino}-propionic acid;
3-{4-[1-(4-bromo-phenoxy)-5,5,5 -trifluoro-pentyl]-benzoylamino}-propionic acid;
3-{4-[1-(4-hydroxy-phenoxy)-heptyl]-benzoylamino}-propionic acid;
3-{4-[1-(2,6-Dimethyl-4′-trifluoromethyl-biphenyl-4-yloxy)-2-methyl-propyl]-benzoylamino}-propionic acid;
3-{4-[1-(2,6-Dimethyl-4′-trifluoromethoxy-biphenyl-4-yloxy)-heptyl]-benzoylamino}-propionic acid;
(R,S)-5-[1-(4′-tert-Butyl-2,6-dimethyl-biphenyl-4-ylsulfanyl)-2-methyl-propyl]-thiophene-2-carboxylic acid (2H-tetrazol-5-ylmethyl)-amide;
(±)-5-[1-(4′-tert-Butyl-2,6-dimethyl-biphenyl-4-ylsulfanyl)-propyl]-thiophene-2-carboxylic acid (2H-tetrazol-5-ylmethyl)-amide;
(±)-5-[1-(4′-tert-Butyl-2,6-dimethyl-biphenyl-4-ylsulfanyl)-butyl]-thiophene-2-carboxylic acid (2H-tetrazol-5-ylmethyl)-amide;
(±)-5-[1-(4′-tert-Butyl-2,6-dimethyl-biphenyl-4-ylsulfanyl)-pentyl]-thiophene-2-carboxylic acid (2H-tetrazol-5-ylmethyl)-amide;
(±)-5-[1-(4′-tert-Butyl-2,6-dimethyl-biphenyl-4-ylsulfanyl)-3-methyl-butyl]-thiophene-2-carboxylic acid (2H-tetrazol-5-ylmethyl)-amide;
((±)-5-[1-(4′-tert-Butyl-2,6-dimethyl-biphenyl-4-ylsulfanyl)-3,3-dimethyl-butyl]-thiophene-2-carboxylic acid (2H-tetrazol-5-ylmethyl)-amide;
(±)-5-[1-(4′-tert-Butyl-2,6-dimethyl-biphenyl-4-ylsulfanyl)-2,2-dimethyl-propyl]-thiophene-2-carboxylic acid (2H-tetrazol-5-ylmethyl)-amide;
(±)-5-[1-(2,6-Dimethyl-4′-trifluoromethyl-biphenyl-4-ylsulfanyl)-butyl]-thiophene-2-carboxylic acid (1H-tetrazol-5-ylmethyl)-amide;
(±)-5-[1-(2,6-Dimethyl-4′-trifluoromethyl-biphenyl-4-ylsulfanyl)-2-methyl-propyl]-thiophene-2-carboxylic acid (1H-tetrazol-5-ylmethyl)-amide;
(±)-5-[1-(2,6-Dimethyl-4′-trifluoromethyl-biphenyl-4-ylsulfanyl)-2,2-dimethyl-propyl]-thiophene-2-carboxylic acid (1H-tetrazol-5-ylmethyl)-amide;
(±)-5-[1-(2,6-Dimethyl-4′-trifluoromethyl-biphenyl-4-ylsulfanyl)-3-methyl-butyl]-thiophene-2-carboxylic acid (1H-tetrazol-5-ylmethyl)-amide;
(±)-5-[1-(2,6-Dimethyl-4′-trifluoromethyl-biphenyl-4-ylsulfanyl)-3,3-dimethyl-butyl]-thiophene-2-carboxylic acid (1H-tetrazol-5-ylmethyl)-amide;
(±)-5-[1-(2,6-Dimethyl-4′-trifluoromethyl-biphenyl-4-ylsulfanyl)-pentyl]-thiophene-2-carboxylic acid (1H-tetrazol-5-ylmethyl)-amide;
5-[1-(4′-tert-Butyl-2,6-dimethyl-biphenyl-4-ylsulfanyl)-2,2-dimethyl-propyl]-thiophene-2-carboxylic acid (2H-tetrazol-5-ylmethyl)-amide;
5-[1-(4′-tert-Butyl-2,6-dimethyl-biphenyl-4-ylsulfanyl)-propyl]-thiophene-2-carboxylic acid (2H-tetrazol-5-ylmethyl)-amide;
5-[1-(2,6-Dimethyl-4′-trifluoromethyl-biphenyl-4-ylsulfanyl)-butyl]-thiophene-2-carboxylic acid (1H-tetrazol-5-ylmethyl)-amide;
5-[1-(2,6-Dimethyl-4′-trifluoromethyl-biphenyl-4-ylsulfanyl)-2-methyl-propyl]-thiophene-2-carboxylic acid (1H-tetrazol-5-ylmethyl)-amide;
5-[1-(2,6-Dimethyl-4′-trifluoromethyl-biphenyl-4-ylsulfanyl)-2,2-dimethyl-propyl]-thiophene-2-carboxylic acid (1H-tetrazol-5-ylmethyl)-amide;
5-[1-(2,6-Dimethyl-4′-trifluoromethyl-biphenyl-4-ylsulfanyl)-3-methyl-butyl]-thiophene-2-carboxylic acid (1H-tetrazol-5-ylmethyl)-amide;
5-[1-(2,6-Dimethyl-4′-trifluoromethyl-biphenyl-4-ylsulfanyl)-3,3 -dimethyl-butyl]-thiophene-2-carboxylic acid (1H-tetrazol-5-ylmethyl)-amide;
5-[1-(2,6-Dimethyl-4′-trifluoromethyl-biphenyl-4-ylsulfanyl)-pentyl]-thiophene-2-carboxylic acid (1H-tetrazol-5-ylmethyl)-amide;
4-[1-(2,6-Dimethyl-4′-trifluoromethyl-biphenyl-4-yloxy)-3,3-dimethyl-butyl]-N-(1H-tetrazol-5-ylmethyl)-benzamide;
4-[1-(-4′-Isopropyl-2,6-dimethyl-biphenyl-4-yloxy)-3,3-dimethyl-butyl]-N-(1H-tetrazol-5-ylmethyl)-benzamide;
4-[1-(2,6-Dimethyl-4′-trifluoromethyl-biphenyl-4-yloxy)-2-methyl-propyl]-N-(1H-tetrazol-5-ylmethyl)-benzamide;
4-[1-(2,6-Dimethyl-4′-trifluoromethyl-biphenyl-4-yloxy)-4,4-dimethyl-pentyl]-N-(1H-tetrazol-5-ylmethyl)-benzamide;
4-[1-(2,6-Dimethyl-4′-trifluoromethyl-biphenyl-4-yloxy)-4,4-dimethyl-pentyl]-N-(1H-tetrazol-5-ylmethyl)-benzamide;
4-[1-(2,6-Dimethyl-4′-trifluoromethyl-biphenyl-4-yloxy)-3-methyl-butyl]-N-(1H-tetrazol-5-ylmethyl)-benzamide;
4-[1-(4′-tert-Butyl-2,6-dimethyl-biphenyl-4-yloxy)-3-methyl-butyl]-N-(1H-tetrazol-5-ylmethyl)-benzamide;
4-[1-(2,6-Dimethyl-4′-trifluoromethyl-biphenyl-4-yloxy)-5,5,5-trifluoro-pentyl]-N-(1H-tetrazol-5-ylmethyl)-benzamide;
4-[1-(4′-tert-Butyl-2,6-dimethyl-biphenyl-4-yloxy)-4,4,4-trifluoro-butyl]-N-(1H-tetrazol-5-ylmethyl)-benzamide;
4-[Cyclobutyl-(2,6-dimethyl-4′-trifluoromethyl-biphenyl-4-yloxy)-methyl]-N-(1H-tetrazol-5-ylmethyl)-benzamide;
4-[Cyclopentyl-(2,6-dimethyl-4′-trifluoromethyl-biphenyl-4-yloxy)-methyl]-N-(1H-tetrazol-5-ylmethyl)-benzamide;
4-[1-(4′-Isopropyl-2,6-dimethyl-biphenyl-4-yloxy)-2-methyl-propyl]-N-(1H-tetrazol-5-yl)-benzamide;
4-[1-(2,6-Dimethyl-4′-trifluoromethyl-biphenyl-4-yloxy)-2-methyl-propyl]-N-(1H-tetrazol-5-yl)-benzamide;
4-[1-(2,6-Dimethyl-4′-trifluoromethyl-biphenyl-4-yloxy)-3,3-dimethyl-butyl]-N-(1H-tetrazol-5-yl)-benzamide;
4-[1-(4′-Isopropyl-2,6-dimethyl-biphenyl-4-yloxy)-3,3-dimethyl-butyl]-N-(1H-tetrazol-5-yl)-benzamide,
4-[1-(2,6-Dimethyl-4′-trifluoromethyl-biphenyl-4-yloxy)-4,4-dimethyl-pentyl]-N-(1H-tetrazol-5-yl)-benzamide;
4-[1-(4′-tert-Butyl-2,6-dimethyl-biphenyl-4-yloxy)-3-methyl-butyl]-N-(1H-tetrazol-5-yl)-benzamide;
4-[1-(2,6-Dimethyl-4′-trifluoromethyl-biphenyl-4-yloxy)-3-methyl-butyl]-N-(1H-tetrazol-5-yl)-benzamide;
3-{4-[1-(4′-tert-Butyl-biphenyl-4-ylmethyl)-3-methyl-butyl]-benzoylamino}-propionic acid
3-{4-[1-(2,6-Dimethyl-4′-trifluoromethyl-biphenyl-4-ylmethyl)-3-methyl-butyf]-benzoylamino}-propionic acid;
3-{4-[1-(4′-tert-Butyl-biphenyl-4-ylmethyl)-heptyl]-benzoylamino}-propionic acid;
3-{4-[1-(4′-Trifluoromethyl-biphenyl-4-ylmethyl)-heptyl]-benzoylamino}-propionic acid;
3-[4-(1-Benzyl-heptyl)-benzoylamino]-propionic acid;
3-{4-[1-(4′-tert-Butyl-biphenyl-4-ylmethyl)-heptyl]-benzoylamino}-propionic acid;
3-{4-[1-(4′-tert-Butyl-biphenyl-4-ylmethyl)-3-methyl-butyl]-benzoylamino}-propionic acid;
3-{4-[1-(2,6-Dimethyl-4′-trifluoromethyl-biphenyl-4-ylmethyl)-3-methyl-butyl]-benzoylamino}-propionic acid;
3-{4-[1-Allyl-1-(4′-trifluoromethyl-biphenyl-4-yloxymethyl)-but-3-enyl]-benzoylamino}-propionic acid;
3-{4-[1-Allyl-1-(2,6-dimethyl-4′-trifluoromethyl-biphenyl-4-yloxymethyl)-but-3-enyl]-benzoylamino}-propionic acid;
3-{4-[2-(2,6-Dimethyl-4′-trifluoromethyl-biphenyl-4-yloxy)-1,1-dimethyl-ethyl]-benzoylamino}-propionic acid;
3-{4-[1-(2,6-Dimethyl-4′-trifluoromethyl-biphenyl-4-yloxymethyl)-1-ethyl-propyl]-benzoylamino}-propionic acid;
3-{4-[1-(2,6-dimethyl-4′-trifluoromethyl-biphenyl-4-yloxymethyl)-cyclopent-3-enyl]-benzoylamino}-propionic acid;
3-{4-[1-(2,6-Dimethyl-4′-trifluoromethyl-biphenyl-4-yloxymethyl)-cyclopentyl]-benzoylamino}-propionic acid;
3-{4-[1-Propyl-1-(4′-trifluoromethyl-biphenyl-4-yloxymethyl)-butyl]-benzoylamino}-propionic acid;
3-{4-[1-(4′-Trifluoromethyl-biphenyl-4-yloxymethyl)-heptyl]-benzoylamino}-propionic acid;
3-{4-[1-(4′-tert-Butyl-biphenyl-4-yloxymethyl)-heptyl]-benzoylamino}-propionic acid;
3-{4-[1-(4′-tert-Butyl-2,6-dimethyl-biphenyl-4-yloxymethyl)-3-methyl-butyl]-benzoylamino}-propionic acid;
3-{4-[1-(4′-tert-Butyl-biphenyl-4-yloxymethyl)-butyl]-benzoylamino}-propionic acid;
3-{4-[1-(4′-Trifluoromethyl-biphenyl-4-yloxymethyl)-butyl]-benzoylamino}-propionic acid;
3-{4-[1-(4′-tert-Butyl-biphenyl-4-yloxymethyl)-5,5,5-trifluoro-pentyl]-benzoylamino}-propionic acid;
3-{4-[1-(2,6 -Dimethyl-4′-trifluoromethyl-biphenyl-4-yloxymethyl)-3-methyl-butyl]-benzoylamino}-propionic acid;
3-{4-[1-(4′-Trifluoromethyl-biphenyl-4-yloxymethyl)-heptyl]-benzoylamino}-propionic acid;
3-{4-[1-(4′-tert-Butyl-2,6-dimethyl-biphenyl-4-yloxymethyl)-3-methyl-butyl]-benzoylamino}-propionic acid;
3-{4-[1-(2,6-Dimethyl-4′-trifluoromethyl-biphenyl-4-yloxymethyl)-3-methyl-butyl]-benzoylamino}-propionic acid;
3-{4-[1-(2,6-Dimethyl-4′-trifluoromethyl-biphenyl-4-yloxymethyl)-2-methyl-propyl]-benzoylamino}-propionic acid;
3-{4-[1-(4′-tert-Butyl-2,6-dimethyl-biphenyl-4-yloxymethyl)-2-methyl-propyl]-benzoylamino}-propionic acid;
3-(4-{1-[6-(4-Trifluoromethyl-phenyl)-pyridin-3-yloxymethyl]-butyl}-benzoylamino)-propionic acid;
3-{4-[1-(2,6-Dimethyl-4′-trifluoromethyl-biphenyl-4-yloxymethyl)-4,4,4-trifluoro-butyl]-benzoylamino}-propionic acid;
3-{4-[1-(2,6-Dimethyl-4′-trifluoromethyl-biphenyl-4-yloxymethyl)-2-methyl-propyl]-benzoylamino}-propionic acid;
3-{4-[1-(4′-tert-Butyl-2,6-dimethyl-biphenyl-4-yloxymethyl)-2-methyl-propyl]-benzoylamino}-propionic acid;
3-{4-[1-(2,6-Dimethyl-4′-trifluoromethyl-biphenyl-4-yloxymethyl)-4,4,4-trifluoro-butyl]-benzoylamino}-propionic acid;
3-{4-[1-(4′-tert-Butyl-biphenyl-4-ylamino)-3-methyl-butyl]-benzoylamino}-propionic acid;
3-{4-[3-Methyl-1-(4′-trifluoromethyl-biphenyl-4-ylamino)-butyl]-benzoylamino}-propionic acid;
3-{4-[1-(2,6-Dimethyl-4′-trifluoromethyl-biphenyl-4-ylamino)-3-methyl-butyl]-benzoylamino}-propionic acid;
3-{4-[1-(4′-Cyano-biphenyl-4-ylamino)-3-methyl-butyl]-benzoylamino}-propionic acid;
3-{4-[1-(4′-Cyano-biphenyl-4-ylamino)-hexyl]-benzoylamino}-propionic acid;
3-{4-[1-(2-Methoxy-biphenyl-4-ylamino)-hexyl]-benzoylamino}-propionic acid;
3-{4-[1-(4′-Cyano-biphenyl-4-ylamino)-3-methyl-butyl]-benzoylamino}-propionic acid;
3-{4-[1-(4′-tert-Butyl-biphenyl-4-ylamino)-3-methyl-butyl]-benzoylamino}-propionic acid;
3-{4-[3-Methyl-1-(4′-trifluoromethyl-biphenyl-4-ylamino)-butyl]-benzoylamino}-propionic acid;
3-{4-[6-(4-Trifluoromethyl-phenyl)-pyridin-3-ylmethoxyl]-benzoylamino}-propionic acid;
R,S)-3-(4-{1-[6-(4-Trifluoromethyl-phenyl)-pyridin-3-yl]-ethoxy}-benzoylamino)-propionic acid;
(R,S)-3-(4-{2-Cyclohexyl-1-[6-(4-trifluoromethyl-phenyl)-pyridin-3-yl]-ethoxy}-benzoylamino)-propionic acid;
(R,S)-3-(4-{1-[5-(4-Trifluoromethyl-phenyl)-pyridin-2-yl]-propoxy}-benzoylamino)-propionic acid;
(R,S)-3-(4-{1-[6-(4-Trifluoromethyl-phenyl)-pyridin-3-yl]-heptyloxy}-benzoylamino)-propionic acid;
(R,S)-3-(4-{2-Cyclohexyl-1-[6-(4-trifluoromethyl-phenyl)-pyridin-3-yl]-ethylsulfanyl}-benzoylamino)-propionic acid;
(R,S)-3-(4-{1-[6-(4-Trifluoromethyl-phenyl)-pyridin-3-yl]-heptylsulfanyl}-benzoylamino)-propionic acid;
(R,S)-3-(4-{1-[6-(4-Trifluoromethyl-phenyl)-pyridin-3-yl]-propoxy}-benzoylamino)-propionic acid;
(R) and (S)-3-(4-{2-Cyclohexyl-1-[6-(4-trifluoromethyl-phenyl)-pyridin-3-yl]-ethoxy}-benzoylamino)-propionic acid;
(R) and (S)-3-(4-{1-[6-(4-Trifluoromethyl-phenyl)-pyridin-3-yl]-heptyloxy}-benzoylamino)-propionic acid;
(R) and (S)-3-(4-{1-[6-(4-Trifluoromethyl-phenyl)-pyridin-3-yl]-propoxy}-benzoylamino)-propionic acid;
3-[4-(3-Methyl-4′-trifluoromethyl-biphenyl-4-ylmethoxy)-benzoylamino]-propionic acid;
3-[4-(3-Methyl-3′,5′-bis-trifluoromethyl-biphenyl-4-ylmethoxy)-benzoylamino]-propionic acid;
3-[4-(3-Methyl-biphenyl-4-ylmethoxy)-benzoylamino]-propionic acid;
(R,S)-3-(4-{(4-tert-Butyl-phenyl)-[6-(4-trifluoromethyl-phenyl)-pyridin-3-yl]-methoxy}-benzoylamino)-propionic acid;
(R,S)-3-(4-{1-[6-(4-Trifluoromethyl-phenyl)-pyridin-3-yl]-hexyloxy}-benzoylamino)-propionic acid;
(R,S)-3-(4-{1-[6-(4-Trifluoromethyl-phenyl)-pyridin-3-yl]-butoxy}-benzoylamino)-propionic acid;
(R,S)-3-(4-{1-[6-(4-Trifluoromethyl-phenyl)-pyridin-3-yl]-pentyloxy}-benzoylamino)-propionic acid;
(R,S)-3-(4-{3-Cyclohexyl-1-[6-(4-trifluoromethyl-phenyl)-pyridin-3-yl]-propoxy}-benzoylamino)-propionic acid;
(R,S)-3-(4-{1-[6-(4-Trifluoromethyl-phenyl)-pyridin-3-yl]-nonyloxy}-benzoylamino)-propionic acid;
(R,S)-3-(4-{4,4-Dimethyl-1-[6-(4-trifluoromethyl-phenyl)-pyridin-3-yl]-pentyloxy}-benzoylamino)-propionic acid;
(R,S)-3-(4-{4,4,4-Trifluoro-1-[6-(4-trifluoromethyl-phenyl)-pyridin-3-yl]-butoxy}-benzoylamino)-propionic acid;
(R,S)-3-(4-{5,5,5-Trifluoro-1-[6-(4-trifluoromethyl-phenyl)-pyridin-3-yl]-pentyloxy}-benzoylamino)-propionic acid;
(R,S)-3-(4-{5,5,5-Trifluoro-1-[6-(4-trifluoromethyl-phenyl)-pyridin-3-yl]-pentylsulfanyl}-benzoylamino)-propionic acid;
(R,S)-3-(4-{3,3-Dimethyl-1-[6-(4-trifluoromethyl-phenyl)-pyridin-3-yl]-butoxy}-benzoylamino)-propionic acid;
3-(4-{(4-tert-Butyl-phenyl)-[6-(4-trifluoromethyl-phenyl)-pyridin-3-yl]-methoxy}-benzoylamino)-propionic acid;
3-(4-{1-[6-(4-Trifluoromethyl-phenyl)-pyridin-3-yl]-hexyloxy}-benzoylamino)-propionic acid;
3-(4-{1-[6-(4-Trifluoromethyl-phenyl)-pyridin-3-yl]-butoxy}-benzoylamino)-propionic acid;
3-(4-{(3-Cyclohexyl-1-[6-(4-trifluoromethyl-phenyl)-pyridin-3-yl]-propoxy}-benzoylamino)-propionic acid;
3-(4-{4,4-Dimethyl-1-[6-(4-trifluoromethyl-phenyl)-pyridin-3-yl]-pentyloxy}-benzoylamino)-propionic acid;
3-(4-{4,4,4-Trifluoro-1-[6-(4-trifluoromethyl-phenyl)-pyridin-3-yl]-butoxy}-benzoylamino)-propionic acid;
3-(4-{5,5,5 -Trifluoro-1-[6-(4-trifluoromethyl-phenyl)-pyridin-3-yl]-pentyloxy}-benzoylamino)-propionic acid;
3-(4-{1-[6-(2-Trifluoromethyl-phenyl)-pyridin-3-yl]-hexyloxy}-benzoylamino)-propionic acid;
(R,S)-3-(4-{1-[6-(3-Trifluoromethyl-phenyl)-pyridin-3-yl]-hexyloxy}-benzoylamino)-propionic acid;
(R,S)-3-(4-{1-[6-(4-Fluoro-phenyl)-pyridin-3-yl]-hexyloxy}-benzoylamino)-propionic acid;
(R,S)-3-{4-[1-(6-p-Tolyl-pyridin-3-yl)-hexyloxy]-benzoylamino}-propionic acid;
(R,S)-3-{4-[1-(6-Phenyl-pyridin-3-yl)-hexyloxy]-benzoylamino}-propionic acid;
(R,S)-3-(4-{1-[6-(4-Trifluoromethoxy-phenyl)-pyridin-3-yl]-hexyloxy}-benzoylamino)-propionic acid;
(R,S)-3-(4-{1-[6-(4-Methoxy-phenyl)-pyridin-3-yl]-hexyloxy}-benzoylamino)-propionic acid;
(R,S)-3-(4-{1-[6-(4-Isobutyl-phenyl)-pyridin-3-yl]-hexyloxy}-benzoylamino)-propionic acid;
(R,S)-3-(4-{1-[6-(4-Cyclohexyl-phenyl)-pyridin-3-yl]-hexyloxy}-benzoylamino)-propionic acid;
(R,S)-3-(4-{1-[6-(4-Ethyl-phenyl)-pyridin-3-yl]-hexyloxy}-benzoylamino)-propionic acid;
(R,S)-3-(4-{1-[6-(4-Pentyl-phenyl)-pyridin-3-yl]-hexyloxy}-benzoylamino)-propionic acid;
(R,S)-3-(4-{1-[6-(4-tert-Butyl-phenyl)-pyridin-3-yl]-hexyloxy}-benzoylamino)-propionic acid;
(R,S)-3-(4-{4,4-Dimethyl-1-[6-(2-trifluoromethyl-phenyl)-pyridin-3-yl]-pentyloxy}-benzoylamino)-propionic acid;
(R,S)-3-(4-{4,4-Dimethyl-1-[6-(3-trifluoromethyl-phenyl)-pyridin-3-yl]-pentyloxy}-benzoylamino)-propionic acid;
(R,S)-3-(4-{1-[6-(3,5-Bis-trifluoromethyl-phenyl)-pyridin-3-yl]-4,4-dimethyl-pentyloxy}-benzoylamino)-propionic acid;
(R,S)-3-(4-{1-[6-(4-tert-Butyl-phenyl)-pyridin-3-yl]-4,4-dimethyl-pentyloxy}-benzoylamino)-propionic acid;
(R,S)-3-(4-{1-[6-(4-Isobutyl-phenyl)-pyridin-3-yl]-4,4-dimethyl-pentyloxy}-benzoylamino)-propionic acid;
3-(4-{1-[6-(4-Trifluoromethoxy-phenyl)-pyridin-3-yl]-hexyloxy}-benzoylamino)-propionic acid;
3-(4-{1-[6-(4-tert-Butyl-phenyl)-pyridin-3-yl]-4,4-dimethyl-pentyloxy}-benzoylamino)-propionic acid;
(R,S)-3-(4-{1-[2-(4-Trifluoromethyl-phenyl)-pyrimidin-5-yl]-heptyloxy}-benzoylamino)-propionic acid;
(R,S)-3-(4-{4,4,4-Trifluoro-1-[2-(4-trifluoromethyl-phenyl)-pyrimidin-5-yl]-butoxy}-benzoylamino)-propionic acid;
3-(4-{1-[2-(4-Trifluoromethyl-phenyl)-pyrimidin-5-yf]-heptyloxy}-benzoylamino)-propionic acid;
3-(4-{4,4,4-Trifluoro-1-[2-(4-trifluoromethyl-phenyl)-pyrimidin-5-yl]-butoxy}-benzoylamino)-propionic acid;
3-{4-[2-Cyclohexyl-1-(4′-trifluoromethyl-biphenyl-4-yl)-ethoxy]-benzoylamino}-propionic acid;
(±)-3-{4-[2-Cyclohexyl-1-(4′-trifluoromethyl-biphenyl-4-yl)-ethoxy]-benzoylamino}-propionic acid;
(R,S)-3-[4-(4′-Trifluoromethyl-biphenyl-4-ylmethoxy)-benzoylamino]-propionic acid;
(R,S)-3-{3-Allyl-4-[1-(4′-trifluoromethyl-biphenyl-4-yl)-propoxy]-benzoylamino}-propionic acid;
(R,S)-3-{3-tert-Butyl-4-[1-(4′-trifluoromethyl-biphenyl-4-yl)-propoxy]-benzoylamino}-propionic acid;
(±)-3-({4-[2-Cyclohexyl-1-(4′-trifluoromethyl-biphenyl-4-yl)-ethoxy]-benzoyl}-methyl-amino)-propionic acid;
(R,S)-3-{4-[3-Ethyl-1-(4′-trifluoromethyl-biphenyl-4-yl)-hexyloxy]-benzoylamino}-2-hydroxy-propionic acid;
2-(S)-Hydroxy-3-{4-[1-(4′-trifluoromethyl-biphenyl-4-yl)-propoxy]-benzoylamino}-propionic acid;
2-(R)-Hydroxy-3-{4-[1-(4′-trifluoromethyl-biphenyl-4-yl)-propoxy]-benzoylamino}-propionic acid;
(R,S)-3-{4-[1-(3-Methyl-4′-trifluoromethylbiphenyl-4-yl)ethoxy]benzoyl-amino}propionic acid;
(R,S)-3-{4-[1-(3-Methyl-4′-trifluoromethylbiphenyl-4-yl)butoxy]benzoyl-amino}propionic acid;
(R,S)-3-{4-[2-Methyl-1-(3-methyl-4′-trifluoromethylbiphenyl-4-yl)propoxy]benzoylamino}-propionic acid;
(R,S)-3-{4-[3-Methyl-1-(3-methyl-4′-trifluoromethylbiphenyl-4-yl)butoxy]benzoyl-amino}-propionic acid;
(R,S)-3-{4-[2-Cyclohexyl-1-(2′-trifluoromethylbiphenyl-4-yl)ethoxy]benzoyl-amino}propionic acid;
(R,S)-3-{4-[2-Cyclohexyl-1-(3′-trifluoromethylbiphenyl-4-yl)ethoxy]benzoyl-amino}propionic acid;
(R,S)-3-{4-[1-(4′-Trifluoromethylbiphenyl-4-yl)ethoxy]benzoylamino}propionic acid;
(R,S)-3-{4-[1-(4′-Trifluoromethylbiphenyl-4-yl)-propoxy]benzoylamino}propionic acid;
(R,S)-3-{4-[2-Methyl-1-(4′-trifluoromethylbiphenyl-4-yl)propoxy]benzoylamino}propionic acid;
(R,S)-3-{4-[3-Methyl-1-(4′-trifluoromethylbiphenyl-4-yl)butoxy]benzoyl-amino}propionic acid;
(R,S)-3-{4-[1-(4′-Trifluoromethylbiphenyl-4-yl)butoxy]benzoylamino}propionic acid;
(R,S)-3-{4-[1-(4′-Trifluoromethylbiphenyl-4-yl)pentyloxy]benzoylamino}propionic acid;
(R,S)-3-{4-[1-(4′-Trifluoromethylbiphenyl-4-yl)hexyloxy]benzoylamino}propionic acid;
(R,S)-3-{4-[1-(4′-Trifluoromethybiphenyl-4-yl)heptyloxy]benzoylamino}propionic acid;
(R,S)-3-{4-[1-(4′-Trifluoromethylbiphenyl-4-yl)undecyloxy]benzoylamino}propionic acid;
3-{4-[Bis-(4′-trifluoromethylbiphenyl-4-yl)methoxy]benzoylamino}propionic acid;
(R,S)-3-{4-[Phenyl-(4′-trifluoromethylbiphenyl-4-yl)methoxy]benzoylamino}propionic acid;
3-{4-[1-(4′-Trifluoromethylbiphenyl-4-yl)propoxy]benzoylamino}propionic acid, Isomer 1;
3-{4-[1-(4′-Trifluoromethylbiphenyl-4-yl)propoxy]benzoylamino}propionic acid, Isomer 2;
(R,S)-3-[4-(1-Biphenyl-4-yl-propoxy)benzoylamino]propionic acid;
(R,S)-3-{4-[1-(4′-Trifluoromethoxybiphenyl-4-yl)propoxy]benzoylamino}propionic acid;
(R,S)-3-{4-[1-(4′-Fluorobiphenyl-4-yl)propoxy]benzoylamino}propionic acid;
(R, S)-3-{4-[1-(2′,4′-Difluorobiphenyl-4-yl)propoxy]benzoylamino}propionic acid;
(R,S)-3-{4-[1-(2′,4′-Bistrifluoromethylbiphenyl-4-yl)propoxy]benzoylamino}-propionic acid;
(R,S)-3-(Methyl-{4-[1-(4′-trifluoromethylbiphenyl-4-yl)butoxy]benzoyl}amino-propionic acid;
(R,S)-3-{4-[1-(4′-Trifluoromethyl-biphenyl-4-yl)-heptyl]-benzoylamino}-propionic acid;
3-{4-[1-(4′-Trifluoromethyl-biphenyl-4-ylmethyl)-propoxy]-benzoylamino}-propionic acid;
(R,S)-3-(4-{1-[6-(4-Trifluoromethyl-phenyl)-pyridin-3-ylmethyl]-propoxy}-benzoylamino)-propionic acid;
(R,S)-3-(4-{2-[6-(4-Trifluoromethyl-phenyl)-pyridin-3-yl]-octyloxy}-benzoylamino)-propionic acid;
(R,S)-3-(4-{5,5,5-Trifluoro-2-[6-(4-trifluoromethyl-phenyl)-pyridin-3-yl]-pentyloxy}-benzoylamino)-propionic acid;
(R,S)-3-{4-[2-Cyclohexyl-1-(4′-trifluoromethyl-biphenyl-2-yl)-ethoxy]-benzoylamino}-propionic acid;
(R,S)-3-(4-{1-[4-(5-Trifluoromethyl-pyridin-2-yl)-phenyl]-pentyloxy}-benzoylamino)-propionic acid;
3-{4-[4-(5-Trifluoromethyl-pyridin-2-yl)-benzyloxy]-benzoylamino}-propionic acid;
(R,S)-3-(4-{1-[4-(5-Trifluoromethyl-pyridin-2-yl)-phenyl]-butoxy}-benzoylamino)-propionic acid;
(R,S)-3-(4- {4,4,4-Trifluoro-1- [4-(5-trifluoromethyl-pyridin-2-yl)-phenyl]}-butoxy-benzoylamino)-propionic acid;
3-(4-{1-[4-(5-Trifluoromethyl-pyridin-2-yl)-phenyl]-pentyloxy}-benzoylamino)-propionic acid, 3-(4-{4,4,4-Trifluoro-1-[4-(5-trifluoromethyl-pyridin-2-yl)-phenyl]-butoxy}-benzoylamino)-propionic acid;
3-(4-{1-[4-(5-Trifluoromethyl-pyridin-2-yl)-phenyl]-butoxy}-benzoylamino)-propionic acid;
(R,S)-Cis-3-(4-{6,6,6-Trifluoro-3-[6-(4-trifluoromethyl-phenyl)-pyridin-3-yl]-hex-1-enyl}-benzoylamino)-propionic acid;
(R,S)-Trans-3-(4-{6,6,6-Trifluoro-3-[6-(4-trifluoromethyl-phenyl)-pyridin-3-yl]-hex-1-enyl}-benzoylamino)-propionic acid;
3-(4-{4,4-Dimethyl-1-[6-(4-trifluoromethyl-phenyl)-pyridin-3-yl]-pentyloxy}-benzoylamino)-butyric acid;
3-(4-{4,4-Dimethyl-1-[6-(4-trifluoromethyl-phenyl)-pyridin-3-yl]-pentyloxy}-benzoylamino)-butyric acid;
(R,S)-3-{4-[2-(4′-Trifluoromethylbiphenyl-4-yl)butoxy]benzoylamino}propionic acid;
(R,S)-3-(4-{1-[5-(3,5-Bis-trifluoromethyl-phenyl)-pyridin-3-yl]-heptyloxy}-benzoylamino)-propionic acid;
(R,S)-3-(4-{1-[5-(4-Isobutyl-phenyl)-pyridin-3-yl]-heptyloxy}-benzoylamino)-propionic acid;
(R,S)-3-(4-{1-[5-(4-tert-Butyl-phenyl)-pyridin-3-yl]-heptyloxy}-benzoylamino)-propionic acid;
(R,S)-3-(4-{1-[5-(4-Ethyl-phenyl)-pyridin-3-yl]-heptyloxy}-benzoylamino)-propionic acid;
(R,S)-3-{4-[2-Cyclohexyl-1-(4′-trifluoromethyl-biphenyl-3-yl)-ethoxy]-benzoylamino}-propionic acid;
(R,S)-3-{4-[2-Cyclohexyl-1-(3-methyl-4′-trifluoromethyl-biphenyl-4-yl)-ethoxy]-benzoylamino}-propionic acid;
(R,S)-3-(4-{1-[4-(2,3,5,6-Tetramethyl-benzyloxy)-phenyl]-propoxy}-benzoylamino)-propionic acid;
(R,S)-{4-[1-(4-Pentamethylphenylmethoxy-phenyl)-propoxy]-benzoylamino}-propionic acid;
3-(4-{1-[4-(2,3,5,6-Tetramethyl-benzyloxy)-phenyl]-propoxy}-benzoylamino)-propionic acid;
3-(4-{1-[4-(2,3,5,6-Tetramethyl-benzyloxy)-phenyl]-propoxy}-benzoylamino)-propionic acid, (R,S)-3-(4-{1-[6-(4-Trifluoromethyl-phenoxy)-pyridin-3-yl]-heptyloxy}-benzoylamino)-propionic acid;
(R,S)-3-{4-[1-(4-Trifluoromethyl-3,4,5,6-tetrahydro-2H-[1,2]bipyridinyl-5′-yl)-heptyloxy]-benzoylamino}-propionic acid;
(R,S)-3-{4-[1-(2,6-Dimethyl-4′-trifluoromethyl-biphenyl-4-yl)-3-methyl-butoxy]-benzoylamino}-propionic acid;
(R,S)-3-{4-[1-Hydroxy-1-(4′-trifluoromethyl-biphenyl-4-yl)-heptyl]-benzoylamino}-propionic acid;
3-{4-[1-(4′-Trifluoromethyl-biphenyl-4-yl)-hept-1-enyl]-benzoylamino}-propionic acid;
(R,S)-3-{4-[1-(6-Methoxy-4′-trifluoromethyl-biphenyl-3-yl)-heptyl]-benzoylamino}-propionic acid;
3-{4-[1-(2,6-Dimethyl-4′-trifluoromethyl-biphenyl-4-yl)-3-methyl-butoxy]-benzoylamino}-propionic acid;
3-{4-[1(6-Methoxy-4′-trifluoromethyl-biphenyl-3-yl)-heptyl]-benzoylamino}-propionic acid;
(R,S)-3-(3-Fluoro-4-{1-[6-(4-trifluoromethyl-phenyl)-pyridin-3-yl]-heptyloxy}-benzoylamino)-propionic acid;
(R,S)-3-{3-Fluoro-4-[1-(4′-trifluoromethyl-biphenyl-4-yl)-heptyloxy]-benzoylamino}-propionic acid;
(R,S)-3-{3-Fluoro-4-[1-(2-methyl-4′-trifluoromethyl-biphenyl-4-yl)-heptyloxy]-benzoylamino}-propionic acid;
(R,S)-3-{4-[1-(4′-Isopropyl-2-methyl-biphenyl-4-yl)-5-methyl-hexyloxy]-benzoylamino}-propionic acid;
(R,S)-3-{4-[1-(4′-tert-Butyl-2-methyl-biphenyl-4-yl)-5-methyl-hexyloxy]-benzoylamino}-propionic acid;
(R,S)-3-{4-[1-(4′-tert-Butyl-2-methyl-biphenyl-4-yl)-heptyloxy]-benzoylamino}-propionic acid;
(R,S)-3-{4-[(4′-tert-Butyl-2-methyl-biphenyl-4-yl)-cyclohexyl-methoxy]-benzoylamino}-propionic acid;
3-{4-[(4′-tert-Butyl-2-methyl-biphenyl-4-yl)-cyclohexyl-methoxy]-benzoylamino}-propionic acid;
3-{4-[1-(4′-tert-Butyl-2-methyl-biphenyl-4-yl)-heptyloxy]-benzoylamino}-propionic acid;
3-{4-[5-Methyl-1-(2-methyl-4′-trifluoromethyl-biphenyl-4-yl)-hexyloxy]-benzoylamino}-propionic acid;
3-{4-[1-(4′-Isopropyl-2-methyl-biphenyl-4-yl)-5-methyl-hexyloxy]-benzoylamino}-propionic acid;
3-{4-[1-(4′-tert-Butyl-2-methyl-biphenyl-4-yl)-5-methyl-hexyloxy]-benzoylamino}-propionic acid;
(R,S)-3-{3-Fluoro-4-[1-(4′-trifluoromethyl-biphenyl-4-yl)-heptyloxy]-benzoylamino}-propionic acid;
(R,S)-3-{3-Fluoro-4-[1-(2-methyl-4′-trifluoromethyl-biphenyl-4-yl)-heptyloxy]-benzoylamino}-propionic acid;
(R)-3-(6-(3-methyl-1-(4′-(trifluoromethyl)biphenyl-4-yl)butylamino)nicotinamido)propanoic acid;
(+/−)-3-(6-(3-methyl-1-(4′-(trifluoromethyl)biphenyl-4-yl)butylamino)nicotinamido)propanoic acid;
(−)-(S)-3-(6-(3-methyl-1-(4′-(trifluoromethyl)biphenyl-4-yl)butylamino)nicotinamido)propanoic acid;
(+)-(R)-3-(6)-(3-methyl-1-(4′-(trifluoromethyl)biphenyl-4-yl)butylamino)nicotinamido)propanoic acid;
(+/−)-3-(2-(3-methyl-1-(4′-(trifluoromethyl)biphenyl-4-yl)butylamino)pyrimidine-5-carboxamido)propanoic acid;
3-(2-(3-methyl-1-(4′-(trifluoromethyl)biphenyl-4-yl)butylamino)pyrimidine-5-carboxamido)propanoic acid;
(+/−)-3-(6-(1(4′-(trifluoromethyl)biphenyl-4-yl)butylamino)nicotinamido)propanoic acid;
3-(6-(1-(4′-(trifluoromethyl)biphenyl-4-yl)butylamino)nicotinamido)propanoic acid;
(+/−)-3-(6-(2-methyl-1-(4′-(trifluoromethyl)biphenyl-4-yl)propylamino)nicotinamido)propanoic acid;
(+/−)-3-(6-(1(4′-(trifluoromethyl)biphenyl-4-4Propylamino)nicotinamido)propanoic acid;
3-(6-(1-(4′-(trifluoromethyl)biphenyl-4-yl)propylamino)nicotinamido)propanoic acid;
(+)-3-(6-(2-methyl-1-(4′-(trifluoromethyl)biphenyl-4-yl)propylamino)nicotinamido)propanoic acid;
(−)-3-(6-(2-methyl-1-(4′-(trifluoromethyl)biphenyl-4-yl)propylamino)nicotinamido)propanoic acid;
(+/−)-3-(5-(3-methyl-1-(4′-(trifluoromethyl)biphenyl-4-yl)butylamino)pyrazine-2-carboxamido)propanoic acid;
3-(5-(3-methyl-1-(4′-(trifluoromethyl)biphenyl-4-yl)butylamino)pyrazine-2-carboxamido)propanoic acid;
(+/−)-2-(3-methyl-1-(4′-(trifluoromethyl)biphenyl-4-yl)butylamino)-N-(1H-tetrazol-5-yl)pyrimidine-5-carboxamide;
(+/−)-N-(3-(1H-tetrazol-5-ylamino)-3-oxopropyl)-6-(3-methyl-1-(4′-(trifluoromethyl)biphenyl-4-yl)butylamino)nicotinamide;
(+/−)-N-((2H-tetrazol-5-yl)methyl)-6-(3-methyl-1-(4′-(trifluoromethyl)biphenyl-4-yl)butylamino)nicotinamide;
(+/−)-2-(6-(3-methyl-1-(4′-(trifluoromethyl) biphenyl-4-yl)butylamino)nicotinamido)ethanesulfonic acid;
(+/−)-3-(N-methyl-6-(3-methyl-1-(4′-(trifluoromethyl)biphenyl-4-yl)butylamino)nicotinamido)propanoic acid;
(+/−)-(2R)-2-hydroxy-3-(6-(3-methyl-1-(4′-(trifluoromethyl)biphenyl-4-yl)butylamino)nicotinamido)propanoic acid;
(+/−)-3-(6-(3-methyl-1-(2-methyl-4′-(trifluoromethyl)biphenyl-4-yl)butylamino)nicotinamido)propanoic acid;
(+/−)-3-(2-(3-methyl-1-(2-methyl-4′-(trifluoromethyl)biphenyl-4-yl)butylamino)pyrimidine-5-carboxamido)propanoic acid;
(+/−)-3-(2-(1-(2,6-dimethyl-4′-(trifluoromethyl)biphenyl-4-yl)-3-methylbutylamino)pyrimidine-5-carboxamido)propanoic acid;
(+/−)-3-(6-(1-(2,6-dimethyl-4′-(trifluoromethyl)biphenyl-4-yl)-3-methylbutylamino)nicotinamido)propanoic acid;
(+/−)-3-(6-(3-methyl-1-(6-(4-(trifluoromethyl)phenyl)pyridin-3-yl)butylamino)nicotinamido)propanoic acid;
(+/−)-3-(5-(1-(4′-(trifluoromethyl)biphenyl-4-yl)butylamino)pyrazine-2-carboxamido)propanoic acid;
(+/−)-3-(5-(1-(4′-(trifluoromethyl)biphenyl-4-yl)propylamino)pyrazine-2-carboxamido)propanoic acid;
(+/−)-3-(6-(cyclobutyl(4′-(trifluoromethyl)biphenyl-4-yl)methylamino) nicotinamido)propanoic acid;
(+/−)-3-(6-(3,3-dimethyl-1-(4′-(trifluoromethyl)biphenyl-4-yl)butylamino)nicotinamido)propanoic acid;
(+/−)-3-(6-(cyclopropyl(4′-(trifluoromethyl)biphenyl-4-yl)methylamino)nicotinamido)propanoic acid;
(+/−)-3-(6-(cyclopentyl(4′-(trifluoromethyl)biphenyl-4-yl)methylamino)nicotinamido)propanoic acid;
(+/−)-3-(6-(3-methyl-1-(4-(6-methylpyridin-3-yl)phenyl)butylamino) nicotinamido)propanoic acid;
(+/−)-3-(641-(biphenyl-4-yl)-3-methylbutylamino)nicotinamido)propanoic acid;
(+/−)-3-(6-(1-(2′-fluorobiphenyl-4-yl)-3-methylbutylamino) nicotinamido)propanoic acid;
(+/−)-3-(6-(1-(3′-chlorobiphenyl-4-yl)-3-methylbutylamino) nicotinamido)propanoic acid;
(+/−)-3-(6-(3-methyl-1-(4-(pyridin-3-yl)phenyl)butylamino)nicotinamido)propanoic acid;
(+/−)-3-(6-(1-(3′-fluorobiphenyl-4-yl)-3-methylbutylamino) nicotinamido)propanoic acid;
(+/−)-3-(6-(3-methyl-1-(2′-methylbiphenyl-4-yl)butylamino)nicotinamido)propanoic acid;
(+/−)-3-(6-(1-(2′-methoxybiphenyl-4-yl)-3-methylbutylamino)nicotinamido)propanoic acid; p1 (+/−)-3-(6-(1-(2′-chlorobiphenyl-4-yl)-3-methylbutylamino)nicotinamido)propanoic acid;
(+/−)-3-(6-(1-(4′-cyanobiphenyl-4-yl)-3-methylbutylamino)nicotinamido)propanoic acid;
(+/−)-3-(6-(1-(4′-ethoxybiphenyl-4-yl)-3-methylbutylamino)nicotinamido)propanoic acid;
(+/−)-3-(6-(1-(3′-methoxybiphenyl-4-yl)-3-methylbutylamino)nicotinamido)propanoic acid;
(+/−)-3-(6-(1-(2′,6′-dimethylbiphenyl-4-yl)-3-methylbutylamino)nicotinamido)propanoic acid;
(+/−)-3-(6-(1-(2′,5′-dimethylbiphenyl-4-yl)-3-methylbutylamino)nicotinamido)propanoic acid;
(+/−)-3-(6-(3-methyl-1-(4′-methylbiphenyl-4-yl)butylamino)nicotinamido)propanoic acid;
(+/−)-3-(6-(1-(4′-fluorobiphenyl-4-yl)-3-methylbutylamino)nicotinamido)propanoic acid;
(+/−)-3-(6-(1-(4′-methoxybiphenyl-4-yl)-3-methylbutylamino)nicotinamido)propanoic acid;
(+/−)-3-(6-(1-(4′-chlorobiphenyl-4-yl)-3-methylbutylamino)nicotinamido)propanoic acid;
(+/−)-3-(6-(1-(4′-ethylbiphenyl-4-yl)-3-methylbutylamino)nicotinamido)propanoic acid;
(+/−)-3-(6-(3-methyl-1-(4-(68yridine-2-yl)phenyl)butylamino)nicotinamido)propanoic acid;
(+/−)-3-(6-(1-(4′-(dimethylcarbamoyl)biphenyl-4-yl)-3-methylbutylamino)nicotinamido)propanoic acid;
(+/−)-3-(6-(1-(4′-isopropylbiphenyl-4-yl)-3-methylbutylamino)nicotinamido)propanoic acid;
(+/−)-3-(6-(3-methyl-1-(4′-(trifluoromethoxy)biphenyl-4-yl)butylamino)nicotinamido)propanoic acid;
(+/−)-3-(6-(3-methyl-1-(4′-(methylsulfonyl)biphenyl-4-yl)butylamino)nicotinamido)propanoic acid;
(+/−)-3-(6-(3-methyl-1-(4-(6-(trifluoromethyl)pyridin-3-yl)phenyl)butylamino)nicotinamido)propanoic acid;
(+/−)-3-(6-(1-(2,6-dimethyl-4′-(trifluoromethyl)biphenyl-4-ylamino)-3-methylbutyl)nicotinamido)propanoic acid;
3-(6-(1-(2,6-dimethyl-4′-(trifluoromethyl)biphenyl-4-ylamino)-3-methylbutyl)nicotinamido)propanoic acid;
(+/−)-3-(6-(3-methyl-1-(4′-(trifluoromethyl)biphenyl-4-ylamino)butyl)nicotinamido)propanoic acid;
3-(6-(3-methyl-1-(4′-(trifluoromethyl)biphenyl-4-ylamino)butyl)nicotinamido)propanoic acid; (+/−)-3-(6-(3-methyl-1-(6-(4-(trifluoromethyl)phenyl)pyridin-3-ylamino)butyl)nicotinamido)propanoic acid;
(+/−)-3-(6-(3-methyl-1-(5 -methyl-6-(4-(trifluoromethyl)phenyl)pyridin-3-ylamino)butyl)nicotinamido)propanoic acid;
(+/−)-3-(6-(1-(5-methyl-6-(4-(trifluoromethyl)phenyl)pyridin-3-ylamino)butyl)nicotinamido)propanoic acid;
(+/−)-3-(6-(1-(4′-(trifluoromethyl)biphenyl-4-ylamino)ethyl)nicotinamido)propanoic acid;
(+/−)-3-(6-(1-(5-methyl-6-(4-(trifluoromethyl)phenyl)pyridin-3-ylamino)propyl)nicotinamido)propanoic acid;
(+/−)-N-((1H-tetrazol-5-yl)methyl)-6-(3-methyl-1-(4′-(trifluoromethyl)biphenyl-4-ylamino)butyl)nicotinamide;
(+/−)-6-(3-methyl-1(4′-(trifluoromethyl)biphenyl-4-ylamino)butyl)-N-(2H-tetrazol-5-yl)nicotinamide;
(+/−)-3-(4-(1-(6-(4-(trifluoromethyl)phenyl)pyridazin-3-ylamino)butyl)benzamido)propanoic acid;
(+/−)-3-(4-(1-(2-(4-(trifluoromethyl)phenyl)pyrimidin-5-ylamino)butyl)benzamido)propanoic acid;
3-(4-(1-(2-(4-(trifluoromethyl)phenyl)pyrimidin-5-ylamino)butyl)benzamido)propanoic acid;
(+/−)-3-(6-(3-methyl-1-(4′-(trifluoromethyl)biphenyl-4-Yloxy)butyl)nicotinamido)propanoic acid;
(+/−)-3-(6-(3-methyl-1-(4′-(trifluoromethyl)biphenyl-4-yl)butoxy)nicotinamido)propanoic acid;
(+/−)-3-(6-(1-(4′-(trifluoromethyl)biphenyl-4-yloxy)ethyl)nicotinamido)propanoic acid;
(S)-N-({6-[(3-methyl-1-{4-[5-(trifluoromethyl)pyridin-2-yl]phenyl}butyl)amino]pyridin-3-yl}carbonyl)-beta-alanine
(+/−)-N-({6-[(cyclohexyl{2-[4-(trifluoromethyl)phenyl]pyrimidin-5-yl}methyl)amino]pyridin-3-yl}carbonyl)-beta-alanine
(±)-3-(4-(1-((5-(4-(trifluoromethyl)phenyl)pyrazin-2-yl) amino)butyl)benzamido)propanoic acid;
(S)-3-(4-(1-((5-(4-(trifluoromethyl)phenyl)pyrazin-2-yl)amino)butyl)benzamido)propanoic acid; and (R)-3-(4-(1-((5-(4-(trifluoromethyl)phenyl)pyrazin-2-yl)amino)butyl)benzamido)propanoic acid;
3-(N-methyl-6-((3-methyl-1-(4′-(trifluoromethyl)-[1,1′-biphenyl]-4-yl)butyl)amino)nicotinamido)propanoic acid;
(±)-3-(6-(((tetrahydro-2H-pyran-4-yl)(4′-(trifluoromethyl)-[1,1′-biphenyl]-4-yl)methyl)amino)nicotinamido)propanoic acid;
(±)-3-(6-((1-(2-cyano-4′-(trifluoromethyl)-[1,1′-biphenyl]-4-yl)butyl)amino)nicotinamido)propanoic acid;
(+/−)-3-({5-[3-methyl-1-(4′-trifluoromethyl-biphenyl-4-yl)-butylamino]-pyrimidine-2-carbonyl}-amino)-propionic acid;
3-({5-[(R)-3-methyl-1-(4′-trifluoromethyl-biphenyl-4-yl)-butylamino]-pyrimidine-2-carbonyl}-amino)-propionic acid and 3-({5-[(S)-3-methyl-1-(4′-trifluoromethyl-biphenyl-4-yl)-butylamino]-pyrimidine-2-carbonyl}-amino)-propionic acid;
3-[(6-{(S)-3-methyl-1-[2-(4-trifluoromethyl-phenyl)-pyrimidin-5-yl]-butylamino}-pyridine-3-carbonyl)-amino]-propionic acid;
(+/−)-3-(6-(4,4,4-trifluoro-1-(4′-(trifluoromethyl)biphenyl-4-yl)butylamino)nicotinamido)propanoic acid;
3-(6-(2-cyclopropyl-1-(4′-(trifluoromethyl)biphenyl-4-yl)ethylamino)nicotinamido)propanoic acid;
3-(6-(3-methyl-1-(5-methyl-6-(4-(trifluoromethyl)phenyl)pyridin-3-yl)butylamino)nicotinamido)propanoic acid;
3-(6-(1-(6-(4-chlorophenyl)-5-methylpyridin-3-ylamino)-3-methylbutyl)nicotinamido)propanoic acid;
3-(2-(1-(4′-(trifluoromethyl)biphenyl-4-yl)butylamino)pyrimidine-5-carboxamido)propanoic acid;
3-(4-(1-(6-(4-(trifluoromethyl)phenyl)pyridin-3-ylamino)butyl)benzamido)propanoic acid;
(+/−)-3-(6-(3-methyl-1-(2-(4-(trifluoromethyl)phenyl)pyrimidin-5-yl)butylamino)nicotinamido)propanoic acid;
3-(6-(3-methyl-1-(6-(4-(trifluoromethyl)phenyl)pyridin-3-yl)butylamino)nicotinamido)propanoic acid;
(+/−)-3-(6-(3-methyl-1-(4-(5-(trifluoromethyl)pyridin-2-yl)phenyl)butylamino)nicotinamido)propanoic acid;
3-(6-(1(2-methoxy-4′-(trifluoromethyl)biphenyl-4-yl)butylamino)nicotinamido)propanoic acid;
3-(6-(1-(3-methoxy-4′-(trifluoromethyl)biphenyl-4-yl)butylamino)nicotinamido)propanoic acid;
3-(6-(1(2-(4-(trifluoromethoxy)phenyl)pyrimidin-5-yl) butylamino)nicotinamido)propanoic acid;
3-(6-(cyclopentyl(4′-fluorobiphenyl-4-yl)methylamino)nicotinamido)propanoic acid, Isomer 1;
3-(6-(cyclopentyl(4′-fluorobiphenyl-4-yl)methylamino)nicotinamido)propanoic acid, Isomer 2;
3-(6-(cyclopentyl(2-(4-(trifluoromethyl)phenyl)pyrimidin-5-yl)methylamino)nicotinamido)propanoic acid;
3-(6-((tetrahydro-2H-pyran-4-yl)(4′-(trifluoromethyl)biphenyl-4-yl)methylamino)nicotinamido)propanoic acid;
3-(4-(3-methyl-1-(2-(4-(trifluoromethyl)phenyl)pyrimidin-5-ylamino)butyl)benzamido)propanoic acid;
3-(3-(3-methyl-1-(2-(4-(trifluoromethyl)phenyl)pyrimidin-5-yl)amino)butyl)picolinamido)propanoic acid;
3-(6-((3-methoxycyclobutyl)(4′-(trifluoromethyl)biphenyl-4-yl)methylamino)nicotinamido)propanoic acid;
3-(6-((tetrahydro-2H-pyran-3-yl)(4′-(trifluoromethyl)biphenyl-4-yl)methylamino)nicotinamido)propanoic acid;
3-(6-(cyclopentyl(2-(4-(trifluoromethyl)phenyl)pyrimidin-5-yl)methylamino)nicotinamido)propanoic acid;
3-(6-(1-(2-(4-(trifluoromethyl)phenyl)pyrimidin-5-yl)butylamino)nicotinamido)propanoic acid;
(S)-3-(4-(1-(3,5-dimethyl-4-(4-(trifluoromethyl)-1H-pyrazol-1-yl)phenoxy)butyl)benzamido)propanoic acid;
(+/−)-3-(4-(1-(3-methyl-4-(4-(trifluoromethyl)-1H-pyrazol-1-yl)phenylamino)butyl)benzamido)propanoic acid;
+/−)-3-(4-(3-methyl-1-(4-(4-(trifluoromethyl)-1)-1H-imidazol-1-1-yl)phenyl)butoxy)benzamido)propanoic acid;
(+/−)-3-(6-(1-(4-(4-(trifluoromethyl)-1H-pyrazol-1-yl)phenoxy)butyl)nicotinamido)propanoic acid;
(+/−)-3-(4-(4-methyl-1-(4-(4-(trifluoromethyl)-1H-pyrazol-1-yl)phenyl)pentan-2-yl)benzamido)propanoic acid;
(+/−)-3-(4-(1-(6-(4-(trifluoromethyl)-1H-pyrazol-1-yl)pyridine-3-ylamino)butyl)benzamido)propanoic acid;
(R)-3-(4-(1-(6-(4-(trifluoromethyl)-1H-pyrazol-1-yl)pyridine-3-ylamino)butyl)benzamido)propanoic acid;
(S)-3-(4-(1-(6-(4-(trifluoromethyl)-1H-pyrazol-1-yl)pyridine-3-ylamino)butyl)benzamido)propanoic acid;
(+/−)-3-(4-(cyclopentyl(6-(4-(trifluoromethyl)-1H-imidazol-1-yl)pyridine-3-ylamino)methyl)benzamido)propanoic acid;
(+/−)-3-(4-(1-(6-(4-(trifluoromethyl)-1H-imidazol-1-yl)pyridine-3-ylamino)butyl)benzainido)propanoic acid; (R)-3-(4-(1-(6-(4-(trifluoromethyl)-1H-imidazol-1-yl)pyridine-3-ylamino)butyl)benzamido)propanoic acid;
(S)-3-(4-(1-(6-(4-(trifluoromethyl)-1H-imidazol-1-yl)pyridine-3-ylamino)butyl)benzamido)propanoic acid; (+/−)-3-(4-(1-(4-(4-(trifluoromethyl)-1H-pyrazol-1-yl)phenylamino)butyl)benzamido)propanoic acid;
(+/−)-3-(4-(1-(3,5-dimethyl-4-(4-(trifluoromethyl)-1H-pyrazol-1-yl)phenylamino)butyl)benzamido)propanoic acid;
(R)-3-(4-(1-(3,5-dimethyl-4-(4-(trifluoromethyl)-1H-pyrazol-1-yl)phenylamino)butyl)benzamido)propanoic acid;
(S)-3-(4-(1-(3,5-dimethyl-4-(4-(trifluoromethyl)-1H-pyrazoi-yl)phenylamino)butylbenzamido)propanoic acid;
(+/−)-3-(4-(1-(4(4-(trifluoromethyl)-1H-imidazol-1-yl)phenlylamino)butyl)benzamido)propanoic acid;
(R)-3-(4-(1-(4-(4-(trifluoromethyl)-1H-imidazol-1-yl)phenylamino)butyl)benzamido)propanoic acid;
(S)-3-(4-(1-(4-(4-(trifluoromethyl)-1H-imidazol-1-yl)phenylamino)butyl)benzamido)propanoic acid;
(+/−)-3-(4-(1-(4-(4-(trifluoromethyl)-1H-imidazol-1-yl)phenoxy)butyl)benzamido)propanoic acid;
(+/−)-3-(4-(1-(4-(4-(methylthio)-1H-pyrazol-1-yl)phenoxy)butyl)benzamido)propanoic acid;
(+/−)-3-(4-(1-(4-(3-tert-butyl-1H-pyrazol-1-yl)phenoxy)butyl)benzamido)propanoic acid;
(+/−)-3-(4-(1-(4-(4-chloro-methyl-1H-pyrazol-1-yl)phenoxy)butyl)benzamido)propanoic acid;
)+/−)-3-(4-(194-(4-chloro-1H-pyrazol-1yl)phenoxy)butyl)benzamido)propanoic acid;
(+/−)-3-(4-(1-(4-(4-ethyl-3-methyl-1H-pyrazol-1-yl)phenoxy)buty)benzamido)propanoic acid;
(+/−)-3-(4-(1-(4-(3,5-diethyl-1H-pyrazol-1-yl)phenoxy)butyl)benzamido)propanoic acid;
(+/−)-3-(4-(1-(4-(4-methyl-1H-pyrazol-1-yl)phenoxy)butyl)benzamido)propanoic acid;
(+/−)-3-(4-(1-(4-(3-isopropyl-1H-1-pyrazol-1-yl)phenoxy)butyl)benzamido)propanoic acid;
(+/−)-3-(4-(1-(4-(4-fluoro-1H-pyrazol-1-yl)phenoxy)butyl)benzamido)propanoaic acid;
(+/−)-3-(4-(1-(4-(3-methyl-1H-pyrazol-1-yl)phenoxy)butyl)benzamido)propanoic acid;
(+/−)-3-(4-(1-(4-(2H-1,2,3-triazol-2-yl)phenoxy)butyl)benzamido)propanoic acid;
(+/−)-3-(4-(1-(4-(3-butyl-1H-pyrazo1-1-yl)phenoxy)butyl)benzamido)propanoic acid;
(+/−)-3-(4-(1-(4-(5-ethoxy-3-methyl-1H-pyrazol-1-yl)phenoxy)butyl)benzamido)propanoic acid;
(+/−-3-(4-(1-(4-(5-methoxy-3-methyl-1H-pyrazol-1-yl)phenoxy)butyl)benzamido)propanoic acid;
(+/−)-3-(4-(1-(4-(4-butyl-1H-imidazol-1-yl)phenoxy)butyl)benzamido)propanoic acid;
(+/−)-3-(4-(1-(4-(2-cyano-3,4,5-trimethyl-1H-pyrrol-1-yl)phenoxy)butyl)benzamido)propanoic acid;
(+/−)-3-4-(1-(4-(3-cyano-2,4-dimethyl-1H-pyrrol-1-yl)phenoxy)butyl)benzamido)propanoic acid;
(+/−)-3-(4-(1-(4-(2-cyano-3-methyl-1H-pyrrol-1-yl)phenoxy)butyl)benzamido)propanoic acid;
(+/−)-3-(6-(1-(4-(3-propyl-1H-pyrazol-1-yl)phenoxy)butyl)nicotinamido)propanoic acid;
(+/−)-3-(4-(1-(4-(3,4-dimethyl-1H-pyrazo1-1-yl)phenoxy)butyl)benzamido)propanoic acid;
(+/−)-3-(4-(1-(4-(1H-pyrazol-1-yl)phenoxy)butyl)benzamido)propanoic acid;
(+/−)-3-(4-(1-(4-(1H-imidazo[1,2-b]pyrazol-1-yl)phenoxy)butyl)benzamido)propanoic acid;
(+/−)-3-(4-(1-(4-(3-ethyl-1H-pyrazol-1-yl)phenoxy)butyl)benzamido)propanoic acid;
(+/−)-3-(4-(1-(4-(4-chloro-5-methyl-1H-imidazol-1-yl)phenoxy)butyl)benzamido)propanoic acid;
(+/−)-3-(4-(1-(4-(4,5-diethyl-1H-imidazol-1-yl)phenoxy)butyl)benzamido)propanoic acid;
(+/−)-3-(4-(1-(4-(3,5-dimethyl-1H-pyrazol-1-yl)phenoxy)butyl)benzamido)propanoic acid;
(+/−)-3-(4-(1-(4-(3-methyl-1H-1,2,4-triazol-1-yl)phenoxy)butyl)benzamido)propanoic acid;
(+/−)-3-(4-(1-(4-(1H-1,2,4-triazol-1-yl)phenoxy)butyl)benizamido)propanoic acid;
(+/−)-3-(4-(1-(4-(2-butyl-14H-imidazo-1-1-yl)phenoxy)butyl)benzamido)propanoic acid;
(+/−)-3-(4-(1-(4-(4,5-dimethyl-4H-imidazol-1-yl)phenoxy)butyl)benzamido)propanoic acid;
(+/−)-3-(4-(1-(4-(1-propyl-1H-pyrazol-4-yl)phenoxy)butyl)benzamido)propanoic acid;
(+/−)-3-(4-(1-(4-(1H-pyrazol-3-yl)phenoxy)butyl)benzamido)propanoic acid;
(+/−)-3-(4-(1-(4-(3,5-dimethylisoxazol-4-yl)phenoxy)butyl)benzamido)propanoic acid;
(+/−)-3-4-(1(1-methyl-3-(trifluoromethyl)-1H-pyrazol-5-yl)phenoxy)butyl)benzamido)propanoic acid;
(+/−)-3-(4-(1-(4-(1-methyl-1H-pyrazol-4-yl)phenoxy)butyl)benzamido)propanoic acid;
(+/−)-3-(4-(1-(4-(1,5-dimethyl-1H-pyrazol-4-yl)phenoxy)butyl)benzamido)propanoic acid;
(+/−)-3-(4-(1-(4-(1H-pyrazol-4-yl)phenoxy)butyl)benzamido)propanoic acid;
(+/−)-3-(4-(1-(4-(1-methyl-1H-pyrazol-5-yl)phenoxy)butyl)benzamido)propanoic acid;
(+/−)-3-(4-(1-(4-(1,3,5-trimethyl-1H-pyrazol-4-yl)phenoxy)butyl)benzamido)propanoic acid;
(+/−)-3-(4-(1-(4-(4-(trifluoromethyl)-1H-pyrazol-1-yl)phenoxy)butyl)benzamido)propanoic acid;
(R)-3-(4-(1-(4-(4-(trifluoromethyl)-1H-pyrazol-1-yl)phenoxy)butyl)benzamido)propanoic acid;
(S)-3-(4-(1-(4-(4-(trifluoromethyl)-1H-pyrazol-1-yl)phenoxy)butyl)benzamido)propanoic acid;
(+/−)-3-(4-(1-(6-(4-phenyl-1H-pyrazol-1-yl)pyridine-3-ylamino)butyl)benzamido)propanoic acid;
(+/−)-3-(4-(1-(4-(4-fluoro-1H-pyrazol-1-yl)phenylamino)butyl)benzamido)propanoic acid;
(+/−)-3-(6-(3-methyl-1-(4-(4-(trifluoromethyl)-1H-pyrazol-1-yl)phenyl)butylamino)nicotinamido)propanoic acid;
(+/−l)-3-(4-(2-cyclopropyl-1-(4-(4-(trifluoromethyl)-1H-pyrazol-1-yl)phenoxy)ethyl)benzamido)propanoic acid;
(+/−)-3-(4-(2-cyclopentyl-1-(4-(trifluoromethyl)-1H-pyrazol-1-yl)phenoxy)methyl)benzamido)propanoic acid;
(R)-3-(4-(cyclopentyl(4-(4-(trifluoromethyl)-1H-pyrazol-1-yl)phenoxy)methyl)benzamido)propanoic acid;
(S)-3-(4-(cyclopentyl(4-(4-(trifluoromethyl)-1H-1-pyrazol-1-yl)phenoxy)methyl)benzamido)propanoic acid;
(+/−)-3-(4-(cyclobutyl(4-(4-(trifluoromethyl)-1H-pyrazol-1-yl)phenoxy)methyl)benzamido)propanoic acid;
(+/−)-3-(4-(1-(4-(3-(trifluoromethyl-1H--pyrazol-1-yl)phenoxy)butyl)benzamido)propanoic acid;
(+/−)-3-(4-(3,3-dimethyl-1-(4-(4-(trifluoromethyl)-1H-pyrazol-1-ylphenoxy)butyl)benzamido)propanoic acid;
(+/−)-3-(4-(1-(4-(4-methyl-3-(trifluoromethyl)-1H-pyrazol-1-yl)phenoxy)butyl)benzamido)propanoic acid;
(+/−)-3-(4-(1-(4-(3-(trifluoromethyl)-1H--1,2,4-triazol-1-yl)phenoxy)butyl)benzamido)propanoic acid;
(+/−)-3-(4-(1-(4-(3-methyl-4-(trifluoromethyl)-1H-pyrazol-1-yl)phenoxy)butyl)benzamido)propanoic acid;
(+/−)3-(4-(1-(4-(2-methyl-4-(trifluoromethyl)-1H-imidazol-1-yl)phenoxy)butyl)benzamido)propanoic acid;
(+/−)-3-(4-(cyclopropyl(4-(4-(trifluoromethyl)-1H-pyrazol-1-yl)phenoxy)methyl)benzamido)propanoic acid;
(+/−)-3-(4-(2-methyl-1-(4-(4-(trifluoromethyl)- 11⁻-1-pyrazol-1-yl)phenoxy)propyl)benzamido)propanoic acid;
(+/−)-3-(4-(1-(4-(4-(trifluoromethyl)-1H-pyrazol-1-yl)phenoxy)propyl)benzamido)propanoic acid;
(+/−)-3-(4-(3-methyl-1-(4-(4-(trifluoromethyl)-1H-imidazol-1-yl)phenyl)butoxy)benzamido)propanoic acid;
(+/−)-3-(4-(3-methyl-1-4-(4-(trifluoromethyl)-1H-1-pyrazol-1-yl)phenoxy)butyl)benzamido)propanoic acid;
(+/−)-3-(4-(1-(3,5-dimethyl-4-(1H-(trifluoromethyl)-1H-pyrazol-1-yl)phenoxy)butyl)benzamido)propanoic acid;
(S)-3-(4-(1-(3,5-dimethyl-4-(4-(trifluoromethyl)-1H-pyrazol-1-yl)phenoxy)butyl)benzamido)propanoic acid;
(R-3-(4-(1-(3,5-dimethyl-4-(4-(trifluoromethyl)-1H-pyrazol-1-yl)phenoxy)butyl)benzamido)propanoic acid;
(+/−)-3-(4-(1-(5-(4-(trifluoromethyl)-1H-pyrazol-1-yl)pyridine-2-yloxy)butyl)benzamido)propanoic acid;
(+/−)-3-(4-(1-(6-(4-(trifluoromethyl)-1H-pyrazol-yl)pyridine-3-yloxy)butyl)benzamido)propanoic acid;
(+/−)-3-4-(1-(6-(4-(trifluoromethyl)-1H-imidazol-1-yl)pyridine-3-yloxy)butyl)benzamido)propanoic acid;
(+/−)-3-(4-(1-(4-(4-cyano-1H-pyrazol-1-yl)phenoxy)butyl)benzamido)propanoic acid;
(+/−)-3-(4-(1-(4-(4,5,6,7-tetrahydro-2H-indazol-2-yl)phenoxy)butyl)benzamido)propanoic acid;
(+/−)-3-(4-(1-(4-(5,6-dihydrocyclopenta[c]pyrazol-2(4H)-yl)phenoxy)butyl)benzamido)propanoic acid;
(+/−)-3-(4-(1-(4-(2H-indazol-2-yl)phenoxy)butyl)benzamido)propanoic acid;
(+/−)-3-(4-(1-(4-(4-methyl-1H-1,2,3-triazol-1-yl)phenylamino)butyl)benzamido)propanoic acid;
(+/−)-3-(2-(3-methyl-1-(4-(4-(trifluorornethyl)-1H-pyrazol-1-yl)phenyl)butylamino)pyrimidine-5-carboxamido)propanoic acid;
(+/−)-3-(4-(cyclopentyl(6-(4-(trifluoromethyl)-1H-prazol-1-yl)pyridine-3-ylamino)methyl)benzamido)propanoic acid;
(R)-3-(4-(cyclopentyl(6-(4-(trifluoromethyl)-1H-pyrazol-1-yl)pyridine-3-ylamino)methyl)benzamido)propanoic acid;
(S)-3-(4-(cyclopentyl(6-(4-(trifluoromethyl)-1H-pyrazol-1-yl)pyridine-3-ylamino)methyl)benzamido)propanoic acid;
(R)-3-(4-(cyclopentyl(6-(4-(trifluoromethyl)-1H-imidazol-1-yl)pyridin-3-ylamino)methyl)benzamido)propanoic acid;
(S)-3-(4-(cyclopentyl(6-(4-(trifluoromethyl)-1H-imidazol-1-yl)pyridin-3-ylamino)methyl)benzamido)propanoic acid;
(+/−)-3-(2-(cyclohexyl(6-(4-(trifluoromethy)-1H-imidazol-1-yl)pyridine-3-yl)methylamino)benzamido)propanoic acid;
(+/−)-3-(4-(3,3-dimethyl-1-(6-(4-(trifluoromethyl-1H-imidazol-1-yl)pyridine-3-ylamino)butyl)benzamido)propanoic acid;
(+/−)-3-(4-(cyclohexyl(6-(4-(trifluoromethyl)-1H-pyrazol-1-yl)pyridine-3-ylamino)methyl)benzamido)propanoic acid;
(+/−)-3-(6-(3-methyl-1-(5-methyl-6-(4-(trifluoromethyl)-1H-pyrazol-1-yl)pyridin-3-ylamino)butyl)nicotinamido)propanoic acid;
(R)-3-(4-(1-(4-(4-(trifluoromethyl)-1H-pyrazol-1-yl)phenylamino)butyl)benzamido)propanoic acid;
(S)-3-(4-(1-(4-(4-(4-(trifluoromethyl)-1H-pyrazol-1-ylphenylamino)butyl)benzamido)propanoic acid;
(+/−)-3-(4-(1-(3-methylquinolin-2-ylamino)butyl)benzamido) propanoic acid;
(+/−)-3-{4-[3-methyl-1-(quinolin-3-ylamino)-butyl]-benzoylamino}-propionic acid;
(+/−)-3-{4-[1-(7-fluoro-quinazolin-2-ylamino)-3-tnethyl-butyl]-benzoylamino}-propionic acid;
(+/−)-3-{4-[3-methyl-1-(quinolin-2-ylamino)-butyl]-benzoylamino}-propionic acid;
(+/−)-3-{4-[1-(8-methoxy-quinolin-2-ylamino)-3-methyl-butyl]-benzoylamino}-propionic acid;
(+/−)-3-{4-[3-methyl-1-(3-methyl-quinoxalin-2-ylamino)-butyl]-benzoylamino}-propionic acid;
(+/−)-3-{4-[3-methyl-1-(quinoxalin-2-ylamino)-butyl]-benzoylamino}-propionic acid;
(+/−)-3-{4-[3-methyl-1-(4-methyl-quinolin-2-ylamino)-butyl]-benzoylamino}-propionic acid;
(+/−)-3-{4-[3-methyl-1-(3-methyl-quinolin-2-ylamino)-butyl]-benzoylamino}-propionic acid;
(+/−)-3-{4-[1-(7-fluoro-4-methyl-quinolin-2-ylamino)-3-methyl-butyl]-benzoylamino}-propionic acid;
(+/−)-3{-4-[1-(8-chloro-quinolin-2-ylamino)-3-methyl-butyl]-benzoylamino}-propionic acid;
(+/−)-3-{4-[3-methyl-1-(quinazolin-2-ylamino)-butyl]-benzoylamino}-propionic acid;
(+/−)3-(4-(3-methyl-1-(7-(trifluoromethyl)quinolin-2-ylamino)butyl)benzamido)propanoic acid;
(+/−)-3-(4-(3-methyl-1-(6-(trifluoromethyl)quinolin-2-ylamino)butyl)benzamido)propanoic acid;
(+\−)-3-(4-(3-methyl-1-(2-methylquinolin-3-ylamino)butyl)benzamido)propanoic acid;
(+\−)-3-(4-(3-methyl-1-(4-methylquinolin-3-ylamino)butyl)benzamido)propanoic acid;
(+/−)-3-{4-[(3,3-dimethyl-cyclobutyl)-(3-methyl-quinolin-2-ylamino)-methyl]-benzoylamino}-propionic acid;
(+/−)-3-{4-[(3,3-dimethyl-cyclobutyl)-(6-fluoro-3 methyl-quinolin-2-ylamino)-methyl]-benzoylamino}-propionic acid;
(+/−)-3-{4-[(3,3-dimethyl-cyclobutyl)-(7-fluoro-3-methyl-quinolin-2-ylamino)-methyl]-benzoylamino}-propionic acid;
(+/−3-(4-((3,3-dimethylcyclobutyl)(6-fluoroquinolin-3-ylamino)methyl)benzamido)propanoic acid;
(+/−)-3-(4-((3,3-dimethylcyclobutyl)(7-fluoroquinolin-3-ylamino)methyl)benzamido)propanoic acid;
(+/−)-3-(4-(4,4,4-trifluoro-1-(quinolin-3-ylamino)butyl)benzamido)propanoic acid;
(+/+)-3-(4-((6,7-difluoroquinolin-3-ylamino)(3,3-dimethylcyclobutyl)methyl)benzamido)propanoic acid;
(+/−)-3-(4-(3-methyl-1-(7-methylquinolin-3-ylamino)butyl)benzamido)propanoic acid;
(+/−)-3-(4-(3-methyl-1-(8-methylquinolin-3-ylamino)butyl)benzamido)propanoic acid;
(+/−)-3-(4-(3-methyl-1-(6-methylquinolin-3-ylamino)butyl)benzamido)propanoic acid;
(+/−)-3-(4-(3-methyl-1-(5-meyhylquinolin-3-ylamino)butyl)benzamido)propanoic acid; and

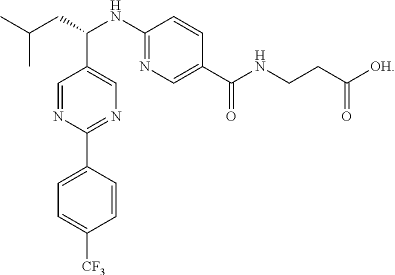 [SMILES: CC(C)C[C@H](Nc1ccc(C(=O)NCCC(=O)O)cn1)c1cnc(-c2ccc(C(F)(F)F)cc2)nc1]

### Glucagon Receptor and Antigen Binding and Antagonizing Proteins

Glucagon is a 29 amino acid hormone processed from its pre-pro-form in the pancreatic alpha cells by cell specific expression of prohormone convertase 2 (PC2), a neuroendocrine-specific protease involved in the intracellular maturation of prohormones and proneuropeptides (Furuta et al., J. Biol. Chem. 276: 27197-27202 (2001)). In vivo, glucagon is a major counter-regulatory hormone for insulin actions. During fasting, glucagon secretion increases in response to falling glucose levels. Increased glucagon secretion stimulates glucose production by promoting hepatic glycogenolysis and gluconeogenesis (Dunning and Gerich, Endocrine Reviews, 28:253-283 (2007)). Thus glucagon counterbalances the effects of insulin in maintaining normal levels of glucose in animals.

The biological effects of glucagon are mediated through the binding and subsequent activation of a specific cell surface receptor, the glucagon receptor. The glucagon receptor (GCGR) is a member of the secretin subfamily (family B) of G-protein-coupled receptors. The human GCGR is a 477 amino acid sequence GPCR and the amino acid sequence of GCGR is highly conserved across species (Mayo et al, Pharmacological Rev., 55:167-194, (2003)). The glucagon receptor is predominantly expressed in the liver, where it regulates hepatic glucose output, on the kidney, and on islet β-cells, reflecting its role in gluconeogenesis. The activation of the glucagon receptors in the liver stimulates the activity of adenyl cyclase and phosphoinositol turnover which subsequently results in increased expression of gluconeogenic enzymes including phosphoenolpyruvate carboxykinase (PEPCK), fructose-1,6-bisphosphatase (FBPase-1), and glucose-6-phosphatase (G-6-Pase). In addition, glucagon signaling activates glycogen phosphorylase and inhibits glycogen synthase. Studies have shown that higher basal glucagon levels and lack of suppression of postprandial glucagon secretion contribute to diabetic conditions in humans (Muller et al., N Eng J Med 283: 109-115 (1970)). As such, methods of controlling and lowering blood glucose by targeting glucagon production or function using a GCGR antagonist have been explored.

A further embodiment of the invention includes a process of treating heart failure comprising:

A] prescribing

B] selling or advertising to sell,

C] purchasing,

D] instructing to self-administer, or

E] administering

of a compound described herein, wherein the compound has been approved by a regulatory agency for the treatment of heart failure, to a subject in need of treatment.

A further embodiment of the invention includes a method of supplying a glucagon receptor antagonist for treating heart failure, said method comprises reimbursing a physician, a formulary, a patient or an insurance company for the sale of said glucagon receptor.

For clarity, the term “instructing” is meant to include information on a label approved by a regulatory agency, in addition to its commonly understood definition.

### Pharmaceutical Compositions

The dose, prophylactic or therapeutic, of the glucagon receptor antagonist compounds will, of course, vary with the nature or severity of the condition to be treated, the particular compound selected and its route of administration. It may also vary according to the age, weight and response of the individual patient. In general, the daily dose range for the individual compounds lies within the range of from about 0.001 mg to about 100 mg per kg, in additional embodiments about 0.01 mg to about 50 mg per kg, and in further embodiments 0.1 to 10 mg per kg, in single or divided doses. It may be necessary to use dosages outside of these limits in some cases. A therapeutically effective amount or a therapeutic or prophylactic dose or terms of similar meaning appearing throughout the application addressing the amount of the compound to be used refer to the dosage ranges provided, taking into account any necessary variation outside of these ranges. These amounts can be readily determined by the skilled physician.

Representative dosages of the glucagon receptor antagonist for adults range from about 0.1 mg to about 1.0 g per day, preferably about 1 mg to about 500 mg, in single or divided doses. Examples of suitable dosages per day include 0.1 mg, 1 mg, 2 mg, 5 mg, 10 mg, 20 mg, 40 mg, 50 mg, 75 mg, 100 mg, 150 mg, 200 mg, 250 mg, 500 mg, 1000 mg and similar such doses.

In particular embodiments, the present invention relates to methods and compositions herein where the dosage of the glucagon receptor antagonist lies within the range of from about 0.001 mg to about 100 mg per kg, in additional embodiments about 0.01 mg to about 50 mg per kg, and in further embodiments 0.1 to 10 mg per kg, in single or divided doses. In particular embodiments, the dosage is in a range of 0.1 mg per kg to 5 mg per kg. In particular embodiments, the dosage of glucagon receptor antagonist is below 0.1 mg per kg. In particular embodiments, the present invention relates to methods and compositions herein where the dosage of glucagon receptor antagonist is any amount in the range of 10-120 mg per day. In specific embodiments, the dosage of glucagon receptor antagonist is 10, 20 mg, 40 mg, 60 mg, 80, 100 mg or 120 mg per day. In specific embodiments, the dosage of glucagon receptor antagonist is any amount in the range of 1-100 mg per day. In more specific embodiments, the dosage of glucagon receptor antagonist is any amount in the range of 6-50 mg per day.

In particular embodiments, the present invention relates to methods and compositions herein where (a) the dosage of glucagon receptor antagonist is (i) in a range of 0.1 mg per kg to 2 mg per kg; (ii) below 0.1 mg per kg, (iii) in the range of 10-120 mg per day, (iv) 10 mg per day, (v) 20 mg per day, (vi) 40 mg per day, (vii) 60 mg per day, (viii) 80 mg per day, (ix) 100 mg per day, (x) 120 mg per day, (xi) in the range of 1-100 mg per day and/or (xii) in the range of 6-50 mg per day.

When intravenous administration is employed, a representative dosage range is from about 0.001 mg to about 100 mg (preferably from 0.01 mg to about 10 mg) per kg of body weight per day of each of the glucagon receptor antagonist, and, in particular embodiments about 0.1 mg to about 10 mg of the compounds per kg of body weight per day.

The glucagon receptor antagonist is used with one or more pharmaceutically acceptable carriers. Pharmaceutical compositions of use herein comprise a glucagon receptor antagonist and at least one pharmaceutically acceptable carrier. The term “composition” encompasses a product comprising the active and inert ingredient(s), any pharmaceutically acceptable excipients that make up the carrier, as well as any product which results, directly or indirectly, from the combination, complexation or aggregation of any two or more of the ingredients, or from dissociation of one or more of the ingredients, or from other types of reactions or interactions between ingredients. The composition comprises therapeutically effective amounts of the glucagon receptor antagonist.

Any suitable route of administration may be employed for providing a mammal, especially a human with an effective dosage of the glucagon receptor antagonist. For example, oral, rectal, topical, parenteral, ocular, pulmonary, nasal, and the like may be employed. Examples of dosage forms include tablets, troches, dispersions, suspensions, solutions, capsules, creams, ointments, aerosols and the like, with oral tablets being preferred.

In preparing oral compositions, any of the usual pharmaceutical media may be employed, such as, for example, water, glycols, oils, alcohols, flavoring agents, preservatives, coloring agents and the like, in the case of oral liquids, e.g., suspensions, elixirs and solutions; or carriers such as starches, sugars, microcrystalline cellulose, diluents, granulating agents, lubricants, binders, disintegrating agents and the like in the case of oral solids, e.g., powders, capsules and tablets. Solid oral preparations are preferred. Because of their ease of administration, tablets and capsules represent the most advantageous oral dosage unit forms. If desired, tablets may be coated by standard aqueous or nonaqueous techniques.

In addition to the common dosage forms set out above, the glucagon receptor antagonists may also be administered by controlled release means and/or delivery devices such as those described in U. S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any acceptable pharmaceutical process. All such methods include the step of combining the active glucagon receptor antagonist with the carrier components. In general, the compositions are prepared by uniformly and intimately admixing the active ingredients with a liquid or finely divided solid carrier component, and then, if necessary, manipulating the blend into the desired product form. For example, a tablet may be prepared by compression or molding. Compressed tablets may be prepared by compressing free-flowing powder or granules, containing the actives optionally mixed with one or more excipients, e.g., binders, lubricants, diluents, surfactants and dispersants. Molded tablets may be made by molding a mixture of the powdered compound moistened with an inert liquid. Desirably, each tablet may contain, for example, from about 0.1 mg to about 1.0 g of the active ingredient and each cachet or capsule contains from about 0.1 mg to about 500 mg of the active ingredient.

Alternatively, combinations including the glucagon receptor antagonist can be administered in a separate dosage form for simultaneous or sequential administration. In specific embodiments, the period of time between administration of the combination components is in the range from 0 minutes to 12 hours. The administration, contemporaneously or sequentially, may be once, twice, three times or four times daily, per compound or combination.

A pharmaceutical composition which is present as a separate or multiple dosage form, preferably as a kit of parts, is useful in combination therapy to flexibly suit the individual therapeutic needs of the patient.

In specific embodiment, a kit of parts comprises a containment containing a dosage form comprising the glucagon receptor antagonist and at least one pharmaceutically acceptable carrier, and at least one pharmaceutically acceptable carrier.

A further aspect of the present invention is a manufacture comprising the pharmaceutical composition being present as separate dosage forms according to the present invention and a label or package insert comprising instructions that the separate dosage forms are to be administered contemporaneously or sequentially.

A yet further aspect of the present invention is a manufacture comprising a medicament which comprises a glucagon receptor antagonist according to the present invention and a label or package insert which comprises instructions that the medicament may or is to be administered contemporaneously or sequentially with a medicament comprising a second component of the present invention.

Synthesis of the compounds and development and manufacture of pharmaceutical compositions comprising same is well understood in the art.

As used herein, “pharmaceutically acceptable carrier” includes any and all solvents, dispersion media, coatings, antibacterial and antifungal agents, isotonic and absorption delaying agents, and the like that are physiologically compatible. Some examples of pharmaceutically acceptable carriers are water, saline, phosphate buffered saline, dextrose, glycerol, ethanol and the like, as well as combinations thereof. In many cases, the composition will include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the glucagon receptor modifier. Pharmaceutical compositions of the present invention and methods for their preparation will be readily apparent to those skilled in the art. Such compositions and methods for their preparation may be found, for example, in Remington's Pharmaceutical Sciences, 19th Edition (Mack Publishing Company, 1995). The pharmaceutical compositions are generally formulated as sterile, substantially isotonic and in full compliance with all GMP regulations of the U.S. Food and Drug Administration.

The pharmaceutical compositions of the present invention are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous injection, intraperitoneal injection, intramuscular injection, intrasternal injection, intravenous injection, intraarterial injection, intrathecal injection, intraventricular injection, intraurethral injection, intracranial injection, intrasynovial injection or infusions; or kidney dialytic infusion techniques.

In various embodiments, the glucagon receptor antagonist or modulator is admixed with a pharmaceutically acceptable carrier to form a pharmaceutical composition that can be systemically administered to the subject orally or via intravenous injection, intramuscular injection, subcutaneous injection, intraperitoneal injection, transdermal injection, intra-arterial injection, intrasternal injection, intrathecal injection, intraventricular injection, intraurethral injection, intracranial injection, intrasynovial injection or via infusions.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain carriers such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

For example, in one aspect, sterile injectable solutions can be prepared by incorporating a glucagon receptor antagonist in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation such as vacuum drying and freeze-drying yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin. In various embodiments, the injectable compositions will be administered using commercially available disposable injectable devices.

The antagonist of the present invention can be administered intranasally or by inhalation, typically in the form of a dry powder (either alone, as a mixture, or as a mixed component particle, for example, mixed with a suitable pharmaceutically acceptable carrier) from a dry powder inhaler, as an aerosol spray from a pressurized container, pump, spray, atomiser (preferably an atomiser using electrohydrodynamics to produce a fine mist), or nebulizer, with or without the use of a suitable propellant, or as nasal drops. The pressurized container, pump, spray, atomizer, or nebulizer generally contains a solution or suspension of an antagonist of the invention comprising, for example, a suitable agent for dispersing, solubilizing, or extending release of the active, a propellant(s) as solvent. Prior to use in a dry powder or suspension formulation, the drug product is generally micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying. Capsules, blisters and cartridges for use in an inhaler or insufflator may be formulated to contain a powder mix of the antagonist of the invention, a suitable powder base and a performance modifier. Suitable flavors, such as menthol and levomenthol, or sweeteners, such as saccharin or saccharin sodium, may be added to those formulations of the invention intended for inhaled/intranasal administration. Formulations for inhaled/intranasal administration may be formulated to be immediate and/or modified release. Modified release formulations include delayed-, sustained-, pulsed-, controlled-, targeted and programmed release. In the case of dry powder inhalers and aerosols, the dosage unit is determined by means of a valve which delivers a metered amount. Units in accordance with the invention are typically arranged to administer a metered dose or “puff” of a glucagon receptor antagonist of the invention. The overall daily dose will typically be administered in a single dose or, more usually, as divided doses throughout the day.

Pharmaceutical compositions intended for oral use may be prepared according to any method known to the art for the manufacture of pharmaceutical compositions and such compositions may contain one or more agents selected from the group consisting of sweetening agents in order to provide a pharmaceutically elegant and palatable preparation. For example, to prepare orally deliverable tablets, the glucagon receptor antagonist is mixed with at least one pharmaceutical carrier, and the solid formulation is compressed to form a tablet according to known methods, for delivery to the gastrointestinal tract. The tablet composition is typically formulated with additives, e.g. a saccharide or cellulose carrier, a binder such as starch paste or methyl cellulose, a filler, a disintegrator, or other additives typically usually used in the manufacture of medical preparations. To prepare orally deliverable capsules, DHEA is mixed with at least one pharmaceutical carrier, and the solid formulation is placed in a capsular container suitable for delivery to the gastrointestinal tract. Compositions comprising a glucagon receptor antagonist may be prepared as described generally in Remington's Pharmaceutical Sciences, 18th Ed. 1990 (Mack Publishing Co. Easton Pa. 18042) at Chapter 89, which is herein incorporated by reference.

In various embodiments, the pharmaceutical compositions are formulated as orally deliverable tablets containing a glucagon receptor antagonist in admixture with non-toxic pharmaceutically acceptable carriers which are suitable for manufacture of tablets. These carriers may be inert diluents, such as calcium carbonate, sodium carbonate, lactose, calcium phosphate or sodium phosphate; granulating and disintegrating agents, for example, maize starch, gelatin or acacia, and lubricating agents, for example, magnesium stearate, stearic acid, or talc. The tablets may be uncoated or they may be coated with known techniques to delay disintegration and absorption in the gastrointestinal track and thereby provide a sustained action over a longer period of time. For example, a time delay material such as glyceryl monostearate or glyceryl distearate alone or with a wax may be employed.

In various embodiments, the pharmaceutical compositions are formulated as hard gelatin capsules wherein the glucagon receptor antagonist is mixed with an inert solid diluent, for example, calcium carbonate, calcium phosphate, or kaolin or as soft gelatin capsules wherein the glucagon receptor antagonist is mixed with an aqueous or an oil medium, for example, arachis oil, peanut oil, liquid paraffin or olive oil.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

### Methods of Treatment

A glucagon receptor antagonist according to the present invention, need not affect a complete cure, or eradicate every symptom or manifestation of a disease, to constitute a viable therapeutic agent. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the invention is directed to a method comprising administering to a subject a glucagon receptor antagonist in an amount and for a time sufficient to induce a sustained improvement over baseline of an indicator that reflects the severity of the particular disorder.

A subject's levels of blood glucose may be monitored before, during and/or after treatment with a glucagon receptor antagonist, to detect changes, if any, in their levels. For some disorders, the incidence of elevated blood glucose may vary according to such factors as the stage of the disease. Known techniques may be employed for measuring glucose levels. Glucagon levels may also be measured in the subject's blood using known techniques, for example, ELISA.

A therapeutically effective dose can be estimated initially from cell culture assays by determining an IC₅₀. A dose can then be formulated in animal models to achieve a circulating plasma concentration range that includes the IC₅₀ as determined in cell culture. Such information can be used to more accurately determine useful doses in humans. The exact composition, route of administration and dosage can be chosen by the individual physician in view of the subject's condition.

Thus, the skilled artisan would appreciate, based upon the invention provided herein, that the dose and dosing regimen is adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present invention.

It is to be noted that dosage values may vary with the type and severity of the condition to be ameliorated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this invention may be based on a variety of factors, including the type of disease, the age, weight, sex, medical condition of the subject, the severity of the condition, the route of administration, and the particular glucagon receptor antagonist employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present invention encompasses intra-subject dose-escalation as determined by the skilled artisan. Determining appropriate dosages and regimens are well-known in the relevant art and would be understood to be encompassed by the skilled artisan once provided the teachings disclosed herein.

In various embodiments, single or multiple administrations of the pharmaceutical compositions are administered depending on the dosage and frequency as required and tolerated by the subject. In any event, the composition should provide a sufficient quantity of at least one of the glucagon receptor antagonist disclosed herein to effectively treat the subject. The dosage can be administered once but may be applied periodically until either a therapeutic result is achieved or until side effects warrant discontinuation of therapy.

The dosing frequency of the administration of glucagon receptor antagonist pharmaceutical composition depends on the nature of the therapy and the particular disease being treated. TAs used herein, the terms “co-administration”, “co-administered” and “in combination with”, referring to the glucagon receptor antagonist of the present invention and one or more other therapeutic agent(s), is intended to mean, and does refer to and include the following: simultaneous administration of such combination of glucagon receptor antagonist of the invention and therapeutic agent(s) to a subject in need of treatment, when such components are formulated together into a single dosage form which releases said components at substantially the same time to said subject; substantially simultaneous administration of such combination of a glucagon receptor antagonist of the invention and therapeutic agent(s) to a subject in need of treatment, when such components are formulated apart from each other into separate dosage forms which are taken at substantially the same time by said subject, whereupon said components are released at substantially the same time to said subject; sequential administration of such combination of glucagon receptor antagonist of the invention and therapeutic agent(s) to a subject in need of treatment, when such components are formulated apart from each other into separate dosage forms which are taken at consecutive times by said subject with a significant time interval between each administration, whereupon said components are released at substantially different times to said subject; and sequential administration of such combination of a glucagon receptor antagonist of the invention and therapeutic agent(s) to a subject in need of treatment, when such components are formulated together into a single dosage form which releases said components in a controlled manner whereupon they are concurrently, consecutively, and/or overlappingly released at the same and/or different times to said subject, where each part may be administered by either the same or a different route.

Suitable pharmaceutical agents that may be used in combination with the compounds of the present invention include antihypertensive agents and agents for treating chronic heart failure, atherosclerosis or related diseases. Such agents contemplated for use include, but are not limited to, bimoclomol, angiotensin-converting enzyme inhibitors (such as captopril, enalapril, fosinopril, lisinopril, perindopril, quinapril, ramipril and the like), neutral endopeptidase inhibitors (such as thiorphan, omapatrilat, MDL-100240, fasidotril, sampatrilat, GW-660511, mixanpril, SA-7060, E-4030, SLV-306, ecadotril and the like), angiotensin II receptor antagonists (such as candesartan cilexetil, eprosartan, irbesartan, losartan, olmesartan medoxomil, telmisartan, valsartan, tasosartan, enoltasosartan and the like), endothelin-converting enzyme inhibitors (such as CGS 35066, CGS 26303, CGS-31447, SM-19712 and the like), endothelin receptor antagonists (such as tracleer, sitaxsentan, ambrisentan, L-749805, TBC-3214, BMS-182874, BQ-610, TA-0201, SB-215355, PD-180988, BMS-193884, darusentan, TBC-3711, bosentan, tezosentan, J-104132, YM-598, S-0139, SB-234551, RPR-118031A, ATZ-1993, RO-61-1790, ABT-546, enlasentan, BMS-207940 and the like), diuretic agents (such as hydrochlorothiazide, bendroflumethiazide, trichlormethiazide, indapamide, metolazone, furosemide, bumetamide, torsemide, chlorthalidone, metolazone, cyclopenthiazide, hydroflumethiazide, tripamide, mefruside, benzylhydrochlorothiazide, penflutizide, methyclothiazide, azosemide, etacrynic acid, torasemide, piretamide, meticrane, potassium canrenoate, spironolactone, triamterene, aminophylline, cicletanine, LLU-alpha, PNU-80873A, isosorbide, D-mannitol, D-sorbitol, fructose, glycerin, acetazolamide, methazolamide, FR-179544, OPC-31260, lixivaptan, conivaptan and the like), calcium channel antagonists (such as amlodipine, bepridil, diltiazem, felodipine, isradipine, nicardipen, nimodipine, verapamil, S-verapamil, aranidipine, efonidipine, barnidipine, benidipine, manidipine, cilnidipine, nisoldipine, nitrendipine, nifedipine, nilvadipine, felodipine, pranidipine, lercanidipine, isradipine, elgodipine, azelnidipine, lacidipine, vatanidipine, lemildipine, diltiazem, clentiazem, fasudil, bepridil, gallopamil and the like), vasodilating antihypertensive agents (such as indapamide, todralazine, hydralazine, cadralazine, budralazine and the like), beta blockers (such as acebutolol, bisoprolol, esmolol, propanolol, atenolol, labetalol, carvedilol, metoprolol and the like), sympathetic blocking agents (such as amosulalol, terazosin, bunazosin, prazosin, doxazosin, propranolol, atenolol, metoprolol, carvedilol, nipradilol, celiprolol, nebivolol, betaxolol, pindolol, tertatolol, bevantolol, timolol, carteolol, bisoprolol, bopindolol, nipradilol, penbutolol, acebutolol, tilisolol, nadolol, urapidil, indoramin and the like), alpha-2-adrenoceptor agonists (such as clonidine, methyldopa, CHF-1035, guanabenz acetate, guanfacine, moxonidine, lofexidine, talipexole and the like), centrally acting antihypertensive agents (such as reserpine and the like), thrombocyte aggregation inhibitors (such as warfarin, dicumarol, phenprocoumon, acenocoumarol, anisindione, phenindione, ximelagatran and the like), antiplatelets agents (such as aspirin, clopidogrel, ticlopidine, dipyridamole, cilostazol, ethyl icosapentate, sarpogrelate, dilazep, trapidil, beraprost and the like), and neuregulins (NRG-1, NRG-2, NRG-3 and NRG-4) and isoforms thereof.

In various embodiments, the present invention comprises a method for treating or preventing heart failure and associated conditions in a subject, comprising administering to a subject diagnosed with heart failure, or a subject at risk of contracting heart failure, a therapeutically effective amount of a glucagon receptor antagonist; and (b) a second agent composition. In various embodiments, the second agent composition is selected from a group consisting of: angiotensin-converting enzyme (ACE) inhibitors, (3-adrenergic blocking agents, angiotension II receptor blockers (ARBs), diuretics, and digitalis.

In various embodiments, the combination therapy comprises administering a glucagon receptor antagonist and the second agent composition simultaneously, either in the same pharmaceutical composition or in separate pharmaceutical compositions. In various embodiments, a glucagon receptor antagonist composition and the second agent composition are administered sequentially, i.e., the glucagon receptor antagonist composition is administered either prior to or after the administration of the second agent composition.

In various embodiments, the administrations of the glucagon receptor antagonist composition and the second agent composition are concurrent, i.e., the administration period of the glucagon receptor antagonist composition and the second agent composition overlap with each other.

In various embodiments, the administrations of the glucagon receptor antagonist composition and the second agent composition are non-concurrent. For example, in various embodiments, the administration of the glucagon receptor antagonist composition is terminated before the second agent composition is administered. In various embodiments, the administration second agent composition is terminated before the glucagon receptor antagonist composition is administered.

The invention having been described, the following examples are offered by way of illustration, and not limitation.

### EXAMPLE 1

To evaluate the therapeutic potential of a glucagon receptor antagonist (GRA) in post-myocardial infarction heart failure, the in vivo activity of a glucagon receptor antagonist is evaluated using myocardial infarction induced C57BL/6 mice. Myocardial infarction (MI) is induced in 56 C57BL6 male mice at the age of 8-10 weeks old by occluding the left coronary artery. After MI, mice are randomly divided into three groups. Group 1 is treated with the vehicle PBS as a control (CON, n=20); Group 2 is treated with 30 mg/kg MK-0893 at 2 hours and at 14 days post-MI (GRA, n=18); and Group 3 is treated with glucagon (30 μg/kg body weight in 10% gelatin), four times per day for the first six days post MI (GLC, n=18).

Infarcted mice are checked for survival every day for 28 days. Survival curves are delineated over time. Fasting blood glucose measurements are taken at 2 weeks post-MI. Cardiac function is monitored during the post-MI recovery. At the end of the 28 day study, the mice are euthanized and their hearts are weighed, photographed, and harvested for histological studies and ventricular contraction (echo FS/EF), ventricular pressure and contractility (catheter); ventricular remodeling, septum and LV wall thickness (echo), cardiac index (HW/BW), myocyte hypertrophy, fibrosis; infarct size, and apoptosis are determined.

Cardiac function is measured by echocardiography three times, the first day after MI, then at 14 and 28 days post-MI. The first echo is used to screen infarcted mice for no infarction. The mice with left ventricular fractional shortening greater than 40% are eliminated from the study. At the end of the experiment, ventricular function is measured by Micro-tip pressure transducer (catheter). For the histological studies, the heart is perfused with 10% formalin for fixation. Cardiac sections from the apex, middle, and base of the heart are used for Masson's trichrome staining (MTS) and TUNEL staining. Infarct size is measured in MTS sections, and apoptosis is assessed in the sections after TUNEL staining. The protective effect of MK-0893 is assessed based on infarct size and apoptosis. Fibrosis and myocyte size are measured in MTS sections for assessment of cardiac remodeling.

### EXAMPLE 2

The present inventors wish to evaluate the therapeutic potential of a glucagon receptor antagonist (GRA) on preventing heart failure/diabetic nephropathy induced by diabetes mellitus, using db/db and db/+ mice.

In this 12 week study, db/db and db/+mice (male, 8 weeks old) will be randomly divided into four groups. In Group 1, db/+mice are treated once a week with the vehicle PBS as a control (CON, n=8); in Group 2, db/+mice are treated once a week with 30 mg/kg MK-0893 (GRA, n=8); in Group 3, db/db mice are treated once a week with the vehicle PBS as a control (CON, n=15); and in Group 4, db/db mice are treated once a week with30 mg/kg MK-0893 (GRA, n=15). At the end of the 12 week study, the mice are euthanized and the hearts are harvested for histological studies.

Fasting blood glucose measurements are taken at 0, 4, 8, and 12 weeks after treatment. Cardiac function is measured by echocardiography at 0, 6 and 12 weeks after treatment. Cardiac function changes (e.g., ventricular systolic (echo/FS/EF) and diastolic (echo E/A and IRT) function, ventricular pressure and contractility (catheter), and cardiac output (echo)) are monitored at 0, 6 and 12 weeks after treatment. LV remodeling (e.g., septum and LV wall thickness (echo) is measured by Micro-tip pressure transducer (catheter) at 12 weeks after treatment. Cardiac index (HW/BW) is measured at 12 weeks after treatment. Kidney weight/tibial length, body weight, urinal albumin/creatinine, and GFR measurements are taken at day 0 and at 12 weeks after treatment.

For the histological studies, the heart is perfused with 10% formalin for fixation. Cardiac sections from the apex, middle, and base of the heart are used for Masson's trichrome staining (MTS) and TUNEL staining. Infarct size is measured in MTS sections, and apoptosis is assessed in the sections after TUNEL staining. Fibrosis and myocyte size were measured in MTS sections for assessment of cardiac remodeling.

### EXAMPLE 3

In this example, the in vivo activity of a glucagon receptor antagonist (GRA) is evaluated for efficacy in a miniature swine myocardial infarct model (Sinclair Research).

Miniature swine (young adult/female) are given a 7 day acclimation period and randomly divided into two groups. In Group 1, the swine are treated with a single dose of vehicle PBS as a control (CON, n=5) and in Group 2, the swine are treated with a single dose of 30 mg/kg MK-0893 (GRA, n=5). Prior to dose administration, each animal undergoes surgery (ligation of left ascending artery for 10-20 minutes) to create an occlusion and induce myocardial infarction. After 20 minutes, the occlusion is removed and the treatment administered during the reperfusion. The animals are maintained and handled in a stress-free environment post-op.

Mortality/Moribundity observations are made twice daily. Various assessments and parameters are made at baseline and prior to termination or necropsy. These include body weight, clinical observations, clinical pathology, clinical chemistry, hematology and coagulation parameters, and ECG/cardiac assessments. At the end of the study (28 days), limited necropsy is performed and limited heart tissue collected for histopathology (the middle of the infarct, the injected area and one more at each extent of the infarcted area is evaluated) for histological studies and ventricular contraction (echo FS/EF), ventricular pressure and contractility (catheter); ventricular remodeling, septum and LV wall thickness (echo), cardiac index (HW/BW), myocyte hypertrophy, fibrosis; infarct size, and apoptosis were determined.

### Additional Materials and Methods

### Preparation of Infarcted Mice

MI is induced in mice as described previously (Patten RD et al., Am J Physiol., 1998; 274:H1812-1820. [PubMed: 9612394]). Briefly, the chest is opened via a left thoracotomy. The left coronary artery is identified visually using a stereo microscope, and a 7-0 suture is placed around the artery 1-2 mm below the left auricle. The electrocardiogram (ECG) is monitored continuously. Permanent occlusion of the left coronary artery results from its ligation with the suture. Myocardial ischemia is confirmed by pallor in heart color and ST-segment elevation. The chest is closed with 6-0 silk suture. Once spontaneous respiration resumed, the endotracheal tube is removed. The animals are monitored until fully conscious. After they are returned to their cages, standard chow and water are provided.

### Echocardiography

In vivo cardiac function is assessed by transthoracic echocardiography (Acuson P300, 18 MHz transducer; Siemens) in conscious mice, as described previously. The mouse heart is imaged in the two-dimensional mode in the parasternal short axis at a papillary level. From this view, LV chamber dimensions are measured. Fractional shortening (FS) is calculated from left ventricular (LV) end-diastolic diameter (EDD) and end-systolic diameter (ESD). M-mode EDD and ESD are averaged from three to five beats. Studies and analyses are performed by investigators blinded to treatments.

### Measurement of LV Pressure by Mikro-Tip Catheter

Mice are anesthetized with pentobarbital (40 mg/kg, ip). The chest is opened via a left thoracotomy. The infarcted heart is exposed. A mouse Mikro-tip catheter (SPR1000) is inserted into the left ventricle through non-infarcted ventricular wall. LV pressure is measured by PowerLab system (Adinstruments). Ventricular contractility is calculated through Chart 7 software (Adinstruments).

### Histological Studies

Hearts were fixed with 10% buffered formalin, embedded in paraffin, and sectioned at 6 μm, as described previously [1]. One middle longitudinal section per heart was stained with Masson's trichrome. Eight randomly selected fields (400×) from the non-infarct area in the left ventricle were examined for fibrosis and myocyte size under a microscope. Each group comprised 5-6 hearts, and minimal 40 fields were analyzed in each group by computerized planimetry (NIH Image J). To assess fibrosis, fibrotic blue area and whole myocardial area were measured. The fibrotic area was presented as a percentage of fibrotic area to the myocardial area. Myocyte size was measured in cross-sectioned muscle cells. Total 100-150 cells/heart were analyzed. Two methods were used to assess the size of the infarcted heart. Infarct area was calculated as a percentage of infarcted ventricular area to total ventricular area using the front and back sides of the heart photos. Infarct size was measured as a percentage of infarcted ventricular wall length to total ventricular wall length using cardiac sections. The observer was blinded to the origin of the cardiac sections.

### TUNEL Assay

TUNEL assay was performed with the In Situ Apoptosis Detection Kit. Briefly, hearts were fixed by perfusion with 10% formalin solution, embedded in paraffin, and sectioned at 6 μm. One middle longitudinal section per heart was taken for TUNEL staining. Proteinase K (20 μg/ml) was added to each slide. Endogenous peroxidases was inactivated by covering sections with 2% hydrogen peroxide. After fixation, sections were incubated with TdT buffer at 37° C. for 30 min. Reactions were terminated with 1×SSC. After being washed, slides were incubated with RTU streptavidin-HRP for 30 min. Positive signal was developed by adding DAB solution. After counterstained with RTU hematoxylin, slides were covered by mounting medium and analyzed under a microscope. Each group comprised 5-6 hearts. Eight fields (400×) from the infarct area per heart were analyzed for positive cells and total cells using computerized planimetry (NIH Image J). The degree of apoptosis was presented as a percentage of positive cells to total cells.

All of the articles and methods disclosed and claimed herein can be made and executed without undue experimentation in light of the present invention. While the articles and methods of this invention have been described in terms of preferred embodiments, it will be apparent to those of skill in the art that variations may be applied to the articles and methods without departing from the spirit and scope of the invention. All such variations and equivalents apparent to those skilled in the art, whether now existing or later developed, are deemed to be within the spirit and scope of the invention as defined by the appended claims. All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes. The invention illustratively described herein suitably may be practiced in the absence of any element(s) not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for treating heart failure in a subject in need thereof comprising administering to a subject diagnosed with heart failure a therapeutically effective amount of a glucagon receptor antagonist.

2. A method according to claim 1, wherein the glucagon receptor antagonist is selected from LY2409021, MK-0893, GRA1, LGD-6972, PF-06291874 and Bat 27-9955.

3. A method according to claim 1, wherein the glucagon receptor antagonist is selected from [N-[(4-{(1S)-1-[3-(3,5-dichlorophenyl)-5-(6-methoxynaphthalen-2-yl)-1H-pyrazol-1-yl]ethyl}phenyl)carbonyl]-β-alanine]; [N-(4-{(1S)-1-[(R)-(4-chlorophenyl)(7-fluoro-5-methyl-1H-indo1-3-yl)methyl]butyl}benzoyl)-β-alanine];

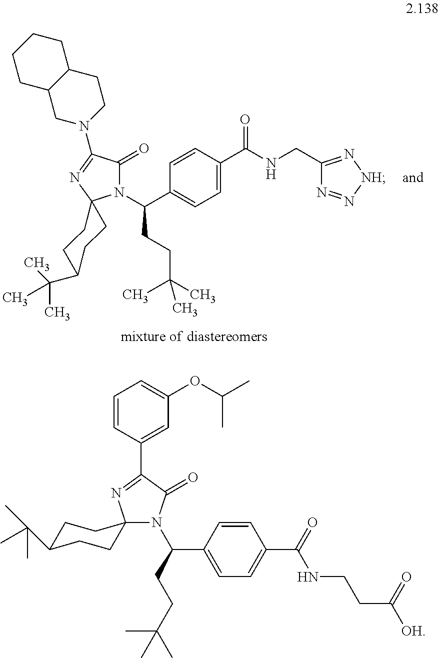 [SMILES: CC(C)(C)CC[C@H](c1ccc(C(=O)NCc2nn[nH]n2)cc1)N1C(=O)C(N2CCC3CCCCC3C2)=NC12CCC(C(C)(C)C)CC2 ; CC(C)Oc1cccc(C2=NC3(CCC(C(C)(C)C)CC3)N([C@H](CCC(C)(C)C)c3ccc(C(=O)NCCC(=O)O)cc3)C2=O)c1]

4. A method according to claim 1, wherein the subject is diagnosed with post-myocardial infarction heart failure.

5. A method according to claim 1, wherein the subject is diagnosed with diabetic cardiomyopathy heart failure.

6. A method of preventing heart failure in a subject who is at risk of developing heart failure comprising: (a) identifying a subject who is at risk of developing heart failure and (b) administering to the subject a therapeutically effective amount of a glucagon receptor antagonist.

7. A method according to claim 6, wherein the subject who is at risk of developing heart failure is identified as having experienced myocardial infarction.

8. A method according to claim 6, wherein the subject who is at risk of developing heart failure is identified as having diabetes mellitus or diabetic cardiomyopathy.

9. A method for treating heart failure in a subject in need thereof, comprising administering to the subject a therapeutically effective amount of an a glucagon receptor antagonist in combination with one or more anti-heart failure drugs selected from a group consisting of: angiotensin-converting enzyme (ACE) inhibitors, β-adrenergic blocking agents, angiotension II receptor blockers (ARBs), diuretics, and digitalis.
